SEQUENCE LISTING (25 sequences)

SEQ ID NO: 1 - protein, 311 aa, Homo sapiens
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 2 - protein, 191 aa, Homo sapiens
MHDSNNVEKDITPSELPANPGCLHSKEHSIKATLIWRLFFLIMFLTIIVCGMVAALSAIRANCHQEPSVCLQAACPESWIGFQRKCFYFSDDTKNWTSSQRFCDSQDADLAQVESFQELNFLLRYKGPSDHWIGLSREQGQPWKWINGTEWTRQFPILGAGECAYLNDKGASSARHYTERKWICSKSDIHV

SEQ ID NO: 3 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 4 - protein, 281 aa, Homo sapiens [sequence omitted]

SEQ ID NO: 5 - protein, 423 aa, Homo sapiens
MERMLPLLALGLLAAGFCPAVLCHPNSPLDEENLTQENQDRGTHVDLGLASANVDFAFSLYKQLVLKAPDKNVIFSPLSISTALAFLSLGAHNTTLTEILKGLKFNLTETSEAEIHQSFQHLLRTLNQSSDELQLSMGNAMFVKEQLSLLDRFTEDAKRLYGSEAFATDFQDSAAAKKLINDYVKNGTRGKITDLIKDLDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKAVLDVFEEGTEASAATAVKITLLSALVETRTIVRFNRPFLMIIVPTDTQNIFFMSKVTNPKQA

SEQ ID NO: 6 - protein, 376 aa, Homo sapiens
METLSNASGTFAIRLLKILCQDNPSHNVFCSPVSISSALAMVLLGAKGNTATQMAQALSLNTEEDIHRAFQSLLTEVNKAGTQYLLRTANRLFGEKTCQFLSTFKESCLQFYHAELKELSFIRAAEESRKHINTWVSKKTEGKIEELLPGSSIDAETRLVLVNAIYFKGKWNEPFDETYTREMPFKINQEEQRPVQMMYQEATFKLAHVGEVRAQLLELPYARKELSLLVLLPDDGVELSTVEKSLTFEKLTAWTKPDCMKSTEVEVLLPKFKLQEDYDMESVLRHLGIVDAFQQGKADLSAMSAERDLCLSKFVHKSFVEVNEEGTEAAAASSCFVVAECCMESGPRFCADHPFLFFIRHNRANSILFCGRFSSP

SEQ ID NO: 7 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 8 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 9 - protein, 506 aa, synthetic construct
MSRSVALAVLALLSLSGLEAGAVDPLLALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSMGSHSMRYFYTAVSRPGRGEPHFIAVGYVDDTQFVRFDSDAASPRGEPRAPWVEQEGPEYWDRETQKYKRQAQTDRVSLRNLRGYYNQSEAGSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQKAELVETRPAGDGTFQKWAAVVVPSGEEQRYTCHVQHEGLPEPLTLRWEPSSQPTIPIVGIVAGLAVLAVLAVLGAVVAVVMCRRKSSGGKGGSCSQAASSNSAQGSDESLIACKA

SEQ ID NO: 10 - protein, 506 aa, synthetic construct
MSRSVALAVLALLSLSGLEAQYDDAVYKLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSMGSHSMRYFSTSVSWPGRGEPRFIAVGYVDDTQFVRFDSDAASPRGEPREPWVEQEGPEYWDRETQKYKRQAQADRVNLRKLRGYYNQSEDGSHTLQRMFGCDLGPDGRLLRGYNQFAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQKAELVETRPAGDGTFQKWAAVVVPSGEEQRYTCHVQHEGLPEPLTLRWKPSSQPTIPIVGIVAGLAVLAVLAVLGAMVAVVMCRRKSSGGKGGSCSQAASSNSAQGSDESLIACKA

SEQ ID NO: 11 - protein, 506 aa, synthetic construct
MSRSVALAVLALLSLSGLEAIIDKSGIPVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSMCSHSMRYFYTAVSRPGRGEPRFIAVGYVDDTQFVQFDSDAASPRGEPRAPWVEQEGPEYWDRETQKYKRQAQTDRVNLRKLRGYYNQSEAGSHTLQRMYGCDLGPDGRLLRGYNQFAYDGKDYIALNEDLRSWTAADKAAQITQRKWEAAREAEQRRAYLEGTCVEWLRRYLENGKKTLQRAEHPKTHVTHHPVSDHEATLRCWALGFYPAEITLTWQRDGEDQTQKAELVETRPAGDGTFQKWAAVVVPSGEEQRYTCHVQHEGLPEPLTLRWGPSSQPTIPIVGIVAGLAVLAVLAVLGAVMAVVMCRRKSSGGKGGSCSQAASSNSAQGSDESLIACKA

SEQ ID NO: 12 - DNA, 933 bp, synthetic construct
  source 1..933: mol_type = other DNA
atgggggttcccactgcgctcgaagcgggctcctggagatggggttcattgcttttcgctttgtttttggcggcaagcctcggtccagttgcggccttcaaggttgcaactccatactccttgtacgtctgtcccgaaggtcagaatgtcaccctcacgtgtcgactgctggggcctgtcgataaaggtcacgacgtgacgttttacaagacctggtatcgatcatcaagaggggaagtacagacctgttccgagcggcggcctattaggaaccttacatttcaagacctgcacttgcaccacggcggacatcaggccgcaaatacatcccacgatctggcacagcggcatgggcttgaatctgctagcgatcaccacgggaatttctctattacgatgcggaatctgactctcctggactcagggctgtactgctgtttggttgtagagattcgccaccatcattcagagcatagggtacacggtgcgatggaactgcaagttcaaaccggtaaggacgctcccagcaactgtgtcgtttatccgagctcaagccaagatagcgagaacataaccgccgctgcccttgcaacaggggcctgtatcgtagggattctgtgcctgccattgatcctcctccttgtgtacaaacagcgccaggcagcgtctaatagacgcgcccaagagctcgtcagaatggacagtaatatccagggcatagagaatccagggtttgaagcatcccctccggcgcaggggattccagaagcaaaagttcgccatcctcttagctacgtggcccagcgacaaccctctgaaagtgggagacacctgctgtctgagccctcaacacctctgtctccgccaggtccgggagatgtttttttcccatccctcgatcctgtccctgattcacctaactttgaagttata

SEQ ID NO: 13 - DNA, 573 bp, synthetic construct
  source 1..573: mol_type = other DNA
atgcacgattcaaacaacgtggaaaaagacattaccccatccgaacttccagcaaatcctggttgccttcatagcaaggagcactctatcaaagcaacactgatttggcgcttgttcttccttataatgtttctcacaattatcgtttgcggtatggtagccgccttgagcgccattcgggccaattgccaccaggaaccatccgtatgcctgcaagctgcttgtcccgagagctggattgggtttcaacggaagtgcttttacttcagtgacgataccaaaaactggacatcatctcaacgcttctgcgatagtcaagacgctgatctggctcaggtagagagtttccaggaactgaactttctcctcagatataagggtccatctgatcattggattggactttctcgggaacaggggcaaccttggaaatggatcaatggtacggaatggacacgccagtttcctatactcggtgcaggtgaatgtgcgtatcttaatgacaagggagcgtcttctgcaaggcactatacagaacggaaatggatatgtagcaagagtgacatacacgta

SEQ ID NO: 14 - DNA, 843 bp, synthetic construct
  source 1..843: mol_type = other DNA
atgcaacaacccttcaattatccgtacccccagatttactgggttgattcttccgctagcagtccatgggctccgccgggaacagttttgccgtgcccgacttctgtacccagacgacctggacagcgaagaccaccccccccgcctccaccacctccactgccaccaccgcctcctcctccccctctcccaccccttccattgcccccgttgaagaagcgaggtaatcattcaactgggttgtgcctgctggtaatgttttttatggttcttgtagcactcgtcggccttggccttgggatgttccaattgtttcaccttcaaaaggaacttgccgaacttcgcgaatccacaagtcaaatgcacactgctagcagtttggagaaacaaattgggcatccgtcccctcctcctgagaaaaaagaactcagaaaagttgcgcatttgactgggaaatccaactctaggagtatgccacttgaatgggaggatacatacggaatagttctcctcagtggcgtgaagtacaaaaaggggggacttgtaattaacgaaaccggattgtattttgtctatagtaaggtgtattttcgaggtcaaagctgcaacaacctgccgctcagccacaaggtatatatgagaaatagtaagtacccacaggatcttgtcatgatggagggtaaaatgatgtcatattgcacgacaggtcagatgtgggcccgctcctcttacctgggtgcggtatttaacttgacttctgcagatcacctttatgtgaatgtatcagagctttctctggtgaactttgaggagagtcagacatttttcggactttataagttg

SEQ ID NO: 15 - DNA, 843 bp, synthetic construct
  source 1..843: mol_type = other DNA
atggcgatgatggaagtacaaggaggtcctagcctgggacagacgtgtgtcctgatagttattttcaccgttcttctccaatcactgtgtgtagcagtgacgtatgtatattttacaaatgaacttaaacaaatgcaggataagtactccaagtcaggaatcgcttgctttctcaaagaagacgacagctactgggacccgaacgacgaggaatcaatgaattcaccatgctggcaagtcaaatggcagcttcggcagctggtgaggaaaatgattcttcgaactagtgaagaaacaatttctaccgtacaggaaaaacaacaaaacatatctccacttgtccgcgaacgaggacctcagagggtagccgcacatatcacgggtacacgcggtcgctcaaataccttgtccagtcccaattccaaaaatgaaaaggcattgggaaggaaaatcaactcctgggagtctagcaggtctggtcatagcttcttgagtaatcttcatctccgaaatggtgagctcgtgattcacgaaaagggcttctactatatatattctcagacttatttccgctttcaggaggagatcaaagaaaatacaaaaaatgataagcagatggtgcaatatatttacaaatacacatcttacccagaccccattttgctgatgaaatccgcacgcaactcctgctggtctaaggatgctgagtacggattgtattccatttatcagggaggcatatttgaattgaaagagaatgatcgaatatttgtaagcgtcacaaacgagcatttgatagatatggaccatgaagcctcatttttcggtgctttcttggtgggg

SEQ ID NO: 16 - DNA, 1269 bp, synthetic construct
  source 1..1269: mol_type = other DNA
atggaacgaatgcttcccctgcttgctctggggttgctggccgctgggttttgcccggcggtattgtgccatccaaatagcccattggacgaagaaaatttgacgcaagagaatcaggatagaggcactcatgtggaccttggcttggcctccgctaacgtcgatttcgcattttctctctataaacagctggtccttaaagctcccgataaaaacgtaatatttagtccgctcagtatttcaacggcgctcgccttcttgtctcttggggcgcacaacaccacgttgacggaaattctgaaaggacttaaatttaacctcaccgaaacgtctgaggccgagattcaccaatcattccaacacctgctccgcacgttgaatcaaagctccgatgagcttcaactttctatgggtaatgcgatgttcgtgaaggagcaacttagcttgcttgataggtttacagaagatgcgaagaggctttacggaagtgaggcgttcgctacagacttccaagattcagccgcggcgaagaagctcatcaatgattacgtcaagaatgggactagaggaaaaataactgacttgattaaagatctggattcccaaacgatgatggtattggtcaattatattttctttaaggctaagtgggaaatgccttttgatcctcaagatacccaccaatctagattctacctctctaaaaagaaatgggtcatggtacctatgatgagtctgcaccatttgacaattccgtattttcgggacgaggagctgtcatgtacggtagtcgagctgaaatacaccggcaatgcttccgccctcttcatacttcccgaccaggataaaatggaagaagtcgaagccatgcttcttccggaaacccttaaacggtggcgagactcactcgaatttagagaaataggcgagctctaccttccaaagtttagcatttcacgagattacaacttgaacgatattctcctgcaattgggaatagaggaggctttcacatctaaagcggatttgagtggcattacgggcgctagaaatctggcagtgtcccaggttgttcacaaggcagtgttggatgtttttgaggaaggaaccgaagcaagtgccgcgacggcagtcaaaataacgctcctgagcgcactcgttgaaacgagaacgatagtgcggtttaatagaccgttcctgatgatcatagtcccaaccgacactcagaacattttctttatgtccaaggtgactaaccctaagcaagcg

SEQ ID NO: 17 - DNA, 1128 bp, synthetic construct
  source 1..1128: mol_type = other DNA
atggaaacactgtccaatgcgtccggtacctttgctatcaggctcctcaagattctctgtcaggataatccttcccacaacgttttctgcagccctgtgagtataagcagtgcgcttgcaatggttctgctgggcgctaaaggcaataccgcaacccaaatggcacaagcccttagtcttaataccgaggaggacatccacagggcattccaatccttgttgaccgaagtcaataaggcggggacccagtacttgctgcgcacggccaatagactgtttggcgagaaaacttgtcaatttctgtcaacattcaaagagtcttgtctgcagttttaccacgccgagttgaaggaactttcctttatcagggcggccgaggagtctcgcaaacacattaatacttgggtatcaaaaaagaccgaaggtaaaatcgaggaacttcttccaggatcctctatagatgccgaaacacggctggtgcttgtgaacgctatatacttcaaaggaaagtggaacgagccatttgatgagacctatacccgcgaaatgcccttcaaaataaaccaggaagagcagcggcctgtccagatgatgtaccaagaggccacattcaagcttgcccacgtaggagaagtgcgggcgcaactgctggagctgccttacgccaggaaagagcttagtctgctcgtgctgcttcccgatgatggggtcgaactttccactgttgagaaatccctcacttttgaaaagctcactgcctggactaagccggattgcatgaagagtacggaagtcgaagtgttgctgcccaaatttaagcttcaagaagactacgatatggagtccgttctccgacatttgggaatcgtcgatgcgttccagcaggggaaagctgatttgagcgccatgtctgccgaaagggacctgtgcctctctaagttcgtccataagtcttttgttgaagtaaacgaagagggcaccgaagcggccgcggcatcaagttgtttcgtagtagccgagtgctgcatggagagcggaccgagattctgcgcagaccatccgtttctcttttttatcagacacaaccgggcgaactcaattctgttttgcggtaggttctcttcccca

SEQ ID NO: 18 - DNA, 669 bp, synthetic construct
  source 1..669: mol_type = other DNA
atggcttgtctgggtttccagagacataaggcccaactgaatttggccacgcgaacgtggccgtgcacgcttttgtttttcttgctttttatcccggtcttttgcaaagcaatgcacgtggcacaaccggctgtcgtactggcctcaagcagaggaattgcaagttttgtttgtgagtacgcctcccccggcaaggctactgaagtcagagttactgtacttcgccaggctgattctcaagtcaccgaggtatgcgccgccacttatatgatggggaatgaactgacatttcttgacgattccatttgtactgggacttcctcaggcaatcaggtaaacttgactatccagggcttgagagccatggatactggtctttatatctgtaaggtggaactcatgtacccgcccccttactatttgggaattggtaacggaacccagatctatgtgatagaccctgaaccttgtcccgattcagatttcctgctttggattttggcggctgtaagtagtggcttgttcttttatagtttcttgcttactgcggtatccctctcaaagatgctgaagaaacgctctcccttgacaactggcgtatatgtgaaaatgccccccactgagccggagtgtgaaaagcagttccagccttactttatcccgattaac

SEQ ID NO: 19 - DNA, 969 bp, synthetic construct
  source 1..969: mol_type = other DNA
atgtggcctctcgtggctgcgcttctgctcggtagtgcttgttgcggttccgcgcagctcctttttaataagaccaaaagcgtagagttcaccttttgtaatgatactgtcgttattccctgcttcgtaacgaacatggaagcccaaaatacaactgaggtatatgttaaatggaagttcaagggccgcgatatctacaccttcgatggtgctttgaacaagagcactgtaccaaccgatttcagcagtgctaagattgaggttagccagcttttgaagggagacgcctcactgaaaatggacaagtccgacgcagtttcccacacgggcaattatacttgtgaagtaaccgagctcactagagaaggagaaacgataatcgaactgaaatatcgcgtggtgagctggtttagtccaaatgagaatatactgatcgtaatctttccaatcttcgctatactgcttttttggggtcagtttggaattaagacgctcaagtatcggtcagggggcatggatgaaaagaccattgccctgctggttgcgggactggttattacggtaatcgtcattgtgggtgcgatcctttttgtgccgggcgaatatagtcttaaaaacgcgacgggtttgggcttgattgtaacatctaccggcatccttatcctgctgcactactacgtcttttctacagctattggactgaccagctttgtcatcgcaattctggtgattcaggtaattgcgtatatacttgcagtagtaggtctttctttgtgtatagcggcctgcattcctatgcacggaccgctcttgataagcggactgtccatactcgctctcgcacagctgcttggacttgtttatatgaagtttgtcgcttccaatcagaaaacgattcagccacccagaaaggccgtggaagaaccattgaacgccttcaaggagtctaaaggaatgatgaatgatgag

SEQ ID NO: 20 - DNA, 1518 bp, synthetic construct
  source 1..1518: mol_type = other DNA
atgtctcgctccgtggccttagctgtgctcgcgctactctctctttctggcctcgaggctggggcggtagacccgctgttggcgcttggtggtggcggatccggtggtggcggttctggtggtggcggctccatccagcgtacgccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgcgacatgggtggtggcggttctggtggtggcggtagtggcggcggaggaagcggtggtggcggttccatgggctcccactccatgaggtatttctacaccgctgtgtcccggcccggccgcggggagccccacttcatcgcagtgggctacgtggacgacacgcagttcgtgcggttcgacagcgacgccgcgagtccgagaggggagccgcgggcgccgtgggtggagcaggaggggccggagtattgggaccgggagacacagaagtacaagcgccaggcacagactgaccgagtgagcctgcggaacctgcgcggctactacaaccagagcgaggccgggtctcacatcatccagaggatgtatggctgcgacgtggggcccgacgggcgcctcctccgcgggtatgaccagtacgcctacgacggcaaggattacatcgccctgaacgaggatctgcgctcctggaccgccgcggacacggcggctcagatcacccagcgcaagtgggaggcggcccgtgaggcggagcagctgagagcctacctggagggcctgtgcgtggagtggctccgcagatacctgaagaatgggaaggagacgctgcagcgcgcggaacacccaaagacacacgtgacccaccatcccgtctctgaccatgaggccaccctgaggtgctgggccctgggcttctaccctgcggagatcacactgacctggcagtgggatggggaggaccaaactcagaaagcggagcttgtggagaccaggccagcaggagatggaaccttccagaagtgggcagctgtggtggtgccttctggagaagagcagagatacacgtgccatgtgcagcacgaggggctgccggagcccctcaccctgagatgggagccgtcttcccagcccaccatccccatcgtgggcatcgttgctggcctggctgtcctggctgtcctagctgtcctaggagctgtggtggctgttgtgatgtgtaggaggaagagctcaggtggaaaaggagggagctgctctcaggctgcgtccagcaacagtgcccagggctctgatgagtctctcatcgcttgtaaagcc

SEQ ID NO: 21 - DNA, 1518 bp, synthetic construct
  source 1..1518: mol_type = other DNA
atgtctcgctccgtggccttagctgtgctcgcgctactctctctttctggcctcgaggctcaatacgatgatgcggtgtacaagttgggtggtggcggatccggtggtggcggttctggtggtggcggctccatccagcgtacgccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgcgacatgggtggtggcggttctggtggtggcggtagtggcggcggaggaagcggtggtggcggttccatgggctcccactccatgaggtatttctccacatccgtgtcctggcccggccgcggggagccccgcttcatcgcagtgggctacgtggacgacacgcagttcgtgcggttcgacagcgacgccgcgagtccaagaggggagccgcgggagccgtgggtggagcaggaggggccggagtattgggaccgggagacacagaagtacaagcgccaggcacaggctgaccgagtgaacctgcggaaactgcgcggctactacaaccagagcgaggacgggtctcacaccctccagaggatgtttggctgcgacctggggccggacgggcgcctcctccgcgggtataaccagttcgcctacgacggcaaggattacatcgccctgaacgaggatctgcgctcctggaccgccgcggacacggcggctcagatcacccagcgcaagtgggaggcggcccgtgaggcggagcagcggagagcctacctggagggcacgtgcgtggagtggctccgcagatacctggagaacgggaaggagacgctgcagcgcgcggaacacccaaagacacacgtgacccaccatcccgtctctgaccatgaggccaccctgaggtgctgggccctgggcttctaccctgcggagatcacactgacctggcagtgggatggggaggaccaaactcagaaagcggagcttgtggagaccaggccagcaggagatggaaccttccagaagtgggcagctgtggtggtgccttctggagaagagcagagatacacgtgccatgttcagcacgaggggctgccggagcccctcaccctgagatggaagccgtcttcccagcccaccatccccatcgtgggcatcgttgctggcctggctgtcctggctgtcctagctgtcctaggagctatggtggctgttgtgatgtgtaggaggaagagctcaggtggaaaaggagggagctgctctcaggctgcgtccagcaacagtgcccagggctctgatgagtctctcatcgcttgtaaagcc

SEQ ID NO: 22 - DNA, 1518 bp, synthetic construct
  source 1..1518: mol_type = other DNA
atgtctcgctccgtggccttagctgtgctcgcgctactctctctttctggcctcgaggctattatagacaagagcggcattccggtgggtggtggcggatccggtggtggcggttctggtggtggcggctccatccagcgtacgccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgcgacatgggtggtggcggttctggtggtggcggtagtggcggcggaggaagcggtggtggcggttccatgtgctcccactccatgaggtatttctacaccgccgtgtcccggcccggccgcggagagccccgcttcatcgcagtgggctacgtggacgacacgcagttcgtgcagttcgacagcgacgccgcgagtccaagaggggagccgcgggcgccgtgggtggagcaggaggggccggagtattgggaccgggagacacagaagtacaagcgccaggcacagactgaccgagtgaacctgcggaaactgcgcggctactacaaccagagcgaggccgggtctcacaccctccagaggatgtatggctgcgacctggggcccgacgggcgcctcctccgcgggtataaccagttcgcctacgacggcaaggattacatcgccctgaatgaggacctgcgctcctggaccgccgcggacaaggcggctcagatcacccagcgcaagtgggaggcggcccgtgaggcggagcagcggagagcctacctggagggcacgtgcgtggagtggctccgcagatacctggagaacgggaagaagacgctgcagcgcgcggaacacccaaagacacacgtgacccaccatcccgtctctgaccatgaggccaccctgaggtgctgggccctgggcttctaccctgcggagatcacactgacctggcagcgggatggcgaggaccaaactcagaaagcggagcttgtggagaccaggccagcaggagatggaaccttccagaagtgggcagctgtggtggtgccttctggagaagagcagagatacacgtgccatgtgcagcacgaggggctgccagagcccctcaccctgagatgggggccatcttcccagcccaccatccccatcgtgggcatcgttgctggcctggctgtcctggctgtcctagctgtcctaggagctgtgatggctgttgtgatgtgtaggaggaagagctcaggtggaaaaggagggagctgctctcaggctgcgtccagcaacagtgcccagggctctgatgagtctctcatcgcttgtaaagcc

SEQ ID NO: 23 - protein, 9 aa, synthetic construct
GAVDPLLAL

SEQ ID NO: 24 - protein, 9 aa, synthetic construct
QYDDAVYKL

SEQ ID NO: 25 - protein, 9 aa, synthetic construct
IIDKSGIPV

(19) United States
(12) Patent Application Publication
(10) Pub. No.: US 2025/0243462 A1
(43) Pub. Date: Jul. 31, 2025

(54) GENETICALLY MODIFIED CELLS FOR ENHANCED IMMUNE EVASION IN ALLOGENEIC CELLULAR THERAPIES

(71) Applicant: BlueRock Therapeutics LP, Cambridge, MA (US)

(72) Inventors: Sandra MILASTA, Cambridge, MA (US); Alejandro GOMEZ-ARISTIZABAL, Cambridge, MA (US); Wilhelmus Theodorus HENDRIKS, Cambridge, MA (US); Gregory MOTZ, Cambridge, MA (US)

(21) Appl. No.: 19/073,872

(22) Filed: Mar. 7, 2025

Related U.S. Application Data
(63) Continuation of application No. PCT/US2024/055753, filed on Nov. 13, 2024.
(60) Provisional application No. 63/598,521, filed on Nov. 13, 2023.
(60) Provisional application No. 63/572,755, filed on Apr. 1, 2024.
(60) Provisional application No. 63/679,512, filed on Aug. 5, 2024.

(51) Int. Cl. C12N 5/077; C07K 14/705; C07K 14/81; C12N 9/02

(52) U.S. Cl. CPC C12N 5/0657; C07K 14/7056; C07K 14/70578; C07K 14/8121; C12N 9/0008; C12N 2506/45; C12N 2510/00

(57) ABSTRACT

This disclosure provides methods and populations of cells, engineered to modulate the expression of select genes and thereby reduce natural killer cell mediated cytotoxicity. For example, this disclosure provides engineered cells equipped with one or more heterologous nucleic acid sequences encoding polypeptides that, when expressed, impede the typical cytotoxicity of natural killer cells as compared to comparable cell devoid of heterologous nucleic acid sequence.

### CROSS-REFERENCE TO RELATED APPLICATIONS

This application claims the benefit of priority of International Application No. PCT/US2024/055753, filed Nov. 13, 2024, which claims the benefit of priority of U.S. Provisional Application No. 63/598,521, filed Nov. 13, 2023, U.S. Provisional Application No. 63/572,755, filed Apr. 1, 2024, and U.S. Provisional Application No. 63/679,512, filed Aug. 5, 2024, the entire contents of which are each incorporated herein by reference.

### SEQUENCE LISTING

The instant application contains a Sequence Listing, which has been submitted via Patent Center. The Sequence Listing titled 627360SEQLIST.xml, which was created on Mar. 7, 2025, and is 38 kilobytes in size, is hereby incorporated by reference it its entirety.

### FIELD OF INVENTION

This disclosure relates generally to the field of cellular therapies. More particularly, this disclosure relates to genetically modified cells that exhibit enhanced immune evasion properties.

### BACKGROUND

Cellular therapies have gained prominence in recent years as a promising approach for treating certain diseases and conditions. One of the primary challenges facing successful therapeutic outcomes, especially with allogeneic cells, is the host immune response against transplanted cells. The immune response can lead to rejection of the transplanted cells, compromising the therapeutic efficacy and potentially leading to adverse reactions in the recipient.

Several strategies have been developed to address the issue of immune rejection. These include immunosuppressive drugs, donor-recipient matching, and co-stimulatory blockade, among others. However, these approaches come with limitations such as side effects, partial efficacy, and potential complications. Limitations in immune evasion persist and severely hinder the realization of genuine allogeneic therapies, compelling patients to rely on potentially harmful immunosuppressive drugs post-therapy and the risks of adverse reactions.

### SUMMARY

This disclosure provides methods and compositions of engineered cells, wherein the engineered cells include modifications that enable potent immune evasion capabilities. Supported by experimental results, this disclosure demonstrates the efficacy of modulating the expression of specific genes (e.g., CLEC2D, SERPINB9, and TRAIL) for diminishing or preventing natural killer cell immunotoxicity of engineered cells. In some instances, the engineered cells are engineered human pluripotent stem cells and the experimental results disclosed herein demonstrate these engineered cells can differentiate into specific cell types, including cardiomyocytes, while retaining reduced natural killer cell immunotoxicity and cell functionality. Consequently, this disclosure offers a promising avenue for reducing the reliance on external immunosuppression, ultimately allowing for safer and more potent allogeneic cell therapies.

In one aspect, this disclosure provides an engineered cell, the engineered cell comprising at least one heterologous nucleic acid sequence encoding a polypeptide, wherein the polypeptide is expressed at a level sufficient to inhibit immune cell mediated cytotoxicity (e.g., natural killer cell medicated cytotoxicity) of the engineered cell, and wherein the polypeptide comprises one or more of C-type lectin domain family 2 member D (CLEC2D), Tumor Necrosis Factor-related apoptosis-inducing ligand (TRAIL), serpin family B member 9 (SERPINB9), and human leukocyte antigen-C(HLA-C), or a variant thereof.

In some embodiments, the polypeptide comprises one or more of CLEC2D, TRAIL, and SERPINB9, or a variant thereof. In some embodiments, the polypeptide comprises CLEC2D or a variant thereof.

In some embodiments, the engineered cell comprises at least two different heterologous nucleic acid sequences, each encoding a distinct polypeptide. In some embodiments, the at least two different heterologous nucleic acid sequences encode CLEC2D or a variant thereof; and SERPINB9 or a variant thereof. In some embodiments, the at least two different heterologous nucleic acid sequences encode CLEC2D or a variant thereof; and TRAIL or a variant thereof. In some embodiments, the engineered cell comprises at least three different heterologous nucleic acid sequences, each encoding a distinct polypeptide.

In some embodiments, the polypeptide comprises an amino acid sequence that has at least 90% sequence identity to any one of SEQ ID NOS: 2, 4, 5, and 11. In some embodiments, the at least one heterologous nucleic acid sequence comprises a nucleotide sequence that has at least 90% sequence identity to any one of SEQ ID NOS: 13, 15, 17, and 22.

In some embodiments, the at least one heterologous nucleic acid sequence is integrated into a sustained transgene expression locus (STEL) or a sustained transcriptionally active payload region (STAPLR). In some embodiments, the STEL comprises a locus within a human glyceraldehyde 3-phosphate dehydrogenase (GAPDH) gene. In some embodiments, expression of the polypeptide is driven by an endogenous gene promoter.

In some embodiments, the engineered cell comprises a kill switch.

In some embodiments, the engineered cell further comprises a genetic modification that results in reduced T cell mediated killing as compared to a wild-type cell. In some embodiments, the genetic modification comprises: (i) a deletion, disruption, or attenuation of a B2 microglobulin (B2M) gene; or (ii) a deletion, disruption, or attenuation of one or more of an HLA-A gene, an HLA-B gene, and an HLA-C gene. In some embodiments, the engineered cell further comprises a second genetic modification that comprises deletion, disruption, or attenuation of a class II, major histocompatibility complex, transactivator (CIITA) gene or a regulatory factor X5 (RFX5) gene.

In some embodiments, the engineered cell comprises a stem cell. In some embodiments, the engineered cell comprises a cardiac cell, a neural cell, a myeloid cell, a T cell, an endocrine cell, an epithelial cell, a muscle cell, or a retinal cell. In some embodiments, the engineered cell is a human cell.

In another aspect, this disclosure provides a population of engineered cells derived from an engineered cell as described herein. In some embodiments, the population of engineered cells at least about 10% less susceptible to natural killer cell mediated cytotoxicity as compared to a population of cells not engineered to have the at least one heterologous nucleic acid sequence. In some embodiments, the population of engineered cells is at least about 20% less susceptible to natural killer cell mediated cytotoxicity as compared to a population of cells not engineered to have the at least one heterologous nucleic acid sequence. In some embodiments, at least 95% of the engineered cells in the population of engineered cells are positive for cardiac troponin T (cTnT).

In some embodiments, the population of engineered cells are present in a composition formulated for administration to a subject.

In another aspect, this disclosure provides a method of treating a disease or condition of a subject, the method comprising administering the population of engineered cells as described herein. In some embodiments, the disease or condition comprises heart failure, Parkinson's disease, multiple sclerosis, irritable bowel syndrome, type 1 diabetes, rheumatoid arthritis, cancer, liver disease, or neural inflammation. In some embodiments, the subject is a human.

In another aspect, this disclosure provides a method for achieving immune evasion, the method comprising: genetically engineering a cell to increase the expression of at least one polypeptide, wherein the at least one polypeptide comprises one or more of C-type lectin domain family 2 member D (CLEC2D), Tumor Necrosis Factor-related apoptosis-inducing ligand (TRAIL), serpin family B member 9 (SERPINB9), and human leukocyte antigen-C(HLA-C), or a variant thereof; and whereby said cell that has been genetically engineered exhibits increased survival when contacted with natural killer cells as compared to a cell not genetically engineered to increase the expression of the at least one polypeptide.

In some embodiments, the polypeptide comprises one or more of CLEC2D, TRAIL, and SERPINB9, or a variant thereof. In some embodiments, the polypeptide comprises CLEC2D or a variant thereof.

In some embodiments, the genetically modifying comprises integrating a heterologous nucleic acid sequence encoding the polypeptide into a genomic locus of the cell. In some embodiments, the genomic locus is a sustained transgene expression locus (STEL) or a sustained transcriptionally active payload region (STAPLR). In some embodiments, the STEL comprises a locus within a human glyceraldehyde 3-phosphate dehydrogenase (GAPDH) gene.

In some embodiments, the method further comprises expanding said cell that has been genetically engineered to produce a population of engineered cells. In some embodiments, the method further comprises differentiating the population of engineered cells into a population of cardiac cells, neural cells, T cells, retinal cells, endocrine cells, epithelial cells, muscle cells, or myeloid cells. In some embodiments, the cell is a human cell.

In another aspect, this disclosure provides a pharmaceutical composition comprising the population of engineered cells as described herein, and a pharmaceutical acceptable carrier, carrier, or dilutant.

In another aspect, this disclosure provides a kit comprising a dosage form suitable for administration to a subject comprising the population of engineered cells as described herein, and instructional material for the use of said dosage form.

In another aspect, this disclosure provides a kit comprising a dosage form suitable for administration to a subject comprising the pharmaceutical composition as described herein, and instructional material for the use of said dosage form.

### BRIEF DESCRIPTION OF DRAWINGS

FIG. 1 shows a schematic of an exemplary method of engineering induced pluripotent stem cells (iPSCs) to express a polypeptide of interest. A heterologous nucleic acid encoding the polypeptide of interest is integrated into a sustained transgene expression locus (STEL; e.g., GAPDH) in the genome of the iPSCs. Expression of the polypeptide of interest is then driven by the endogenous gene promoter.

FIG. 2 shows exemplary results of natural killer (NK) cell cytotoxicity assay evaluation of iPSCs expressing VISTA, CLEC2D, FASL, TRAIL, SERPINB9, CTLA4, CD47, or HLA-C challenged with NK cells from three different healthy human donors (pNK1, pNK2 and pNK3). The x-axis shows different iPSC modifications, including wild-type (WT) cells with various gene knock ins. The y-axis represents mean NK-specific cytotoxicity as a percentage with data shown separately for each NK cell healthy donor (pNK1: black bars, pNK2: medium gray bars, pNK3: light gray bars). Statistical significance is indicated by branching lines with p-values (<0.0001, <0.0001, <0.0002, and <0.001).

FIG. 3 shows exemplary results of NK cell cytotoxicity assay evaluation of iPSCs expressing VISTA, CLEC2D, FASL, TRAIL, SERPINB9, CTLA4, CD47, or HLA-C. Within a single column of the graph, each circle represents the NK cell mediated cytotoxicity from one of three distinct healthy donor NK cell populations (pNK1: black circles, pNK2: light gray triangles, pNK3: dark gray circles). The x-axis shows different engineered iPSCs, including wild-type (WT) control cells, modified with various gene knock ins as identified along the axis. The y-axis represents NK-specific cytotoxicity as a percentage. Data points show mean±SEM.

FIGS. 4A and 4B show exemplary results of natural killer (NK) cell cytotoxicity assay evaluation of engineered cardiomyocytes expressing FasL, TRAIL, HLA-C*04:01, CLEC2D, CTLA4, SERPINB9, HLA-C*05:01, or VISTA, challenged with NK cells from 4 different healthy donors (pNK1, pNK2, pNK3, and pNK4). FIG. 4A is a bar plot of the exemplary results. Gene targets are identified along the x-axis. The percent NK cell specific cytotoxicity is identified along the y axis. Statistical significance is indicated by branching lines with p-values (<0.0001, <0.0001, <0.0002, and <0.001). FIG. 4B is a table of the results shown in FIG. 4A (NS: p>0.05; *: p<0.05; **: p<0.01; ***: p<0.001; ****: p<0.0001).

FIGS. 5A-5C show exemplary results from the in vivo NK cell cytotoxicity evaluation of engineered cardiomyocytes expressing CLEC2D or SERPINB9. FIG. 5A shows the total photon flux of control cells (luciferase-positive cardiomyocytes) and control cells with healthy Donor 1 NK cells or Donor 2 NK cells over a period of 11 days. FIG. 5B shows the total photon flux from luciferase-positive cardiomyocytes engineered to express CLEC2D over a period of 11 days. FIG. 5C shows of the total photon flux from luciferase-positive cardiomyocytes engineered to express SERPINB9 over a period of 11 days. Total proton flux is indicated along the y axis. Time (days) is indicated along x axis.

FIG. 6 shows exemplary results from in vitro NK cell cytotoxicity assays of iPSCs engineered with CLEC2D, SERPINB9, or TRAIL, individually or in combination (i.e., CLEC2D+SERPINB9, CLEC2D+TRAIL). The x-axis shows different PSC modifications, including wild-type (WT) and B2M knockout (KO) cells with various gene knock ins. The y-axis represents NK-specific cytotoxicity as a percentage. The horizontal dotted line indicates baseline cytotoxicity levels for B2M-KO iPSCs. Each bar represents the mean cytotoxicity for results from all four NK cells from different healthy donors, with the mean results from each donor NK cell represented as a black triangle.

FIG. 7 shows exemplary results from in vitro NK cell cytotoxicity assays with different NK cell to iPSC ratios. The x-axis shows different engineered iPSCs, including B2M KO iPSCs, and iPSCs engineered with a B2M KO in combination with various gene knock-ins. The engineered iPSCs are grouped along the x-axis according to their NK: PSC ratio, i.e., 2:1 and 1:1 respectively. The y-axis represents NK-specific cytotoxicity as a percentage. Each bar represents the mean cytotoxicity for results from all three NK cells from different healthy donors, with the mean results from each donor NK cell represented as a black triangle.

FIG. 8 shows exemplary results from in vitro NK cell cytotoxicity assays with different NK cell to engineered cardiomyocyte (CM) ratios. The x-axis shows different engineered CMs, including B2M KO CMs, and CMs engineered with a B2M KO in combination with various gene knock-ins. The engineered CMs are grouped along the x-axis according to their NK: CM ratio, i.e., 2:1 and 1:1 respectively. The y-axis represents NK-specific cytotoxicity as a percentage. Each bar represents the mean cytotoxicity for results from all four NK cells from different healthy donors, with the mean results from each donor NK cell represented as a triangle, diamond or circle/dot.

FIG. 9 shows exemplary results from in vitro NK cell cytotoxicity assays of cardiomyocytes engineered with TRAIL alone or in combination with CLEC2D. The x-axis shows different engineered cardiomyocytes, including baseline control cells, TRAIL-expressing cells, and cells expressing both CLEC2D and TRAIL. The y-axis represents NK-specific cytotoxicity as a percentage. The dotted line indicates baseline cytotoxicity levels. Each bar represents the mean cytotoxicity, with individual data points shown as black triangles. Results were obtained at a 1:1 NK cell to cardiomyocyte ratio and demonstrate that the combination of CLEC2D and TRAIL provides enhanced protection against NK cell-mediated cytotoxicity compared to TRAIL alone or baseline conditions.

### DETAILED DESCRIPTION

Aspects of the present disclosure relate to methods, populations of engineered cells, pharmaceutical compositions, and kits that include an engineered cell that has increased survival when challenged with immune cells, such as, natural killer cells. In particular, the methods, populations of engineered cells, pharmaceutical compositions, and kits include an engineered cell that include a heterologous nucleic acid encoding a polypeptide expressed at a level sufficient to inhibit natural killer cell mediated cytotoxicity.

The immune response has an important role for the identification and elimination of foreign agents, such as pathogens or foreign antigens. For example, the presence of foreign antigens causes immune cells to mount an immune response against the foreign antigen. Presentation of foreign antigens on Major Histocompatibility Complex (MHC) molecules target a cell for killing by natural killer cells and cytotoxic T cells, thus clearing the foreign antigen. While natural killer cell and T cell cytotoxicity functions normally to eliminate pathogen-infected cells, tumor cells, and other deleterious agents, natural killer cell, and T cells can also target therapeutic agents such as cell therapies for clearance, thus reducing the therapeutic benefit conceded by these therapies. Thus, designing therapies that can overcome natural killer cell and T cell cytotoxicity is key to maintain an optimal therapeutic benefit.

In the embodiments described herein, provided are engineered cells that have increased immune evasion. In some embodiments, said engineered cells express a polypeptide at a sufficient level to inhibit natural killer (NK) cell-mediated cytotoxicity. By inhibiting natural killer cell cytotoxicity, the engineered cells have increased survival after administration into a subject. In some aspects of the embodiments described herein, the engineered cells can also include additional modifications that reduce killing of the cells by T cells, thus further increasing their survival in a subject. The increased survival of the engineered cells described herein lead to increased half-life of the cells when administered to a subject. By increasing the half-life of the engineered cells in a subject, the engineered cells can confer higher therapeutic benefit in the treatment of a disease or disorder in the subject.

Accordingly, it is an insight of the present disclosure that modifying the expression levels of one or more specific target genes within a cell can modulate that cell's susceptibility to NK cell-mediated cytotoxicity. In particular, the present disclosure provides experimental data demonstrating that increased expression of specific target genes, including CLEC2D, TRAIL, SERPINB9, and HLA-C, or a variant thereof, can render cells partially or completely resistant to NK cell recognition and subsequent cytolytic activity. The present disclosure provides methods and compositions for engineering enhanced cell survival through genetic manipulation of target genes, wherein overexpression of at least one target gene, such as CLEC2D, in human pluripotent stem cells results in reduced NK cell-mediated killing of said stem cells. Furthermore, this disclosure provides experimental data demonstrating said stem cells are capable of differentiating into downstream cell types, such as cardiomyocytes, while retaining cell functionality.

In addition, the present disclosure provides cells that are engineered to increase the expression of a combination of at least two target genes. The at least two target genes may be selected from the group consisting of CLEC2D, TRAIL, and SERPINB9, or a variant thereof. In some embodiments, at least one of said target genes is CLEC2D or a variant thereof. Exemplary combinations include, but are not limited to, cells modified to increase expression of both CLEC2D or a variant thereof and SERPINB9 or a variant thereof, or cells modified to increase expression of both CLEC2D or a variant thereof and TRAIL or a variant thereof. In some embodiments, the combination includes at least three target genes that include CLEC2D, SERPINB9, and TRAIL or a variant thereof. The disclosure contemplates that such modifications may be implemented in various cell types, including human pluripotent stem cells and differentiated cell types derived therefrom, such as cardiomyocytes.

The present disclosure further provides experimental validation of reduced NK cell immuno-cytotoxicity through the introduction of heterologous nucleic acids encoding one or more target genes, including CLEC2D or a variant thereof, into target cells. In particular embodiments, increased expression of the at least two target genes is achieved through integration of heterologous nucleic acids encoding said target genes into the genome of the target cell. Such integration may be accomplished through various methodologies known in the art, including genome editing systems such as CRISPR-based approaches or, alternatively, through lentiviral transduction. While the exemplary embodiments described herein utilize heterologous gene introduction, one of ordinary skill in the art will readily appreciate that alternative methodologies for increasing expression of the target genes fall within the scope of the present disclosure. Such alternative approaches include, but are not limited to, manipulation of endogenous gene expression through modification of regulatory elements such as promoters, enhancers, or other control elements associated with the target genes disclosed herein. The disclosure thus contemplates that any methodology resulting in increased expression of the identified target genes, whether through heterologous or endogenous means, may be employed to achieve the NK cell-resistant phenotype described herein.

Although the disclosure describes various exemplary alternatives and implementations as provided herein, it should be understood that the various features, aspects, and functionality described in one or more of the individual alternatives are not limited in their applicability to the particular alternative with which they are described. Instead, they can be applied alone or in various combinations to one or more of the other alternatives of the disclosure, whether the alternatives are described or whether the features are presented as a part of the described alternative. The breadth and scope of the present disclosure should not be limited by any exemplary alternatives described or shown herein.

### I. Definitions

The following definitions supplement those in the art and are directed to the present disclosure only. The following definitions are not to be imputed to any related or unrelated case, e.g., to any commonly owned patent or patent application. Although some methods and materials similar or equivalent to those described herein can be used to practice features of the disclosure, some preferred materials and methods are described herein. Accordingly, the terminology used herein is for the purpose of describing particular embodiments only and is not intended to be limiting.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only and is not intended to limit the scope of the present invention, which is defined solely by the claims.

As used herein, the articles “a,” “an,” and “the” are used herein to refer to one or to more than one (i.e., to at least one) of the grammatical object of the article. By way of example, “an element” means one element or more than one element.

The use of the alternative (e.g., “or”) should be understood to mean either one, both, or any combination thereof of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% compared to a reference quantity, level, value, number, frequency, percentage, dimension, size, amount, weight or length. In some instances, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or ±1% of a reference quantity, level, value, number, frequency, percentage, dimension, size, amount, weight or length.

As used herein, the terms “administration,” “administering” and variants thereof refer to the introduction of a composition or therapeutic agent (e.g., a population of cells) into a subject. Administration includes concurrent and sequential introduction of the composition or therapeutic agent. Administration of the composition or therapeutic agent (e.g., a population of cells) into a subject is by any suitable route, including orally, pulmonarily, intranasally, parenterally (intravenously, intramuscularly, intraperitoneally, or subcutaneously), rectally, intralymphatically, or topically. A suitable route of administration allows the composition or the agent to perform its intended function. Administration also includes self-administration and the administration by another. The administration can also be performed systemic, or it can be local. For instance, a composition or therapeutic agent (e.g., a population of cells) can be administered locally, e.g., by local injection into a tissue.

As used herein, the term “and/or” should be understood to mean either one, or both of, or any combination of the alternatives.

As used herein, the terms “β₂ microglobulin” and “B2M” are used interchangeably to refer to a polypeptide encoded by the B2M gene. B2M is a component of class I Major Histocompatibility Complexes (MHCs) and forms a complex with MHC class I human leukocyte antigens (HLAs). B2M is also known as IMD43; MHC1D4; AMYLD6; beta-2-microglobulin; and B2 microglobulin. In some instances, the B2M is a human B2M. An exemplary B2M gene is available as Gene ID: 567 (e.g., available at the website www[dot]ncbi[dot]nlm[dot]nih[dot]gov/gene/567). An exemplary nucleotide sequence that can be used to generate B2M or modify the B2M gene is available as Transcript ID NM_004048.4 (e.g., available at the website www[dot]ncbi[dot]nlm[dot]nih[dot]gov/nuccore/NM_004048.4).

As used herein, the term “cardiac cell” refers to any cell present in the heart that provides a cardiac function, such as heart contraction or blood supply, or otherwise serves to maintain the structure of the heart. The cardiac cell may be a cell of the epicardium, myocardium or endocardium of the heart. Cardiac cells also include, but are not limited to, cardiac muscle cells or cardiomyocytes, and cells of the cardiac vasculatures, such as cells of a coronary artery or vein. Other non-limiting examples of cardiac cells include epithelial cells, endothelial cells, fibroblasts, cardiac stem or progenitor cells, cardiac conducting cells and cardiac pacemaking cells that constitute the cardiac muscle, blood vessels and cardiac cell supporting structure.

As used herein, the terms “C-type lectin domain family 2 member D” and “CLEC2D” are used interchangeably to refer to a polypeptide encoded by the CLEC2D gene. CLEC2D is also known as natural killer cell receptor C-type lectin; CLAX; LLT1; Osteoclast Inhibitory Lectin (OCIL); C-type lectin domain family 2 member D; and C-type lectin domain family 2 member D. In some instances, the CLEC2D is a human CLEC2D. An exemplary CLEC2D gene is available as Gene ID: 29121 (e.g., available at the website www[dot]ncbi[dot]nlm[dot]nih[dot]gov/gene/29121). Exemplary nucleotide sequences that can be used to generate CLEC2D are available as Transcript ID NM_001004419.5 (e.g., available at the website www[dot]ncbi[dot]nlm[dot]nih[dot]gov/nuccore/NM_001004419.5), Transcript ID NM_001197317.3 (e.g., available at the website https://www[dot]ncbi[dot]nlm[dot]nih[dot]gov/nuccore/NM_001197317 [dot] 3), Transcript ID NM_001197318.3 (e.g., available at the website https://www[dot]ncbi[dot]nlm[dot]nih[dot]gov/nuccore/NM_001197318 [dot] 3), Transcript ID NM_001197319.3 (e.g., available at the website https://www[dot]ncbi[dot]nlm[dot]nih[dot]gov/nuccore/NM_001197319 [dot] 3), Transcript ID NM_013269.6 (e.g., available at the website https://www[dot]ncbi[dot]nlm[dot]nih[dot]gov/nuccore/NM_013269 [dot] 6), Transcript ID ENST00000261340 (e.g., available at the website https://useast[dot]ensembl[dot] org/Homo_sapiens/Transcript/Sequence_cDNA?db=core;g=E NSG00000069493;r=12:9664969-9699553;t=ENST00000261340), Transcript ID ENST00000430909 (e.g., available at the website https://useast[dot]ensembl[dot] org/Homo_sapiens/Transcript/Sequence_cDNA?db=core;g=E NSG00000069493;r=12:9664969-9699553;t=ENST00000430909), Transcript ID ENST00000466035 (e.g., available at the website https://useast[dot]ensembl[dot] org/Homo_sapiens/Transcript/Sequence_cDNA?db=core;g=E NSG00000069493;r=12:9664969-9699553;t=ENST00000466035), Transcript ID ENST00000460309 (e.g., available at the website https://useast[dot]ensembl[dot] org/Homo_sapiens/Transcript/Sequence_cDNA?db-core;g-E NSG00000069493;r=12:9664969-9699553;t=ENST00000460309), Transcript ID ENST00000261339 (e.g., available at the website https://useast[dot]ensembl[dot] org/Homo_sapiens/Transcript/Sequence_cDNA?db-core;g-E NSG00000069493;r=12:9664969-9699553;t=ENST00000261339), Transcript ID ENST00000543300 (e.g., available at the website https://useast[dot]ensembl[dot] org/Homo_sapiens/Transcript/Sequence_cDNA?db-core;g=E NSG00000069493;r-12:9664969-9699553;t=ENST00000543300), Transcript ID ENST00000544322 (e.g., available at the website https://useast[dot]ensembl[dot] org/Homo_sapiens/Transcript/Sequence_cDNA?db-core;g=E NSG00000069493;r=12:9664969-9699553;t=ENST00000544322) and Transcript ID ENST00000545918 (e.g., available at the website https://useast[dot]ensembl[dot] org/Homo_sapiens/Transcript/Sequence_cDNA?db-core;g=E NSG00000069493;r=12:9664969-9699553;t=ENST00000545918); as well as the nucleotide sequence provided herein in as SEQ ID NO: 13. Exemplary amino acid sequences of CLEC2D are available as Protein ID NP_001004419.1 (e.g., available at the website www[dot]ncbi[dot]nlm[dot]nih[dot]gov/protein/NP_001004419.1), Protein ID NP_001184246.1 (e.g., available at the website https://www[dot]ncbi[dot]nlm[dot]nih[dot]gov/protein/NP_001184246 [dot] 1), Protein ID NP_001184247.1 (e.g., available at the website https://www[dot]ncbi[dot]nlm[dot]nih[dot]gov/protein/NP_001184247 [dot] 1), Protein ID NP_001184248.1 (e.g., available at the website https://www[dot]ncbi[dot]nlm[dot]nih[dot]gov/protein/NP_001184248 [dot] 1), Protein ID NP_037401.1 (e.g., available at the website https://www[dot]ncbi[dot]nlm[dot]nih[dot]gov/protein/NP_037401 [dot] 1), Protein ID ENST00000261340 (e.g., available at the website https://useast[dot] 15nsemble [dot] org/Homo_sapiens/Transcript/Sequence_Protein?db=core;g=ENSG00000069493;r=12:9664969-9699553;t=ENST00000261340), Protein ID ENST00000430909 (e.g., available at the website https://useast[dot] 15nsemble [dot] org/Homo_sapiens/Transcript/Sequence_Protein?db=core;g=ENSG00000069493;r-12:9664969-9699553;t=ENST00000430909), Protein ID ENST00000466035 (e.g., available at the website https://useast[dot] 15nsemble [dot] org/Homo_sapiens/Transcript/Sequence_Protein?db=core;g=ENSG00000069493;r=12:9664969-9699553;t=ENST00000466035), Protein ID ENST00000460309 (e.g., available at the website https://useast[dot] 15nsemble [dot] org/Homo_sapiens/Transcript/Sequence_Protein?db=core;g=ENSG00000069493;r-12:9664969-9699553;t=ENST00000460309), Protein ID ENST00000261339 (e.g., available at the website https://useast[dot] 15nsemble [dot] org/Homo_sapiens/Transcript/Sequence_Protein?db=core;g=ENSG00000069493;r=12:9664969-9699553;t=ENST00000261339), Protein ID ENST00000543300 (e.g., available at the website https://useast[dot] 15nsemble [dot] org/Homo_sapiens/Transcript/Sequence_Protein?db=core;g=ENSG00000069493;r-12:9664969-9699553;t=ENST00000543300), Protein ID ENST00000544322 (e.g., available at the website https://useast[dot] 15nsemble [dot] org/Homo_sapiens/Transcript/Sequence_Protein?db=core;g=ENSG00000069493;r=12:9664969-9699553;t-ENST00000544322) and Protein ID ENST00000545918 (e.g., available at the website https://useast[dot]ensembl[dot] org/Homo_sapiens/Transcript/Sequence_Protein?db-core;g=E NSG00000069493;r=12:9664969-9699553;t=ENST00000545918); as well as the amino acid sequence provided herein in as SEQ ID NO: 2

As used herein, the terms “class II, major histocompatibility complex, transactivator” and “CIITA” are used interchangeably to refer to a polypeptide encoded by the CIITA gene. CIITA is also known as C2TA; CIITAIV; MHC2TA; NLR family, acid domain containing; NLRA; and nucleotide-binding oligomerization domain, leucine rich repeat and acid domain containing. In some instances, the CIITA is a human CIITA. An exemplary CIITA gene is available as Gene ID: 4261 (e.g., available at the website www[dot]ncbi[dot]nlm[dot]nih[dot]gov/gene/4261). An exemplary nucleotide sequence that can be used to generate CIITA or modify the CIITA gene is available as Transcript ID NM_000246.4 (e.g., available at the website www[dot]ncbi[dot]nlm[dot]nih[dot]gov/nuccore/NM_000246.4).

As used herein, the terms “cluster of differentiation 47” and “CD47” are used interchangeably to refer to a polypeptide encoded by the (I) 47 gene. CD47 is also known as integrin associated protein; IAP; MER6; OA3; CD47 molecule; CD47 antigen; Rh-related antigen; integrin-associated signal transducer; antigen identified by monoclonal antibody 1D8; antigenic surface determinant protein OA3; leukocyte surface antigen CD47; and CD47 glycoprotein. In some instances, the CD47 is a human CD47. An exemplary CD47 gene is available as Gene ID: 961 (e.g., available at the website www[dot]ncbi[dot]nlm[dot]nih[dot]gov/gene/961). An exemplary nucleotide sequence that can be used to generate CD47 is available as Transcript ID NM_001382306.1 (e.g., available at the website www[dot]ncbi[dot]nlm[dot]nih[dot]gov/nuccore/NM_001382306.1), as well as the nucleotide sequence provided herein in as SEQ ID NO: 19. An exemplary amino acid sequence of CD47 is available as Protein ID NP_001369235.1 (e.g., available at the website www[dot]ncbi[dot]nlm[dot]nih[dot]gov/protein/NP_001369235.1), as well as the amino acid sequence provided herein in as SEQ ID NO: 8.

As used herein, the terms “decrease” and “inhibit” are interchangeable and refer to any statistically significant reduction in biological activity (e.g., natural killer cell mediated toxicity) of a reference protein or cell. For example, an inhibition in biological activity can refer to a reduction of about 10%, 20%, 30%, 40%, 50%, 60%, 70%, 80%, 90%, or 100% in biological activity as compared to a control.

As used herein, the term “differentiation,” and its grammatical equivalents, refers to a process by which a stem cell or progenitor cell alters from one cell type to a more specialized cell type. Each specialized cell type in an organism can express a subset of all the genes that constitute the genome of the cell. Each cell type can be defined by its particular pattern of regulated gene expression. Cell differentiation can thus be described as a transition of a cell from one cell type to another cell type coincident with a switch from one pattern of gene expression to another.

As used herein, the term “dosage form” refers to a discrete amount of a composition comprising a predetermined amount of the active ingredient (e.g., a population of cells). The amount of the active ingredient is generally equal to the dosage of the active ingredient that would be administered to a subject or a convenient fraction of such a dosage such as, for example, one-half or one-third of such a dosage. The relative amounts of the active ingredient, the pharmaceutically acceptable excipient, and any additional ingredients in a pharmaceutical composition will vary, depending upon the identity, size, and condition of the subject treated and further depending upon the route by which the composition is to be administered. The dosage form can further include one or more additional pharmaceutically active agents. In some cases, the dosage form is for administration by injection into a subject.

As used herein, the term “encoding” refers to the property of specific sequences of nucleotides in a nucleic acid, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene or at least the exons of a gene encodes a protein if transcription and translation of mRNA corresponding to that gene produces the protein in a cell or other biological system. Both the coding strand, the nucleotide sequence of which is identical to the mRNA sequence, and the non-coding strand, used as the template for transcription of a gene or cDNA, can be referred to as encoding the protein or other product of that gene or cDNA.

As used herein, the term “endogenous” refers to a gene, nucleic acid, polypeptide, etc., that is normally present in a particular cell. For example, an endogenous gene may be a gene that is normally present in the genome of a cell.

As used herein, the term “expression” refers to the transcription and/or translation of a particular nucleotide sequence in a cell.

As used herein, the terms “enhance” and “increase” are interchangeable and refer to any statistically significant increase in biological activity (e.g., survival) of a reference protein or cell. For example, an increase in biological activity can refer to an increase of about 10%, 20%, 30%, 40%, 50%, 60%, 70%, 80%, 90%, or 100% in biological activity as compared to a control.

As used herein, the term “genetic modification” or “genetic alteration” refers to a change at the DNA level of a cell. A genetic modification includes an insertion, deletion, or substitution, typically within a defined sequence or genomic locus. In some instances, the genetic modification can be a deletion, disruption, or attenuation of a gene that results in a reduction or loss of expression of the encoded gene product. In other instances, the genetic modification includes the integration of a nucleotide sequence heterologous to the genomic locus. The genetic modification can be at a single nucleotide position or at multiple nucleotides, e.g., 2, 3, 4, 5 or more nucleotides, typically in close proximity to each other, e.g., contiguous nucleotides. Depending on the nature of the genetic modification, the expression of the gene can be up-regulated or down-regulated.

As used herein, the term “genetically modified” or “engineered” when used in the context of a cell refers to a cell that includes one or more genetic modifications, and which is not found in nature. The engineered cells can be made by any method known in the art, such as by manipulating the genome of the cell or inserting a new nucleic acid into the cell. For instance, a cell can be modified by integrating a nucleic acid encoding a gene of interest into a cell using a genome editing technique, such as a CRISPR/Cas9 system or a CRISPR/Cas12a system. In some instances, a cell can be modified by integrating a nucleic acid encoding a gene of interest into a cell with Lentiviral transduction.

As used herein, the term “heart failure” refers to a disease characterized by the inability of the heart to pump sufficient blood to fulfill the needs of the tissues and organs of the body. Heart failure can be caused by an abnormally low cardiac output.

As used herein, the term “heterologous,” when used in reference to a nucleic acid and/or polypeptide that is introduced to a host cell, refers a nucleic acid and/or polypeptide that is not naturally found in the host cell or naturally found at a given position in the genome of the host cells. For example, a construct is heterologous to a host cell if it contains some homologous sequences arranged in a manner not found in the host cell and/or the construct contains some heterologous sequences not found in the host cell.

As used herein, the terms “human leukocyte antigen” and “HLA” are used interchangeably to refer to a subunit of Major Histocompatibility Complexes (MHCs). MHCs are glycoproteins that presents antigens to immune cells, such as T cells. Class I MHCs are expressed in most nucleated cells and interact with CD8 receptors on the surface of T cells. Class I MHCs are composed of a class I HLA molecule and B2 microglobulin (B2M). Class II MHCs are usually present only in antigen presenting cells and interact with CD4 receptors on the surface of T cells. Class II MHCs are heterodimers of two Class II HLAs.

As used herein, the terms “human leukocyte antigen-A” and “HLA-A” are used interchangeably to refer to a polypeptide encoded by an HLA-A gene. HLA-A is a class I HLA. HLA-A is also known as HLAA.

As used herein, the terms “human leukocyte antigen-B” and “HLA-B” are used interchangeably to refer to a polypeptide encoded by an HLA-B gene. HLA-B is a class I HLA. HLA-B is also known as AS, HLAB; and B-4901. An exemplary HLA-B gene is available as Gene ID: 3106 (e.g., available at the website www[dot]ncbi[dot]nlm[dot]nih[dot]gov/gene/3106).

As used herein, the terms “human leukocyte antigen-C” and “HLA-C” are used interchangeably to refer to a polypeptide encoded by an HLA-C gene. HLA-C is a class I HLA. HLA-C is also known as D6S204; HLA-JY3; HLAC; HLC-C; and PSORS1. An exemplary HLA-C gene is available as Gene ID: 3107 (e.g., available at the website www[dot]ncbi[dot]nlm[dot]nih[dot]gov/gene/3107). An exemplary nucleic acid sequence that can be used to generated HLA-C is provided herein in as any one of SEQ ID NOS: 20-22. An exemplary amino acid sequence of HLA-C is provided herein in as any one of SEQ ID NOS: 9-11.

As used herein, the term “immune cell” refers to a cell of hematopoietic origin functionally involved in the initiation and/or execution of an immune response in an organism. An immune cell can be part of the innate and/or adaptive immune system. Exemplary immune cells include, but are not limited to, cells of the myeloid lineage (e.g., neutrophils, dendritic cells, eosinophils, mast cells, basophils, monocytes, microglia, and precursors thereof), as well as cells of the lymphoid lineage (e.g., T cells, B cells, natural killer cells, and precursors thereof).

As used herein, the term “instructional material” refers to a publication, a recording, a diagram, or any other medium of expression that can be used to communicate the usefulness of the compositions and methods of using the compositions associated with the publication, recording, diagram or other medium of expression. The instructional material of a kit of the disclosure can, for example, be affixed to a container that contains the population of cells and/or pharmaceutical composition of the disclosure or be shipped together with a container that contains the population of cells and/or pharmaceutical composition. Alternatively, the instructional material can be shipped separately from the container with the intention that the instructional material and the compositions be used cooperatively by the recipient.

As used herein, the term “kill switch” refers to a nucleic acid sequence that when expressed under certain conditions in a host cell causes the host cell to undergo apoptosis. Exemplary kill switch nucleic acid sequences include, but are not limited to, nucleotide sequence that encode a herpes simplex virus thymidine kinase (HSV-TK), an inducible caspase9 (incasep 9, iCasp9), CD20, and a mutant human thymidylate kinase (mTMPK). In some instances, the kill switch gene is inducible, wherein the kill switch is only activated upon the addition of an activator, e.g., a small molecule drug.

As used herein, the term “myeloid cell” refers to a cell of the myeloid lineage. Exemplary myeloid cell types include, but are not limited to, monocytes, microglia, macrophages, dendritic cells, basophils, eosinophils, erythrocytes, mast cells or neutrophils, and any precursor cells or any intermediate progenitor thereof.

As used herein, the term “natural killer cell” refers to a type of cytotoxic lymphocyte, part of the innate lymphoid cells, that expresses CD56, also called NCAM1, and/or any of the Natural cytotoxicity triggering receptors, and Killer Cell Lectin Like Receptor F1, also called NKp80; and lacks expression of CD3. NK cells are involved in clearance of cells infected with viruses and other intracellular pathogens, as well as in the removal of tumor cells and senescent cells. “Natural killed cell mediated cytotoxicity” refers to the killing of cells by NK cells.

As used herein, the term “neural cell” refers to any cells belonging to the nervous system. Exemplary neural cells include, without limitation, neurons and neuron precursor cells (irrespective of any specific neuronal subtype, e.g., including dopaminergic neurons, cortical neurons, spinal or oculomotor neurons, enteric neurons, interneurons, and trigeminal or sensory neurons), microglia and microglia precursor cells, glial cells and glial precursor cells (irrespective of any specific glial subtype, e.g., including oligodendrocytes, astrocytes, dedicated oligodendrocyte precursor cells and bipotent glial precursors, which may give rise to astrocytes and oligodendrocytes), Placode-derived cells, Schwann cells, and satellite cells.

As used herein, the term “neural inflammation” refers to inflammation of the nervous tissue of the central nervous system, such as the brain or the spinal cord. Neural inflammation can be caused by any means, including but not limited to infection, traumatic brain injury, toxic metabolites, ischemia reperfusion injury or autoimmunity. Neural inflammation can be acute, chronic, or both.

As used herein, the term “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like.

As used herein, the term “Parkinson's disease” refers to a neurodegenerative disorder characterized by extensive degeneration of dopaminergic neurons in the substantia nigra region in the brain. Parkinson's disease manifest in changes of both motor and non-motor functions. Exemplary changes in motor functions that are symptomatic of Parkinson's disease include, but are not limited to, tremor, hypokinesia, postural instability, abnormal gait and swallowing disturbances. Exemplary changes in non-motor functions that are symptomatic of Parkinson's disease include, but are not limited to, autonomic and neuropsychiatric disturbances such as anosmia or sleep abnormalities. Parkinson's disease can be characterized as early Parkinson's disease, or it can be characterized as advanced Parkinson's disease, with more severe symptoms in the more advanced stages of the disease.

As used herein, the terms “patient,” “subject,” “individual,” and the like are used interchangeably and refer to any animal, or cells thereof, whether in vitro or in situ, amenable to the compositions and methods described herein. In some instances, the patient, subject or individual is a human.

As used herein, the term “pharmaceutically acceptable excipient, carrier or diluent” refers to any material which, when combined with an active ingredient (e.g., a population of cells), allows the ingredient to retain biological activity and is non-reactive with the subject's immune system. Examples include, but are not limited to, any of the standard pharmaceutical excipients, carriers, or diluents, such as a phosphate buffered saline solution, normal saline, water, emulsions such as oil/water emulsion, and various types of wetting agents.

As used herein, the terms “polypeptide” and “protein” refer to a polymer of amino acid residues of any length. A polypeptide may include naturally-occurring modifications to one or more of the amino acids in the polymer. For instance, a polypeptide may include disulfide bond formation, glycosylation, lipidation, acetylation, or phosphorylation. A polypeptide may also include amino acid analogs or non-naturally occurring amino acids. Polypeptides may occur as a single chain or as associated chains.

As used herein, the term “promoter”, and its grammatical equivalents, refers to a region of a nucleic acid positioned upstream of a gene where relevant proteins (such as RNA polymerase and transcription factors) bind to initiate transcription of the gene. The promoter can be the promoter of an endogenous gene (e.g., GAPDH). The promoter can be a tissue-specific promoter. For example, a promoter of a gene that is turned on or off in certain cell or tissue types. The promoter can be an engineered promoter. For example, the promoter can be engineered to include one or more elements that can enhance or reduce expression of a gene. The one or more elements can include an enhancer. The promoter can be a synthetic promoter. For example, the promoter can comprise a non-naturally occurring sequence that is engineered to express a gene of interest in one or more cell types as a desired level of expression. The promoter can be a heterologous promoter.

As used herein, the terms “regulatory factor X5” and “RFX5” are used interchangeably to refer to a polypeptide encoded by the RFX5 gene. RFX5 is also known as MHC2D3; and MHC2D5. In some instances, the RFX5 is a human RFX5. An exemplary RFX5 gene is available as Gene ID: 5993 (e.g., available at the website https://www.ncbi.nlm.nih.gov/gene/5993). An exemplary nucleotide sequence that can be used to modify the RFX5 gene is available as Transcript ID NM_000449.4 (e.g., available at the website https://www.ncbi.nlm.nih.gov/nuccore/NM_000449.4).

As used herein, the terms “serpin family A member 3” and “SERPINA3” are used interchangeably to refer to a polypeptide encoded by a SERPINA3 gene. SERPINA3 is also known as alpha-1-antichymotrypsin. In some instances, the SERPINA3 is a human SERPINA3. An exemplary SERPINA3 gene is available as Gene ID: 12 (e.g., available at the website www[dot]ncbi[dot]nlm[dot]nih[dot]gov/gene/12). An exemplary SERPINA3 amino acid sequence is provided herein in as SEQ ID NO: 5. An exemplary SERPINA3 nucleotide sequence is provided herein in as SEQ ID NO: 16.

As used herein, the terms “serpin family B member 9” and “SERPINB9” are used interchangeably to refer to a polypeptide encoded by a SERPINB9 gene. SERPINB9 is also known as CAP-3; CAP3; PI-9; PI9; serine (or cysteine) proteinase inhibitor, clade B (ovalbumin), member 9; serpin peptidase inhibitor, clade B (ovalbumin), member 9; and cytoplasmic antiproteinase 3. In some instances, the SERPINB9 is a human SERPINB9. An exemplary SERPINB9 gene is available as Gene ID: 5272 (e.g., available at the website www[dot]ncbi[dot]nlm[dot]nih[dot]gov/gene/5272). An exemplary nucleotide sequence that can be used to generate SERPINB9 is available as Transcript ID NM_004155.6 (e.g., available at the website www[dot]ncbi[dot]nlm[dot]nih[dot]gov/nuccore/NM_004155.6), as well as the nucleotide sequence provided herein in as SEQ ID NO: 17. An exemplary SERPINB9 amino acid sequence is available as Protein ID NP_004146.1 (e.g., available at the website www[dot]ncbi[dot]nlm[dot]nih[dot]gov/protein/NP_004146.1), as well as the amino acid sequence provided herein in as SEQ ID NO: 6.

As used herein, the term “stem cell” refers to a cell with the ability to divide for indefinite periods in culture and to give rise to specialized cells.

As used herein, the term “substantially” or “essentially” refers to a quantity, level, value, number, frequency, percentage, dimension, size, amount, weight or length that is about 90%, 91%, 92%, 93%, 94%, 95%, 96%, 97%, 98%, or 99% or higher compared to a reference quantity, level, value, number, frequency, percentage, dimension, size, amount, weight or length. In some instances, the terms “essentially the same” or “substantially the same” refer a range of quantity, level, value, number, frequency, percentage, dimension, size, amount, weight or length that is about the same as a reference quantity, level, value, number, frequency, percentage, dimension, size, amount, weight or length.

As used herein, the term “sustained transcriptionally active payload region” or “STAPLR” and its grammatical equivalents, refers to an intergenic region in the mammalian genome that allows consistent levels of expression of transgenes integrated therein, including as the cell undergoes changes in its differentiation state. STAPLR expands the repertoire of genomic safe harbors where transgenes can be stably integrated and their expression can be maintained over multiple passages and as the cell changes its phenotype. The term “payload” or “genomic payload” in the context of STAPLR refers to exogenous or heterologous nucleotide sequences introduced to the region. The STAPLR can refer to an intergenic region found between essential genes or genes that are expressed throughout different cell states. Additional information on STAPLR are provided in the international patent application published as WO2023212722A1, which is incorporated herein by reference.

As used herein, the term “sustained transgene expression locus” or “STEL” refer to a locus in the genome of an organism that is resistant to silencing of gene expression. For instance, a STEL can be resistant to silencing over time or after changes in cell fate (e.g., differentiation), such that expression of genes contained in the STEL is sustained. Exemplary STEL include, but are not limited to genes encoding ribosomal subunits, mitochondria proteins, actin proteins, eukaryotic translation factors, and histones. Additional exemplary STEL are described in WO2021072329A1, which is incorporated herein by reference.

As used herein, the term “T cell” refers to a type of lymphocyte that plays a central role in cell-mediated immunity. T cells may be distinguished from other lymphocytes, such as B cells and natural killer cells (NK cells), by the presence of a T-cell receptor (TCR) on the cell surface. Exemplary T cell types include, but are not limited to, conventional adaptive T cells, which include helper CD4+ T cells (also known as helper T cells), CD8+ T cells (also known as cytotoxic T cells), memory and naive T cells of both CD4+ and CD8+ T cells, and regulatory T cells (also known as T regs), and innate-like T cells including natural killer T cells (also known as NKT cells), mucosal associated invariant T cells, and gamma delta T cells. T cells can be naturally occurring or non-natural, e.g., modified T cells, such as CAR-T cells.

As used herein, the terms “Tumor Necrosis Factor-related apoptosis-inducing ligand” and “TRAIL” are used interchangeably to refer to a polypeptide encoded by a TRAIL gene. TRAIL is also known as TNFSF10; APO2L; Apo-2L; cluster of differentiation 253; CD253; TL2; TNLG6A; tumor necrosis factor superfamily member 10; tumor necrosis factor (ligand) superfamily member 10; TNF superfamily member 10; and TANCR. In some instances, the TRAIL is a human TRAIL. An exemplary TRAIL gene is available as Gene ID: 8743 (e.g., available at the website www[dot]ncbi[dot]nlm[dot]nih[dot]gov/gene/8743). Exemplary nucleotide sequences that can be used to generate TRAIL are available as Transcript ID NM_001190942.2 (e.g., available at the website www[dot]ncbi[dot]nlm[dot]nih[dot]gov/nuccore/NM_001190942.2), Transcript ID NM_001190943.2 (e.g., available at the website https://www[dot]ncbi[dot]nlm[dot]nih[dot]gov/nuccore/NM_001190943[dot]2), Transcript ID NM_003810.4 (e.g., available at the website https://www[dot]ncbi[dot]nlm[dot]nih[dot]gov/nuccore/NM_003810[dot]4), Transcript ID ENST00000420541 (e.g., available at the website https://useast[dot]ensembl[dot]org/Homo_sapiens/Transcript/Sequence_cDNA?db=core;g=E NSG00000121858;r=3:172505508-172523475;t=ENST00000420541) and Transcript ID ENST00000430881 (e.g., available at the website https://useast[dot]ensembl[dot]org/Homo_sapiens/Transcript/Sequence_cDNA?db=core;g=E NSG00000121858;r=3:172505508-172523475;t=ENST00000430881); as well as the nucleotide sequence provided herein in as SEQ ID NO: 15. Exemplary amino acid sequences of TRAIL are available as Protein ID NP_001177871.1 (e.g., available at the website www[dot]ncbi[dot]nlm[dot]nih[dot]gov/protein/NP_001177871.1), Protein ID NP_001177872.1 (e.g., available at the website https://www[dot]ncbi[dot]nlm[dot]nih[dot]gov/protein/NP_001177872[dot]1), Protein ID NP_003801.1 (e.g., available at the website https://www[dot]ncbi[dot]nlm[dot]nih[dot]gov/protein/NP_003801[dot]1), Protein ID ENST00000420541 (e.g., available at the website https://useast[dot]ensembl[dot]org/Homo_sapiens/Transcript/Sequence_Protein?db=core;g=E NSG00000121858;r=3:172505508-172523475;t=ENST00000420541), and Protein ID ENST00000430881 (e.g., available at the website https://useast[dot]ensembl[dot]org/Homo_sapiens/Transcript/Sequence_Protein?db=core;g=E NSG00000121858;r=3:172505508-172523475;t=ENST00000430881); as well as the amino acid sequence provided herein in as SEQ ID NO: 4.

As used herein, the term “treat,” or a grammatical equivalent thereof, refers to a means to reduce the frequency or severity of at least one sign or symptom of a disease or disorder experienced by a subject.

As used herein, the term “variant” when used in the context of a polypeptide, protein, nucleic acid, or polynucleotide refers to a polypeptide, protein, nucleic acid, or polynucleotide that includes at least one alteration (e.g., a substitution, a deletion, or an addition of one or more amino acid or nucleotide) relative to the amino acid sequence of a parent polypeptide or protein (e.g., endogenous polypeptide) or the nucleotide sequence of a parent nucleic acid or polynucleotide (e.g., endogenous gene), but the resulting variant polypeptide, protein, nucleic acid, or polynucleotide retains substantially the same function as the parent a polypeptide, protein, nucleic acid, or polynucleotide. The parent sequence of amino acids or nucleic acids can be, for example, a wild-type sequence or a homolog thereof, or a variant of a wild-type sequence or homolog thereof.

As used herein, the terms “V-domain Ig suppressor of T cell activation” and “VISTA” are used interchangeably to refer to a polypeptide encoded by a VISTA gene. VISTA is also known as VSIR; B7-H5; B7H5; GI24; PP2135; SISP1; DD1alpha; VISTA; C10orf54; chromosome 10 open reading frame 54; PD-1H; V-set immunoregulatory receptor; Dies1; PDCD1 homolog; stress induced secreted protein 1; and SISP1. In some instances, the VISTA is a human VISTA. An exemplary VISTA gene is available as Gene ID: 64115 (e.g., available at the website www[dot]ncbi[dot]nlm[dot]nih[dot]gov/gene/64115). An exemplary nucleotide sequence that can be used to generate VISTA is available as Transcript ID NM_022153.2 (e.g., available at the website www[dot]ncbi[dot]nlm[dot]nih[dot]gov/nuccore/NM_022153.2), as well as the nucleotide sequence provided herein in as SEQ ID NO: 12. An exemplary VISTA amino acid sequence is available as Protein ID NP_071436.1 (e.g., available at the website www[dot]ncbi[dot]nlm[dot]nih[dot]gov/protein/NP_071436.1), as well as the amino acid sequence provided herein in as SEQ ID NO: 1.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### II. Engineered Cells

Certain aspects of the disclosure provide an engineered cell. In some embodiments, the engineered cell includes at least one heterologous nucleic acid sequence encoding a polypeptide or a combination of peptides. In some embodiments, the polypeptide or combination of peptides are expressed at levels sufficient to inhibit natural killer (NK) cell-mediated cytotoxicity.

In some embodiments, the polypeptide includes one or more of VISTA, CLEC2D, TRAIL, SERPINB9, HLA-C, and CD47 or a variant thereof. In some embodiments, the polypeptide includes one or more of CLEC2D, TRAIL, SERPINB9, and HLA-C or a variant thereof. In some embodiments, the combination of polypeptides includes two or more of VISTA, CLEC2D, TRAIL, SERPINB9, HLA-C, and CD47 or a variant thereof. In some embodiments, the combination of polypeptides includes two or more of CLEC2D, TRAIL, SERPINB9, and HLA-C or a variant thereof. In some embodiments, the combination of polypeptides includes two or more of CLEC2D, TRAIL, and SERPINB9, or a variant thereof. In some embodiments, the combination of polypeptides includes three or more of VISTA, CLEC2D, TRAIL, SERPINB9, HLA-C, and CD47 or a variant thereof. In some embodiments, the combination of polypeptides includes three or more of CLEC2D, TRAIL, SERPINB9, and HLA-C or a variant thereof. In some embodiments, the combination of polypeptides includes CLEC2D, TRAIL, and SERPINB9, or a variant thereof. In some embodiments, the combination of polypeptides includes four or more of VISTA, CLEC2D, TRAIL, SERPINB9, HLA-C, and CD47 or a variant thereof. In some embodiments, the combination of polypeptides includes CLEC2D, TRAIL, SERPINB9, and HLA-C, or a variant thereof. In some embodiments, the combination of polypeptides includes five or more of VISTA, CLEC2D, TRAIL, SERPINB9, HLA-C, and CD47 or a variant thereof. In some embodiments, the combination of polypeptides includes VISTA, CLEC2D, TRAIL, SERPINB9, HLA-C, and CD47 or a variant thereof. In some embodiments, the combination of polypeptides includes one or more of VISTA, CLEC2D, TRAIL, and SERPINB9 or a variant thereof. In some embodiments, the combination of polypeptides includes CLEC2D or a variant thereof and TRAIL or a variant thereof. In some embodiments, the combination of polypeptides includes CLEC2D or a variant thereof and SERPINB9 or a variant thereof. In some embodiments, the polypeptide includes VISTA or a variant thereof. In some embodiments, the polypeptide includes human CLEC2D or a variant thereof. In some embodiments, the polypeptide includes human TRAIL or a variant thereof. In some embodiments, the polypeptide includes human SERPINB9 or a variant thereof. In some embodiments, the polypeptide includes human HLA-C or a variant thereof. In some embodiments, the polypeptide includes human CD47 or a variant thereof.

In some embodiments, the polypeptide, including a variant of a polypeptide described herein, includes an amino acid sequence that has at least 65%, at least 70%, at least 75%, at least 80%, at least 85%, at least 90%, at least 95%, at least 96%, at least 97%, at least 98%, or at least 99% identity to any one of SEQ ID NOS: 1-11. In some embodiments, the polypeptide includes an amino acid sequence that has at least 65% identity to any one of SEQ ID NOS: 1-11. In some embodiments, the polypeptide includes an amino acid sequence that has at least 70% identity to any one of SEQ ID NOS: 1-11. In some embodiments, the polypeptide includes an amino acid sequence that has at least 75% identity to any one of SEQ ID NOS: 1-11. In some embodiments, the polypeptide includes an amino acid sequence that has at least 80% identity to any one of SEQ ID NOS: 1-11. In some embodiments, the polypeptide includes an amino acid sequence that has at least 85% identity to any one of SEQ ID NOS: 1-11. In some embodiments, the polypeptide includes an amino acid sequence that has at least 90% identity to any one of SEQ ID NOS: 1-11. In some embodiments, the polypeptide includes an amino acid sequence that has at least 95% identity to any one of SEQ ID NOS: 1-11. In some embodiments, the polypeptide includes an amino acid sequence that has at least 98% identity to any one of SEQ ID NOS: 1-11. In some embodiments, the polypeptide includes an amino acid sequence that has at least 99% identity to any one of SEQ ID NOS: 1-11. In some embodiments, the polypeptide includes an amino acid sequence that is an identical sequence to any one of SEQ ID NOS: 1-11.

In some embodiments, the polypeptide, including a variant of a polypeptide described herein, includes an amino acid sequence that has at least 65%, at least 70%, at least 75%, at least 80%, at least 85%, at least 90%, at least 95%, at least 96%, at least 97%, at least 98%, or at least 99% identity to any one of SEQ ID NOS: 2, 4, 5, and 11. In some embodiments, the polypeptide includes an amino acid sequence that has at least 65% identity to any one of SEQ ID NOS: 2, 4, 5, and 11. In some embodiments, the polypeptide includes an amino acid sequence that has at least 70% identity to any one of SEQ ID NOS: 2, 4, 5, and 11. In some embodiments, the polypeptide includes an amino acid sequence that has at least 75% identity to any one of SEQ ID NOS: 2, 4, 5, and 11. In some embodiments, the polypeptide includes an amino acid sequence that has at least 80% identity to any one of SEQ ID NOS: 2, 4, 5, and 11. In some embodiments, the polypeptide includes an amino acid sequence that has at least 85% identity to any one of SEQ ID NOS: 2, 4, 5, and 11. In some embodiments, the polypeptide includes an amino acid sequence that has at least 90% identity to any one of SEQ ID NOS: 2, 4, 5, and 11. In some embodiments, the polypeptide includes an amino acid sequence that has at least 95% identity to any one of SEQ ID NOS: 2, 4, 5, and 11. In some embodiments, the polypeptide includes an amino acid sequence that has at least 98% identity to any one of SEQ ID NOS: 2, 4, 5, and 11. In some embodiments, the polypeptide includes an amino acid sequence that has at least 99% identity to any one of SEQ ID NOS: 2, 4, 5, and 11. In some embodiments, the polypeptide includes an amino acid sequence that is an identical sequence to any one of SEQ ID NOS: 2, 4, 5, and 11.

In some embodiments, the polypeptide, including a variant of a polypeptide described herein, includes immune evasion activity. For instance, in some embodiments, the polypeptide or a variant of the polypeptide, when expressed by a cell at a suitable level, results in immune evasion activity. In some embodiments, the immune evasion activity includes inhibition of natural killer cell mediated cytotoxicity by the engineered cell. In some embodiments, the immune evasion activity include reduced T cell mediated killing of the engineered cell. In some embodiments, a polypeptide having at least 65%, at least 70%, at least 75%, at least 80%, at least 85%, at least 90%, at least 95%, at least 96%, at least 97%, at least 98%, or at least 99% identity to any one of SEQ ID NOS: 1-11, includes the immune evasion activity. In some embodiments, a polypeptide having at least 65%, at least 70%, at least 75%, at least 80%, at least 85%, at least 90%, at least 95%, at least 96%, at least 97%, at least 98%, or at least 99% identity to any one of SEQ ID NOS: 2, 4, 5, and 11, includes the immune evasion activity.

In some embodiments, the engineered cell that includes at least one heterologous nucleic acid sequence encoding a polypeptide or a combination of peptides, or a variant of the polypeptide(s), includes a biological activity of said polypeptide(s). For instance, in some embodiments, the engineered cell that includes at least one heterologous nucleic acid sequence encoding any one of VISTA, CLEC2D, TRAIL, SERPINB9, HLA-C, and CD47 includes the biological activity associated with VISTA, CLEC2D, TRAIL, SERPINB9, HLA-C, and CD47, respectively. In some embodiments, the engineered cell that includes at least one heterologous nucleic acid sequence encoding a variant of VISTA, CLEC2D, TRAIL, SERPINB9, HLA-C, or CD47 includes the biological activity of VISTA, CLEC2D, TRAIL, SERPINB9, HLA-C, or CD47, respectively. In some embodiments, the engineered cell that includes at least one heterologous nucleic acid sequence encoding a polypeptide or a combination of polypeptides having at least 65%, at least 70%, at least 75%, at least 80%, at least 85%, at least 90%, at least 95%, at least 96%, at least 97%, at least 98%, or at least 99% identity to any one of SEQ ID NOS: 1-11, includes the biological activity associated with a polypeptide having an identical sequence to any one of SEQ ID NOS: 1-11, respectfully. In some embodiments, the engineered cell that includes at least one heterologous nucleic acid sequence encoding a polypeptide or a combination of polypeptides having at least 65%, at least 70%, at least 75%, at least 80%, at least 85%, at least 90%, at least 95%, at least 96%, at least 97%, at least 98%, or at least 99% identity to any one of SEQ ID NOS: 2, 4, 5, and 11, includes the biological activity associated with a polypeptide having an identical sequence to any one of SEQ ID NOS: 2, 4, 5, and 11, respectfully.

In some embodiments, the engineered cell includes at least two different heterologous nucleic acid sequences, each encoding a distinct polypeptide. In some embodiments, the engineered cell includes at least three different heterologous nucleic acid sequences, each encoding a distinct polypeptide. In some embodiments, the engineered cell includes at least four different heterologous nucleic acid sequences, each encoding a distinct polypeptide. In some embodiments, the engineered cell includes at least five different heterologous nucleic acid sequences, each encoding a distinct polypeptide. In some embodiments, the engineered cell includes at least six different heterologous nucleic acid sequences, each encoding a distinct polypeptide.

In some embodiments, the engineered cell includes at least two different heterologous nucleic acid sequences, each encoding CLEC2D or a variant thereof and SERPINB9 or a variant thereof. In some embodiments, the engineered cell includes at least two different heterologous nucleic acid sequences, each encoding CLEC2D or a variant thereof and TRAIL or a variant thereof. In some embodiments, the engineered cell includes at least two different heterologous nucleic acid sequences, each encoding CLEC2D or a variant thereof and HLA-C or a variant thereof. In some embodiments, the engineered cell includes at least two different heterologous nucleic acid sequences, each encoding TRAIL or a variant thereof and SERPINB9 or a variant thereof. In some embodiments, the engineered cell includes at least two different heterologous nucleic acid sequences, each encoding TRAIL or a variant thereof and HLA-C or a variant thereof. In some embodiments, the engineered cell includes at least two different heterologous nucleic acid sequences, each encoding SERPINB9 or a variant thereof and HLA-C or a variant thereof.

In some embodiments, the at least one heterologous nucleic acid sequence has at least 65%, at least 70%, at least 75%, at least 80%, at least 85%, at least 90%, at least 95%, at least 96%, at least 97%, at least 98%, or at least 99% identity to any one of SEQ ID NOS: 12-22. In some embodiments, the at least one heterologous nucleic acid sequence has at least 65% identity to any one of SEQ ID NOS: 12-22. In some embodiments, the at least one heterologous nucleic acid sequence has at least 75% identity to any one of SEQ ID NOS: 12-22. In some embodiments, the at least one heterologous nucleic acid sequence has at least 85% identity to any one of SEQ ID NOS: 12-22. In some embodiments, the at least one heterologous nucleic acid sequence has at least 90% identity to any one of SEQ ID NOS: 12-22. In some embodiments, the at least one heterologous nucleic acid sequence has at least 95% identity to any one of SEQ ID NOS: 12-22. In some embodiments, the at least one heterologous nucleic acid sequence has at least 98% identity to any one of SEQ ID NOS: 12-22. In some embodiments, the at least one heterologous nucleic acid sequence has at least 99% identity to any one of SEQ ID NOS: 12-22. In some embodiments, the at least one heterologous nucleic acid sequence has an identical sequence to one of SEQ ID NOS: 12-22.

In some embodiments, the at least one heterologous nucleic acid sequence has at least 65%, at least 70%, at least 75%, at least 80%, at least 85%, at least 90%, at least 95%, at least 96%, at least 97%, at least 98%, or at least 99% identity to any one of SEQ ID NOS: 13, 15, 17 and 22. In some embodiments, the at least one heterologous nucleic acid sequence has at least 65% identity to any one of SEQ ID NOS: 13, 15, 17 and 22. In some embodiments, the at least one heterologous nucleic acid sequence has at least 75% identity to any one of SEQ ID NOS: 13, 15, 17 and 22. In some embodiments, the at least one heterologous nucleic acid sequence has at least 85% identity to any one of SEQ ID NOS: 13, 15, 17 and 22. In some embodiments, the at least one heterologous nucleic acid sequence has at least 90% identity to any one of SEQ ID NOS: 13, 15, 17 and 22. In some embodiments, the at least one heterologous nucleic acid sequence has at least 95% identity to any one of SEQ ID NOS: 13, 15, 17 and 22. In some embodiments, the at least one heterologous nucleic acid sequence has at least 98% identity to any one of SEQ ID NOS: 13, 15, 17 and 22. In some embodiments, the at least one heterologous nucleic acid sequence has at least 99% identity to any one of SEQ ID NOS: 13, 15, 17 and 22. In some embodiments, the at least one heterologous nucleic acid sequence has an identical sequence to one of SEQ ID NOS: 13, 15, 17 and 22.

In some embodiments, the at least one heterologous nucleic acid sequence is integrated into a genomic locus. In some embodiments, the at least one heterologous nucleic acid sequence is integrated into a sustained transgene expression locus (STEL) in the genome of the engineered cell. In some embodiments, the STEL is a gene locus that encodes a protein involved in one or more of: ribonucleoprotein complex formation, focal adhesion, cell-substrate adherens junction, cell-substrate junction, cell anchoring, extracellular exosome, extracellular vesicle, intracellular organelle, anchoring junction, RNA binding, nucleic acid binding (e.g., rRNA or mRNA binding), and protein binding. In some embodiments, the STEL is a glyceraldehyde 3-phosphate dehydrogenase (GAPDH) gene. In some embodiments, the STEL is a ribosomal protein gene locus, such as an RPL or RPS gene locus. Examples of RPL genes are RPL10, RPL13, RPS18, RPL3, RPLPI, RPL13A, RPL15, RPL41, RPL11, RPL32, RPL18A, RPL19, RPL28, RPL29, RPL9, RPL8, RPL6, RPL 18, RPL7, RPL7A, RPL21, RPL37A, RPL12, RPL5, RPL34, RPL35A, RPL30, RPL24, RPL39, RPL37, RPL14, RPL27A, RPLP2, RPLP0, RPL23A, RPL26, RPL36, RPL35, RPL23, RPL4, and RPL22. Examples of RPS genes are RPS2, RPS 19, RPS 14, RPS3A, RPS 12, RPS3, RPS6, RPS23, RPS27A, RPS8, RPS4X, RPS7, RPS24, RPS27, RPS15A, RPS9, RPS28, RPS 13, RPSA, RPS5, RPS16, RPS25, RPS15, RPS20, and RPSH. In some embodiments, the STEL is a gene locus encoding a mitochondrial protein, such as MT-COl, MT-C02, MT-ND4, MT-ND1, and MT-ND2. In some embodiments, the STEL is a gene locus encoding an actin protein, such as ACTG1 and ACTB. In some embodiments, the STEL is a gene locus encoding a eukaryotic translation elongation factor, such as EEF1A1 and EEF2, or a eukaryotic translation initiation factor such as EIEI. In some embodiments, the STEL is a gene locus encoding a histone, such as H3F3A and H3F3B. In some embodiments, the STEL is a gene locus selected from FTL, FTH1, TPT1, IMSB10, GAPDH, PTMA, GNB2L1, NACA, YBX1, NPM1, FAU, UBA52, HSP90AB1, MYL6, SERF2, and SRP14. Compositions and methods for integrating the heterologous nucleic acid into a STEL are described in International Patent Application Publication Nos. WO 2021/072329 and WO 2024/145653, which are incorporated by reference.

Advantageously, by integrating the at least one heterologous nucleic acid encoding the polypeptide into a STEL in the genome of the cells, the polypeptide encoded by the at least one heterologous nucleic acid can be governed by an endogenous gene promoter, such as a GAPDH promoter. Consequently, the expression of the polypeptide can be linked to an endogenous gene's expression. The continued activity of the endogenous gene in the engineered cells will then imply that the expression of the polypeptide will remain sustained and constitutive. By leveraging the inherent regulatory mechanisms of the endogenous gene, the disclosure offers a useful mechanism to maintain expression of the polypeptide at a level sufficient to inhibit natural killer cell mediated cytotoxicity.

In some embodiments, the heterologous nucleic acid is integrated in the genome of the engineered cell in a sustained transcriptionally active payload region (STAPLR). In some embodiments, the STAPLR is selected from the group consisting of: the intergenic region between the RPL34 gene and the OSTC gene; the intergenic region between the ACTB gene and the FSCN1 gene; the intergenic region between the AKIRIN1 gene and the NDUFS5 gene; the intergenic region between the PRDX1 gene and the AKR1A1 gene; the intergenic region between the PTGES3 gene and the NACA gene; the intergenic region between the MLF2 gene and the PTMS gene; the intergenic region between the RAB13 gene and the RPS27 gene; the intergenic region between the JTB gene and the RAB13 gene; the intergenic region between the AKR1A1 gene and the NASP gene; the intergenic region between the NDUFS5 gene and the MACF1 gene; the intergenic region between the SRSF9 gene and the DYNLL1 gene; the intergenic region between the MYL6B gene and the MYL6 gene; the intergenic region between the GPX1 gene and the RHOA gene; the intergenic region between the HNRNPA2B1 gene and the CBX3 gene; the intergenic region between the ROMO gene and the RBM39 gene; and the intergenic region between the PA2G4 gene and the RPL41 gene. In some embodiments, the STAPLR is the intergenic region between the PRDX1 gene and the AKR1A1 gene. In some embodiments, the heterologous nucleic acid is integrated at a location that is at least 100-5000 base pairs away from the nearest gene. Compositions and methods for integrating a heterologous nucleic acid into a STAPLR are described in International Patent Application Publication No. WO 2024/145653, which are incorporated by reference.

In some embodiments, the engineered cell includes a kill switch. The inclusion of a kill switch can be used to increase the safety of the engineered cell. For instance, activation of the kill switch can remove the engineered cells without interfering with a subject's own cells. In some embodiments, the kill switch is a gene that encodes a herpes simplex virus thymidine kinase (HSV-TK), an inducible caspase9 (also known as incasep 9, and iCasp9), CD20, or a mutant human thymidylate kinase (mTMPK). In some embodiments, the kill switch is under the control of an inducible promoter. In some embodiments, the kill switch is encoded by a heterologous nucleic acid. In some embodiments, the at least one heterologous nucleic acid encoding the kill switch integrated into a different locus than the at least one heterologous nucleic acid encoding the polypeptide. In some embodiments, the at least one heterologous nucleic acid encoding the kill switch integrated into the same locus as the at least one heterologous nucleic acid encoding the polypeptide.

In some embodiments, the engineered cell further includes a genetic modification that results in reduced T cell mediated killing as compared to a wild-type cell. In some embodiments, the engineered cell further includes a genetic modification that results in at least 10%, at least 20%, at least 30%, at least 40%, at least 50%, at least 60%, at least 70%, at least 80%, at least 90%, at least 95%, or at least 100% reduced T cell mediated killing as compared to a wild-type cell. In some embodiments, the engineered cell further includes a genetic modification that results in at least 20% reduced T cell mediated killing as compared to a wild-type cell. In some embodiments, the engineered cell further includes a genetic modification that results in at least 50% reduced T cell mediated killing as compared to a wild-type cell. In some embodiments, the engineered cell further includes a genetic modification that results in at least 80% reduced T cell mediated killing as compared to a wild-type cell. In some embodiments, the engineered cell further includes a genetic modification that results in at least 90% reduced T cell mediated killing as compared to a wild-type cell. In some embodiments, the engineered cell further includes a genetic modification that results in at least 95% reduced T cell mediated killing as compared to a wild-type cell. In some embodiments, the engineered cell further includes a genetic modification that results in at least 100% reduced T cell mediated killing as compared to a wild-type cell.

In some embodiments, the genetic modification that results in reduced T cell mediated killing of the engineered cell as compared to a wild-type cell includes a deletion, disruption, or attenuation of a β2 microglobulin (B2M) gene. In some embodiments, the genetic modification that reduces T cell mediated killing of the engineered cell as compared to a wild-type cell includes a deletion, disruption, or attenuation of one or more of an HLA-A gene, an HLA-B gene, and an HLA-C gene.

In some embodiments, the engineered cell further includes a deletion, disruption, or attenuation of a class II, major histocompatibility complex, transactivator (CIITA) gene or a regulatory factor 5 (RFX5) gene. Accordingly, in some embodiments, the engineered cell includes a genetic modification that results in reduced T cell mediated killing of the engineered cell as compared to a wild-type cell as described herein (e.g., a deletion, disruption, or attenuation of a B2M, HLA-A, HLA-B, and/or HLA-C) and a deletion, disruption, or attenuation of the CIITA gene. In some embodiments, the engineered cell includes a genetic modification that results in reduced T cell mediated killing of the engineered cell as compared to a wild-type cell as described herein (e.g., a deletion, disruption, or attenuation of a B2M, HLA-A, HLA-B, and/or HLA-C) and a deletion, disruption, or attenuation of the RFX5 gene. In some embodiments, the engineered cell includes a genetic modification that results in reduced T cell mediated killing of the engineered cell as compared to a wild-type cell as described herein (e.g., a deletion, disruption, or attenuation of a B2M, HLA-A, HLA-B, and/or HLA-C) and a deletion, disruption, or attenuation of the CIITA gene and the RFX5 gene.

In some embodiments, the engineered cell further includes at least one selectable marker gene. The selectable marker can be used for detection or selection of the engineered cell, and its expression can be detected by any suitable method known in the art, such as for example, FACS or culturing in a selection media. Non-limiting examples of selectable marker genes include, but are not limited to, fluorescent proteins (such as green fluorescent protein (GFP), blue fluorescent protein (EBFP, EBFP2, Azurite, mKalamal), cyan fluorescent protein (ECFP, Cerulean, CyPet, mTurquoise2), and yellow fluorescent protein derivatives (YFP, Citrine, Venus, YPet, EYFP), b-galactosidase (LacZ), chloramphenicol acetyltransferase (cat), neomycin phosphotransferase (neo), puromycin N-acetyl-transferase (PAC), enzymes (such as oxidases and peroxidases), and antigenic molecules. In some embodiments, the selectable marker gene can be driven by an endogenous gene promoter.

In some embodiments, the engineered cell includes an engineered stem cell. In some embodiments, the engineered stem cell includes a pluripotent stem cell. In some embodiments, the pluripotent stem cell includes an embryonic stem cell (ESC) or induced pluripotent stem cell (iPSC). In some embodiments, the engineered stem cell includes a multipotent stem cell.

In some embodiments, the engineered cell includes a cardiac cell, a neural cell, a T cell (e.g., a regulator T cell), a retinal cell, an endocrine cell, an epithelial cell, a muscle cell, or a myeloid cell. In some embodiments, the engineered cell includes a cardiac cell. In some embodiments, the cardiac cell is a cell of the epicardium, the myocardium, or the endocardium of the heart. In some embodiments, the cardiac cell is a cardiomyocyte, a cardiac fibroblast, a cardiac smooth muscle cell, an epicardium cells, a cardiac endothelial cell, a Purkinje fiber, or a pacemaker cell. In some embodiments, the engineered cell includes a neural cell. In some embodiments, the neural cell is a neuron or a glial cell. In some embodiments, the engineered cell includes a myeloid cell. In some embodiments, the myeloid cell is a monocyte, a microglia, a macrophage, a dendritic cell, a basophil, an eosinophil, an erythrocyte, a mast cell, a neutrophil, a megakaryocyte, or a platelet, or any precursor progenitor cell thereof. In some embodiments, the cardiac cell, the neural cell, or the myeloid cell are derived from an engineered stem cell (e.g., an engineered stem cell described herein). In some embodiments, the T cell is a helper T cell (CD4+ T cell), a cytotoxic T cell (CD8+ T cell), a memory T cell, a regulatory T cell (T reg cell), an innate-like T cell (e.g., a natural killer T cell), a mucosal-associated invariant T cell, or a gamma delta T cell, or a modified T cell (e.g., a CAR-T cell). In some embodiments, the retinal cell is a photoreceptor, a retinal horizontal cell, a retinal bipolar cell, a retinal amacrine cell, or a retinal ganglion cell. In some embodiments, the endocrine cell is a hypothalamus endocrine cell a pituitary gland endocrine cell, a pineal gland endocrine cell, a thyroid gland endocrine cell (e.g., a follicular cell), a parathyroid gland endocrine cell, a thymus gland endocrine cell, an adrenal gland endocrine cell, a pancreatic endocrine cell (e.g., an alpha cell, a beta cell, a delta cell or an F cell), an ovarian endocrine cell (e.g., a granulosa cell), or a testicular endocrine cell (e.g., a Leydig cell). In some embodiments, the epithelial cell is a squamous epithelial cell, a cuboidal epithelial cell, a columnar epithelial cell, a pseudostratified epithelia cell, or a stratified epithelial cell. In some embodiments, the muscle cell is a skeletal muscle cell, a cardiac muscle cell (e.g., a cardiomyocyte), or a smooth muscle cell.

In some embodiments, the engineered cell is a human engineered cell.

Accordingly, in one aspect, this disclosure provides cells, and methods thereof, that have been engineered to reduce or inhibit cytotoxicity mediated by natural killer (NK) cells, focusing on targeting genes involved in specific cell-to-NK cell interactions. In certain embodiments, the engineering strategy is designed to upregulate the expression of proteins that interact with receptors on NK cells, such as CD161. For instance, one insight of this disclosure is that the gene CLEC2D, when overexpressed, can modulate NK cell activity through its binding to CD161 on these cells, resulting in a reduced rate of NK cell-induced killing.

In another aspect, this disclosure provides cells, and methods thereof, that have been engineered to inhibit or reduce cytotoxicity mediated by natural killer (NK) cells by modulating the expression of one or more proteins involved in apoptosis. In some embodiments, this disclosure provides cells that have been engineered to overexpress one or more of SERPINB9, SERPINA3, TRAIL, or FASL. For example, in some embodiments, this disclosure provides cells that have been engineered to increase the expression of TRAIL, providing the cells with an ability to induce apoptosis in target cells through the ligation to TRAIL receptors, specifically TRAIL-R1 or TRAIL-R2. This strategic increase in TRAIL expression serves as a countermeasure against the cytotoxic actions of NK cells, thereby potentially improving the survival and efficacy of these engineered cells in therapeutic applications where NK cell activity poses a significant challenge.

In another aspect, this disclosure provides cells, and methods thereof, that have been engineered to inhibit or reduce cytotoxicity mediated by natural killer (NK) cells by modulating the expression of one or more proteins involved in the serine protease inhibitor family. For example, in some embodiments this disclosure provides cells that have been engineered to overexpress a protein that belongs to a subfamily of intracellular serpins that inhibits the activity of Granzyme B (GrzB), which is a mechanism by which T and NK cell kill target cells. Accordingly, in some embodiments, this disclosure provides cells that have been engineered to overexpress SERPINB9.

### Population of Cells

In some aspects of the disclosure, provided herein are populations of engineered cells (e.g., a population of the engineered cells described herein).

In some embodiments, the population of engineered cells is less susceptible to natural killer cell mediated cytotoxicity as compared to a population of cells not engineered to have the at least one heterologous nucleic acid sequence. The susceptibility of the population of engineered cells to natural killer cell mediate cytotoxicity can be measured by any suitable method known in the art. For instance, the susceptibility to natural killer cell mediate cytotoxicity may be measured by a natural killer cell cytotoxicity assay, e.g., a natural killer cell cytotoxicity assay as described in Example 2. In some embodiments, the population of engineered cells is at least about 10%, at least about 20%, at least about 30%, at least about 40%, at least about 50%, at least about 60%, at least about 70%, at least about 80%, or at least about 90% less susceptible to natural killer cell mediated cytotoxicity as compared to a population of cells not engineered to have the at least one heterologous nucleic acid sequence. In some embodiments, the population of engineered cells is at least about 10% less susceptible to natural killer cell mediated cytotoxicity as compared to a population of cells not engineered to have the at least one heterologous nucleic acid sequence. In some embodiments, the population of engineered cells is at least about 20% less susceptible to natural killer cell mediated cytotoxicity as compared to a population of cells not engineered to have the at least one heterologous nucleic acid sequence. In some embodiments, the population of engineered cells is at least about 30% less susceptible to natural killer cell mediated cytotoxicity as compared to a population of cells not engineered to have the at least one heterologous nucleic acid sequence. In some embodiments, the population of engineered cells is at least about 50% less susceptible to natural killer cell mediated cytotoxicity as compared to a population of cells not engineered to have the at least one heterologous nucleic acid sequence.

In some embodiments, the engineered cells in the population of engineered cells are positive for cardiac troponin T (cTNT). In some embodiments, at least 50%, at least 55%, at least 60%, at least 65%, at least 70%, at least 75%, at least 80%, at least 85%, at least 90%, at least 95%, at least 98%, or at least 99% of the engineered cells in the population of engineered cells are positive for cTnT. In some embodiments, at least 70% of the engineered cells in the population of engineered cells are positive for cTnT. In some embodiments, at least 80% of the engineered cells in the population of engineered cells are positive for cTnT. In some embodiments, at least 90% of the engineered cells in the population of engineered cells are positive for cTnT. In some embodiments, at least 95% of the engineered cells in the population of engineered cells are positive for cTnT. In some embodiments, at least 98% of the engineered cells in the population of engineered cells are positive for cTnT. In some embodiments, at least 99% of the engineered cells in the population of engineered cells are positive for cTnT.

In some embodiments, the population of engineered cells is present in a composition formulated for administration to a subject. The population of engineered cells can be formulated for any suitable means of administration, such as for administration by local injection into a tissue.

### Preparation of Engineered Cells

In some aspects of the disclosure, provided herein are methods of preparing any of the engineered cells described herein.

In some embodiments, the method to prepare an engineered cell described herein includes genetically modifying a cell to increase the expression of a polypeptide or a combination of polypeptides. In some embodiments, the polypeptide includes one or more of CLEC2D, TRAIL, SERPINB9, and HLA-C or a variant thereof. In some embodiments, the combination of polypeptides includes two or more of VISTA, CLEC2D, TRAIL, SERPINB9, HLA-C, and CD47 or a variant thereof. In some embodiments, the combination of polypeptides includes two or more of CLEC2D, TRAIL, SERPINB9, and HLA-C or a variant thereof. In some embodiments, the combination of polypeptides includes two or more of CLEC2D, TRAIL, and SERPINB9, or a variant thereof. In some embodiments, the combination of polypeptides includes three or more of VISTA, CLEC2D, TRAIL, SERPINB9, HLA-C, and CD47 or a variant thereof. In some embodiments, the combination of polypeptides includes three or more of CLEC2D, TRAIL, SERPINB9, and HLA-C or a variant thereof. In some embodiments, the combination of polypeptides includes CLEC2D, TRAIL, and SERPINB9, or a variant thereof. In some embodiments, the combination of polypeptides includes four or more of VISTA, CLEC2D, TRAIL, SERPINB9, HLA-C, and CD47 or a variant thereof. In some embodiments, the combination of polypeptides includes CLEC2D, TRAIL, SERPINB9, and HLA-C, or a variant thereof. In some embodiments, the combination of polypeptides includes five or more of VISTA, CLEC2D, TRAIL, SERPINB9, HLA-C, and CD47 or a variant thereof. In some embodiments, the combination of polypeptides includes VISTA, CLEC2D, TRAIL, SERPINB9, HLA-C, and CD47 or a variant thereof. In some embodiments, the combination of polypeptides includes one or more of VISTA, CLEC2D, TRAIL, and SERPINB9 or a variant thereof. In some embodiments, the combination of polypeptides includes CLEC2D or a variant thereof and TRAIL or a variant thereof. In some embodiments, the combination of polypeptides includes CLEC2D or a variant thereof and SERPINB9 or a variant thereof. In some embodiments, the polypeptide includes VISTA or a variant thereof. In some embodiments, the polypeptide includes CLEC2D or a variant thereof.

In some embodiments, genetically modifying the cell includes integrating a heterologous nucleic acid sequence encoding the polypeptide into the genome of the cell. In some embodiments, the genetically modified cell exhibits increased survival when challenged with natural killer cells as compared to a wild-type cell.

In some embodiments, the at least one heterologous nucleic acid sequence encoding the polypeptide is integrated into the genome of the cell. Any suitable technique for integrating nucleic acids into the genome of a host cell can be used. For instance, at least one heterologous nucleic acid sequence can be integrated into the genome of the cell using, for example, known gene editing systems such as those utilizing genome-targeting elements, including a DNA-binding domain (e.g., zinc finger DNA-binding protein or a TALE DNA-binding domain), guide RNA elements (e.g., CRISPR guide RNA), and guide DNA elements (e.g., NgAgo guide DNA). Programmable gene-targeting and nuclease elements enable precise targeted integration of the heterologous nucleic acid. In some instances, the at least one heterologous nucleic acid is integrated into the genome of the cell using a meganuclease based system, a zinc finger nuclease (ZFN) based system, a Transcription Activator-Like Effector-based Nuclease (TALEN) based system, a CRISPR-based system, or NgAgo-based system.

In some embodiments, the at least one heterologous nucleic acid sequence encoding the polypeptide is integrated into the genome of the cell using a CRISPR/Cas system. The CRISPR/Cas system has been used for introducing genetic modifications and gene regulation in various species. Without being limited by theory, a target nucleic acid can be modified by the interaction of the CRISPR/Cas system and a sequence present in the target nucleic acid, for example, to cause cleavage (e.g., hydrolysis of one or more phosphodiester bonds) of the target nucleic acid and introduce the genetic modification. In some embodiments, the at least one heterologous nucleic acid sequence encoding the polypeptide is integrated into the genome of the cell using a CRISPR/Cas9 system or a CRISPR/Cas12 system.

In some embodiments, the at least one heterologous nucleic acid sequence encoding the polypeptide is integrated into a STEL in the genome of the cell. In some embodiments, the STEL is a gene locus that encodes a protein involved in one or more of: ribonucleoprotein complex formation, focal adhesion, cell-substrate adherens junction, cell-substrate junction, cell anchoring, extracellular exosome, extracellular vesicle, intracellular organelle, anchoring junction, RNA binding, nucleic acid binding (e.g., rRNA or mRNA binding), and protein binding. In some embodiments, the STEL is a glyceraldehyde 3-phosphate dehydrogenase (GAPDH) gene. In some embodiments, the STEL is a ribosomal protein gene locus, such as an RPL or RPS gene locus. Examples of RPL genes are RPL10, RPL13, RPS18, RPL3, RPLP1, RPL13A, RPL15, RPL41, RPL11, RPL32, RPL18A, RPL19, RPL28, RPL29, RPL9, RPL8, RPL6, RPL 18, RPL7, RPL7A, RPL21, RPL37A, RPL12, RPL5, RPL34, RPL35A, RPL30, RPL24, RPL39, RPL37, RPL14, RPL27A, RPLP2, RPLP0, RPL23A, RPL26, RPL36, RPL35, RPL23, RPL4, and RPL22. Examples of RPS genes are RPS2, RPS 19, RPS 14, RPS3A, RPS 12, RPS3, RPS6, RPS23, RPS27A, RPS8, RPS4X, RPS7, RPS24, RPS27, RPS15A, RPS9, RPS28, RPS 13, RPSA, RPS5, RPS16, RPS25, RPS15, RPS20, and RPSII. In some embodiments, the STEL is a gene locus encoding a mitochondrial protein, such as MT-COl, MT-C02, MT-ND4, MT-ND1, and MT-ND2. In some embodiments, the STEL is a gene locus encoding an actin protein, such as ACTG1 and ACTB. In some embodiments, the STEL is a gene locus encoding a eukaryotic translation elongation factor, such as EEF1A1 and EEF2, or a eukaryotic translation initiation factor such as EIEI. In some embodiments, the STEL is a gene locus encoding a histone, such as H3F3A and H3F3B. In some embodiments, the STEL is a gene locus selected from FTL, FTH1, TPT1, IMSB10, GAPDH, PTMA, GNB2L1, NACA, YBX1, NPM1, FAU, UBA52, HSP90AB1, MYL6, SERF2, and SRP14. Compositions and methods for integrating the heterologous nucleic acid into a STEL are described in International Patent Application Publication Nos. WO 2021/072329 and WO 2024/145653, which are incorporated by reference.

In some embodiments, the heterologous nucleic acid is integrated in the genome of the engineered cell in a sustained transcriptionally active payload region (STAPLR). In some embodiments, the STAPLR is selected from the group consisting of: the intergenic region between the RPL34 gene and the OSTC gene; the intergenic region between the ACTB gene and the FSCN1 gene; the intergenic region between the AKIRIN1 gene and the NDUFS5 gene; the intergenic region between the PRDX1 gene and the AKR1A1 gene; the intergenic region between the PTGES3 gene and the NACA gene; the intergenic region between the MLF2 gene and the PTMS gene; the intergenic region between the RAB13 gene and the RPS27 gene; the intergenic region between the JTB gene and the RAB13 gene; the intergenic region between the AKR1A1 gene and the NASP gene; the intergenic region between the NDUFS5 gene and the MACF1 gene; the intergenic region between the SRSF9 gene and the DYNLL1 gene; the intergenic region between the MYL6B gene and the MYL6 gene; the intergenic region between the GPX1 gene and the RHOA gene; the intergenic region between the HNRNPA2B1 gene and the CBX3 gene; the intergenic region between the ROMO gene and the RBM39 gene; and the intergenic region between the PA2G4 gene and the RPL41 gene. In some embodiments, the STAPLR is the intergenic region between the PRDX1 gene and the AKR1A1 gene. In some embodiments, the heterologous nucleic acid is integrated at a location that is at least 100-5000 base pairs away from the nearest gene. Compositions and methods for integrating a heterologous nucleic acid into a STAPLR are described in International Patent Application Publication No. WO 2024/145653, which are incorporated by reference.

In some embodiments, the at least one heterologous nucleic acid sequence encoding the polypeptide further includes one or more regulatory elements. Such a regulatory element can include a regulatory sequence, which is any DNA sequence responsible for the regulation of gene expression, such as promoters and operators. The regulatory element can be a segment of a nucleic acid molecule, which is able to increase or decreasing the expression of specific genes within an organism.

In some embodiments, the regulatory element is a promoter. A promoter is a nucleotide sequence that directs the transcription of a structural gene. In some alternatives, a promoter is in the 5′ non-coding region of a gene, proximal to the transcriptional start site of a structural gene. Sequence elements within promoters that function in the initiation of transcription are often characterized by consensus nucleotide sequences. Without being limiting, these promoter elements can include RNA polymerase binding sites, TATA sequences, CAAT sequences, differentiation-specific elements (DSEs; McGehee et al., Mol. Endocrinol. 7:551 (1993);), cyclic AMP response elements (CREs), serum response elements (SREs; Treisman et al., Seminars in Cancer Biol. 1:47 (1990); incorporated by reference in its entirety), glucocorticoid response elements (GREs), and binding sites for other transcription factors, such as CRE/ATF (O'Reilly et al., J. Biol. Chem. 267:19938 (1992); incorporated by reference in its entirety), AP2 (Ye et al., J. Biol. Chem. 269:25728 (1994); incorporated by reference in its entirety), SPI, cAMP response element binding protein (CREB; Loeken et al., Gene Expr. 3:253 (1993); hereby expressly incorporated by reference in its entirety) and octamer factors (see, in general, Watson et al., eds., Molecular Biology of the Gene, 4th ed. (The Benjamin/Cummings Publishing Company, Inc. 1987; incorporated by reference in its entirety)), and Lemaigre and Rousseau, Biochem. J. 303:1 (1994); incorporated by reference in its entirety).

In some alternatives, promoters used herein can be inducible or constitutive promoters. Without being limiting, inducible promoters can include, for example, a tamoxifen inducible promoter, tetracycline inducible promoter, or a doxocycline inducible promoter (e.g., tre) promoter. Constitutive promoters can include, for example, SV40, CMV, UBC, EFlalpha, PGK, or CAGG. Any suitable promoter known in the art for expression of a gene in a population of engineered cells as described herein can be used. In some embodiments, the at least one heterologous nucleic acid sequence encoding the polypeptide includes a promoter. In some embodiments, expression of the polypeptide is driven by an endogenous gene promoter.

The at least one heterologous nucleic acid sequence encoding the polypeptide can be introduced to a cell by any suitable method known in the art. For instance, the at least one heterologous nucleic acid can be introduced by electroporation, sonoporation, lipofection, microinjection, biolistics, virosomes, liposomes, immunoliposomes, polycation or lipid:nucleic acid conjugates, naked DNA, artificial virions, viral vector systems (e.g., retroviral, lentivirus, adenoviral, adeno-associated, vaccinia and herpes simplex virus vectors) and agent-enhanced uptake of DNA. In some embodiments, the at least one heterologous nucleic acid encoding the polypeptide can be introduced into the cell as RNA, for example, mRNA or self-replicating RNA.

In some embodiments, the at least one heterologous nucleic acid sequence encoding the polypeptide is introduced into the cell in a vector. The vector can be a plasmid, a virus, or another vector designed for introducing a nucleic acid of interest into a cell. Viral vectors include DNA and RNA viruses, which have either episomal or integrated genomes after delivery to the cell. Exemplary viral vectors that can be used include an adeno-associated virus, a lentivirus, a retrovirus, a herpes simplex virus, vaccinia, or an adenovirus. The vector is used to introduce a gene of interest into a host cell in which the vector will interact with polymerases in the cell to express the protein encoded in the vector. The vector can exist in the cell extra-chromosomally or integrated into the genome of the host cell. In some embodiments, use of a viral vector will lead to integration of the heterologous nucleic acid into the genome of the cell.

In some embodiments, the method for preparing an engineered cells described herein includes introducing a heterologous nucleic acid encoding a kill switch into the cell. In some embodiments, the kill switch is a gene that encodes a herpes simplex virus thymidine kinase (HSV-TK), an inducible caspase9 (also known as incasep 9, and iCasp9), CD20, or a mutant human thymidylate kinase (mTMPK). In some embodiments, the kill switch is under the control of an inducible promoter.

In some embodiments, the nucleic acid encoding the kill switch is integrated into the genome of the cell using the CRISPR/Cas system. The nucleic acid encoding the kill switch can be integrated into the same or a different locus as the at least one heterologous nucleic acid sequence encoding the polypeptide.

In some embodiments, the nucleic acid encoding the kill switch is introduced into the cell in a vector. The nucleic acid encoding the kill switch can be introduced into the cell in the same vector or a different vector as the at least one heterologous nucleic acid sequence encoding the polypeptide.

In some embodiments, the method for preparing an engineered cell described herein further includes introducing a genetic modification that results in reduced T cell mediated killing of the engineered cell as compared to a wild-type cell. In some embodiments, the e genetic modification results in at least 20% reduced T cell mediated killing of the engineered cell as compared to a wild-type cell. In some embodiments, the e genetic modification results in at least 50% reduced T cell mediated killing of the engineered cell as compared to a wild-type cell. In some embodiments, the e genetic modification results in at least 80% reduced T cell mediated killing of the engineered cell as compared to a wild-type cell.

In some embodiments, the engineered cells in the population of engineered cells are positive for cardiac troponin T (cTNT). In some embodiments, at least 90% of the engineered cells in the population of engineered cells are positive for cTnT. In some embodiments, at least 95% of the engineered cells in the population of engineered cells are positive for cTnT. In some embodiments, at least 98% of the engineered cells in the population of engineered cells are positive for cTnT.

In some embodiments, the genetic modification that results in reduced T cell mediated killing of the engineered cell as compared to a wild-type cell includes a deletion, disruption, or attenuation of a β2 microglobulin (B2M) gene. In some embodiments, the genetic modification that reduces T cell mediated killing of the engineered cell as compared to a wild-type cell includes a deletion, disruption, or attenuation of one or more of an HLA-A gene, an HLA-B gene, and an HLA-C gene.

In some embodiments, the method for preparing an engineered cell described herein further includes introducing a deletion, disruption, or attenuation of the CIITA gene or the RFX5 gene. Accordingly, in some embodiments, the method includes preparing an engineered cell that includes a genetic modification that results in reduced T cell mediated killing of the engineered cell as compared to a wild-type cell as described herein (e.g., a deletion, disruption, or attenuation of a B2M, HLA-A, HLA-B, and/or HLA-C) and a deletion, disruption, or attenuation of the CIITA gene. In some embodiments, the method includes preparing an engineered cell that includes a genetic modification that results in reduced T cell mediated killing of the engineered cell as compared to a wild-type cell as described herein (e.g., a deletion, disruption, or attenuation of a B2M, HLA-A, HLA-B, and/or HLA-C) and a deletion, disruption, or attenuation of the RFX5 gene. In some embodiments, the method includes preparing an engineered cell that includes a genetic modification that results in reduced T cell mediated killing of the engineered cell as compared to a wild-type cell as described herein (e.g., a deletion, disruption, or attenuation of a B2M, HLA-A, HLA-B, and/or HLA-C) and a deletion, disruption, or attenuation of the CIITA gene and the RFX5 gene.

In some embodiments, the method for preparing an engineered cell described herein includes genetically modifying a stem cell. In some embodiments, the stem cell is a pluripotent stem cell. In some embodiments, the pluripotent stem cell is an induced pluripotent stem cell (iPSC). Methods for obtaining iPSCs for use in the method for preparing an engineered cells as described herein are known in the art. For example, the iPSCs can be prepared by inducing expression of one or more genes, e.g., POU5F1/OCT4 combined with, but not restricted to, SOX2, KLF; c-MYC, NANOG, and/or LIN28/LIN28A. Reprogramming factors can be delivered by various means (e.g., viral, non-viral, RNA, DNA, or protein delivery). Alternatively, endogenous genes can be activated by using, e.g., a CRISPR/Cas system to reprogram non-pluripotent cells into iPSCs.

In some embodiments, the method for preparing an engineered cell described herein includes genetically modifying a stem cell and differentiating the stem cell into the engineered cell. Methods for inducing differentiation of stem cell into cells of various lineages are well known in the art. For example, methods for inducing differentiation of stem cells into myeloid cells or neural cells are described, for instance, in U.S. Pat. No. 11,525,119, B2 and U.S. Pat. No. 10,260,044 B1, and in Slukvin et al., J Imm. (2006) 176:2924-32; and Su et ah, Clin Cancer Res. (2008) 14(19):6207-17; WO2021072329A1; and Tseng et al., Regen Med. (2009) 4(4):513-26, the disclosures of which are incorporated herein by reference. In some embodiments, differentiating the stem cell into the engineered cell includes contacting the stem cell with one or more differentiation factors. The specific combination of differentiation factors used will depend on the desired cell type(s). In some embodiments, differentiating the stem cell into the engineered cell includes contacting the iPSCs with one or more differentiation factors that drive the commitment and/or differentiation into myeloid progenitor cells.

In some embodiments, the method for preparing an engineered cell described herein includes genetically modifying a stem cell and differentiating the genetically modified stem cell into a cardiac cell. In some embodiments, the stem cell is a pluripotent stem cell. Any suitable method known in the art for differentiating stem cells into cardiac cells can be used in connection with the methods of the disclosure. Numerous methods exist for differentiating stem cells into cardiac cells are described, in for example, Kattman et al., Cell Stem Cell (2011) 8(2):228-40, WO2016131137, WO2018098597, U.S. Pat. No. 9,453,201, WO2020227232, WO2020227232A2; and WO2021072329, the disclosures of which are incorporated herein by reference.

In some embodiments, differentiating the stem cell into a cardiac cell includes incubating the stem cell in one or more cardiac differentiation media. For example, the cardiac differentiation media can contain varying concentrations of bone-morphogenetic protein (BMP; such as BMP4) and activin (such as activin A). Titration of differentiation factor concentration can be performed to determine the optimal concentration necessary for achieving differentiation into the desired cardiac cell type. For instance, the differentiation factors can be selected to direct differentiation into cardiomyocytes. In some embodiments, differentiating the stem cells into cardiac cells involves modulating Wnt/p-catenin signaling under fully defined conditions. For example, in some embodiments, modulating of Wnt signaling can be achieved by contacting the stem cells with a WNT activator (e.g., CHIR) followed by deactivation with a small molecule, IWR1. Further information on methods of differentiating of stem cells into cardiomyocytes can be found in Lian, Xiaojun, et al. “Directed cardiomyocyte differentiation from human pluripotent stem cells by modulating Wnt/0-catenin signaling under fully defined conditions.” Nature protocols 8.1 (2013): 162-175; and Lee, Jee Hoon, et al. “Human pluripotent stem cell-derived atrial and ventricular cardiomyocytes develop from distinct mesoderm populations.” Cell stem cell 21.2 (2017): 179-194, each of which are incorporated by reference.

In some embodiments, the method for preparing an engineered cell described herein includes genetically modifying a stem cell and differentiating the genetically modified stem cell into a neural cell. In some embodiments, the stem cell is a pluripotent stem cell. Any suitable method known in the art for differentiating stem cells into neural cells can be used in connection with the methods of the disclosure. Exemplary methods for differentiating stem cells into neural cells are described, in for example, Nat Biotechnol; 27(3): 275-280; Cell Stem Cell, 28(2): 343-355; WO2016196661A1; and WO2010096496A2, the disclosures of which are incorporated herein by reference.

For many neural cell types, differentiation from the stem cell can be first directed to adopt a primitive neural cell fate through a neural induction process involving dual SMAD inhibition (Chambers et al., Nat Biotechnol. (2009) 27(3):275-80). Primitive neural cells adopt anterior characteristics, so the lack of other signals will provide anterior/forebrain cortical cells. Caudalizing signals can be blocked to prevent paracrine signals that might otherwise generate cultures with more posterior character (for example, XAV939 can block WNT and SU5402 can block FGF signals). Dorsal cortical neurons can be made by blocking SHH activation, while ventral cortical neurons can be made through SHH activation. More caudal cell types, such as serotonergic neurons or spinal motor neurons can be made by caudalizing cultures through the addition of FGF and/or WNT signals. For some neural cell types, retinoic acid (another caudalizing agent) can be added to posteriorize cultures. The production of glial cell types can generally follow the same patterning of primitive neural cells before extended culture in FGF2 and/or EGF containing medium. Peripheral nervous system cell types can follow the same general principles but with a timely WNT signal early in the differentiation process.

In some embodiments, the method for preparing an engineered cell described herein includes genetically modifying a stem cell and differentiating the genetically modified stem cell into a myeloid cell. In some embodiments, the stem cell is a pluripotent stem cell. Any suitable method known in the art for differentiating stem cells into myeloid cells can be used in connection with the methods of the disclosure. Exemplary methods for differentiating stem cells into myeloid cells are described, in for example, Muffat et. al, Nat Med. 2016 November; 22(11): 1358-1367, Pandaya et. al, Nat Neurosci. 2017 May; 20(5): 753-759, Abud et. al, Neuron 2017 Apr. 19; 94(2):278-293, Douvaras et. al, Stem Cell Reports, Volume 8, Issue 6, P1516-1524, Jun. 6, 2017, Van Wilgenburg PLOS ONE, https://doi.org/10.1371/journal.pone.007198 August 2013, Haenseler et. al, Stem Cell Reports 2017 Jun. 6; 8(6):1727-1742 and Takata et. al, Immunity 2017 Jul. 18; 47(1):183-198, the disclosures of which are incorporated herein by reference.

In some embodiments, differentiating the genetically modified stem cell into a myeloid cell includes culturing the stem cell under conditions that induce myeloid differentiation, leading to the generation of a myeloid cell. In some embodiments, the stem cell is cultured under conditions that induce myeloid differentiation, leading to the generation of a CX3CR1+ myeloid cell. In some embodiments, the stem cell is cultured under conditions that induce myeloid differentiation, leading to the generation of a CD14+ myeloid cell. In some embodiments, the stem cell is cultured under conditions that induce myeloid differentiation, leading to the generation of a CD45+/CD14+/CX3CR1+ myeloid cell.

In some embodiments, the method for preparing an engineered cell described herein includes genetically modifying a stem cell and differentiating the genetically modified stem cell into a retinal cell. In some embodiments, differentiating the genetically modified stem cell into a myeloid cell includes culturing the stem cell under conditions that induce retinal cell differentiation, leading to the generation of a retinal cell.

In some embodiments, the method for preparing an engineered cell described herein includes genetically modifying a stem cell and differentiating the genetically modified stem cell into a T cell. In some embodiments, differentiating the genetically modified stem cell into a myeloid cell includes culturing the stem cell under conditions that induce T cell differentiation, leading to the generation of a T cell.

In some embodiments, the method for preparing an engineered cell described herein includes genetically modifying a stem cell and differentiating the genetically modified stem cell into an endocrine cell. In some embodiments, differentiating the genetically modified stem cell into a myeloid cell includes culturing the stem cell under conditions that induce endocrine cell differentiation, leading to the generation of an endocrine cell.

In some embodiments, the method for preparing an engineered cell described herein includes genetically modifying a stem cell and differentiating the genetically modified stem cell into an epithelial cell. In some embodiments, differentiating the genetically modified stem cell into a myeloid cell includes culturing the stem cell under conditions that induce epithelial cell differentiation, leading to the generation of an epithelial cell.

In some embodiments, the method for preparing an engineered cell described herein includes genetically modifying a stem cell and differentiating the genetically modified stem cell into a muscle cell. In some embodiments, differentiating the genetically modified stem cell into a myeloid cell includes culturing the stem cell under conditions that induce muscle cell differentiation, leading to the generation of a muscle cell.

In some embodiments, the stem cell is cultured in a bioreactor. In some embodiments, the stem cell is cultured in a cell factory under active gassing. By “active gassing” is meant exerting or applying a gas mixture pressure gradient in the cell factory or cell factories.

In some embodiments, the stem cell is differentiated into the myeloid cell using a multi-step process is used in which the stem cell is cultured with different combinations of cytokines and tissue culture media at each stage. For instance, in some embodiments, the differentiating the stem cell into a myeloid cell includes performing one or more of the following steps: First, contacting a cell culture with a first composition that includes BMP4 in a culture medium, when the cell culture is initially contacted with the first composition the cell culture includes the stem cell. A small molecule able to activate the same pathway as BMP4 can be used; Second, contacting the cell culture with a second composition that includes one or more of SCF, and VEGF, and optionally bFGF, (for example each of SCF, and VEGF, with or without bFGF) in a hematopoietic cell medium; Third, contacting the cell culture with a third composition that includes one or more of SCF, IL-3, TPO, M-CSF, and FLT3 ligand (for example each of SCF, IL-3, TPO, M-CSF, and FLT3 ligand) in a hematopoietic cell medium; and fourth, contacting the cell culture with a fourth composition that includes one or more of M-CSF, FLT3 ligand, and GM-CSF (for example each of M-CSF, FLT3 ligand, and GM-CSF) in a hematopoietic cell medium. In some embodiments all of the above four steps are performed in order. In some of such embodiments, the medium used for any of these four steps is a serum free medium. In some of such embodiments, the medium used for any of these four steps is a chemically defined medium. In the first of the above four steps, in some embodiments, a tissue culture medium suitable for maintenance of stem cells is used, while in other embodiments, a tissue culture medium suitable for differentiation of stem cells is used. In the last three of the above four steps, any suitable hematopoietic cell medium can be used.

In some embodiments, the method for preparing an engineered cell described herein further include expanding the engineered cell to produce a population of engineered cells. Expanding the engineered cell can include culturing or contacting the engineered cells with a medium having a cytokine and growth factor mixture permissive for expansion of the engineered cell.

In some embodiments, the method for preparing an engineered cell described herein further includes preserving the population of engineered cells after the expansion. For instance, the population of engineered cells can be cryopreserved. The cryopreserved population of engineered cells can be thawed at a later time and can be diluted for downstream applications.

In some embodiments, the population of engineered cells produced by the methods described herein is used in preparing a composition for treating a disease or disorder in a subject.

### III. Compositions

Certain aspects of the disclosure provide pharmaceutical compositions that include a population of engineered cells (e.g., a population of engineered cells described herein), and a pharmaceutical acceptable carrier, carrier, or diluent.

In some embodiments, the pharmaceutical composition includes a population of cells that includes at least one heterologous nucleic acid sequence encoding a polypeptide or a combination of polypeptides. In some embodiment, the polypeptide or combination of polypeptides is expressed at a level sufficient to inhibit natural killer cell mediated cytotoxicity.

In some embodiments, the polypeptide includes one or more of CLEC2D, TRAIL, SERPINB9, and HLA-C or a variant thereof. In some embodiments, the combination of polypeptides includes two or more of VISTA, CLEC2D, TRAIL, SERPINB9, HLA-C, and CD47 or a variant thereof. In some embodiments, the combination of polypeptides includes two or more of CLEC2D, TRAIL, SERPINB9, and HLA-C or a variant thereof. In some embodiments, the combination of polypeptides includes two or more of CLEC2D, TRAIL, and SERPINB9, or a variant thereof. In some embodiments, the combination of polypeptides includes three or more of VISTA, CLEC2D, TRAIL, SERPINB9, HLA-C, and CD47 or a variant thereof. In some embodiments, the combination of polypeptides includes three or more of CLEC2D, TRAIL, SERPINB9, and HLA-C or a variant thereof. In some embodiments, the combination of polypeptides includes CLEC2D, TRAIL, and SERPINB9, or a variant thereof. In some embodiments, the combination of polypeptides includes four or more of VISTA, CLEC2D, TRAIL, SERPINB9, HLA-C, and CD47 or a variant thereof. In some embodiments, the combination of polypeptides includes CLEC2D, TRAIL, SERPINB9, and HLA-C, or a variant thereof. In some embodiments, the combination of polypeptides includes five or more of VISTA, CLEC2D, TRAIL, SERPINB9, HLA-C, and CD47 or a variant thereof. In some embodiments, the combination of polypeptides includes VISTA, CLEC2D, TRAIL, SERPINB9, HLA-C, and CD47 or a variant thereof. In some embodiments, the combination of polypeptides includes one or more of VISTA, CLEC2D, TRAIL, and SERPINB9 or a variant thereof. In some embodiments, the combination of polypeptides includes CLEC2D or a variant thereof and TRAIL or a variant thereof. In some embodiments, the combination of polypeptides includes CLEC2D or a variant thereof and SERPINB9 or a variant thereof. In some embodiments, the polypeptide includes VISTA or a variant thereof. In some embodiments, the polypeptide includes human CLEC2D or a variant thereof. In some embodiments, the polypeptide includes human TRAIL or a variant thereof. In some embodiments, the polypeptide includes human SERPINB9 or a variant thereof. In some embodiments, the polypeptide includes human HLA-C or a variant thereof. In some embodiments, the polypeptide includes human CD47 or a variant thereof. In some embodiments, the pharmaceutical composition includes a population of engineered cells that includes at least two different heterologous nucleic acid sequences, each encoding a distinct polypeptide. In some embodiments, the pharmaceutical composition includes a population of engineered cells that includes at least three different heterologous nucleic acid sequences, each encoding a distinct polypeptide. In some embodiments, the pharmaceutical composition includes a population of engineered cells that includes at least four different heterologous nucleic acid sequences, each encoding a distinct polypeptide. In some embodiments, the pharmaceutical composition includes a population of engineered cells that includes at least five different heterologous nucleic acid sequences, each encoding a distinct polypeptide. In some embodiments, the pharmaceutical composition includes a population of engineered cells that includes at least six different heterologous nucleic acid sequences, each encoding a distinct polypeptide.

In some embodiments the pharmaceutical composition includes a population of engineered cells that includes at least two different heterologous nucleic acid sequences, each encoding CLEC2D or a variant thereof and SERPINB9 or a variant thereof. In some embodiments, the pharmaceutical composition includes a population of engineered cells that includes at least two different heterologous nucleic acid sequences, each encoding CLEC2D or a variant thereof and TRAIL or a variant thereof. In some embodiments, the pharmaceutical composition includes a population of engineered cells that includes at least two different heterologous nucleic acid sequences, each encoding CLEC2D or a variant thereof and HLA-C or a variant thereof. In some embodiments, the pharmaceutical composition includes a population of engineered cells that includes at least two different heterologous nucleic acid sequences, each encoding TRAIL or a variant thereof and SERPINB9 or a variant thereof. In some embodiments, the pharmaceutical composition includes a population of engineered cells that includes at least two different heterologous nucleic acid sequences, each encoding TRAIL or a variant thereof and HLA-C or a variant thereof. In some embodiments, the pharmaceutical composition includes a population of engineered cells that includes at least two different heterologous nucleic acid sequences, each encoding SERPINB9 or a variant thereof and HLA-C or a variant thereof.

In some embodiments, the polypeptide, including a variant of a polypeptide described herein, includes an amino acid sequence that has at least 65%, at least 70%, at least 75%, at least 80%, at least 85%, at least 90%, at least 95%, at least 96%, at least 97%, at least 98%, or at least 99% identity to any one of SEQ ID NOS: 1-11. In some embodiments, the polypeptide includes an amino acid sequence that has at least 65% identity to any one of SEQ ID NOS: 1-11. In some embodiments, the polypeptide includes an amino acid sequence that has at least 70% identity to any one of SEQ ID NOS: 1-11. In some embodiments, the polypeptide includes an amino acid sequence that has at least 75% identity to any one of SEQ ID NOS: 1-11. In some embodiments, the polypeptide includes an amino acid sequence that has at least 80% identity to any one of SEQ ID NOS: 1-11. In some embodiments, the polypeptide includes an amino acid sequence that has at least 85% identity to any one of SEQ ID NOS: 1-11. In some embodiments, the polypeptide includes an amino acid sequence that has at least 90% identity to any one of SEQ ID NOS: 1-11. In some embodiments, the polypeptide includes an amino acid sequence that has at least 95% identity to any one of SEQ ID NOS: 1-11. In some embodiments, the polypeptide includes an amino acid sequence that has at least 98% identity to any one of SEQ ID NOS: 1-11. In some embodiments, the polypeptide includes an amino acid sequence that has at least 99% identity to any one of SEQ ID NOS: 1-11. In some embodiments, the polypeptide includes an amino acid sequence that is an identical sequence to any one of SEQ ID NOS: 1-11.

In some embodiments, the polypeptide, including a variant of a polypeptide described herein, includes an amino acid sequence that has at least 65%, at least 70%, at least 75%, at least 80%, at least 85%, at least 90%, at least 95%, at least 96%, at least 97%, at least 98%, or at least 99% identity to any one of SEQ ID NOS: 2, 4, 5, and 11. In some embodiments, the polypeptide includes an amino acid sequence that has at least 65% identity to any one of SEQ ID NOS: 2, 4, 5, and 11. In some embodiments, the polypeptide includes an amino acid sequence that has at least 70% identity to any one of SEQ ID NOS: 2, 4, 5, and 11. In some embodiments, the polypeptide includes an amino acid sequence that has at least 75% identity to any one of SEQ ID NOS: 2, 4, 5, and 11. In some embodiments, the polypeptide includes an amino acid sequence that has at least 80% identity to any one of SEQ ID NOS: 2, 4, 5, and 11. In some embodiments, the polypeptide includes an amino acid sequence that has at least 85% identity to any one of SEQ ID NOS: 2, 4, 5, and 11. In some embodiments, the polypeptide includes an amino acid sequence that has at least 90% identity to any one of SEQ ID NOS: 2, 4, 5, and 11. In some embodiments, the polypeptide includes an amino acid sequence that has at least 95% identity to any one of SEQ ID NOS: 2, 4, 5, and 11. In some embodiments, the polypeptide includes an amino acid sequence that has at least 98% identity to any one of SEQ ID NOS: 2, 4, 5, and 11. In some embodiments, the polypeptide includes an amino acid sequence that has at least 99% identity to any one of SEQ ID NOS: 2, 4, 5, and 11. In some embodiments, the polypeptide includes an amino acid sequence that is an identical sequence to any one of SEQ ID NOS: 2, 4, 5, and 11.

In some embodiments, the polypeptide, including a variant of a polypeptide described herein, includes immune evasion activity. For instance, in some embodiments, the polypeptide or a variant of the polypeptide, when expressed by a cell at a suitable level, results in immune evasion activity. In some embodiments, the immune evasion activity includes inhibition of natural killer cell mediated cytotoxicity by the engineered cell. In some embodiments, the immune evasion activity include reduced T cell mediated killing of the engineered cell. In some embodiments, a polypeptide having at least 65%, at least 70%, at least 75%, at least 80%, at least 85%, at least 90%, at least 95%, at least 96%, at least 97%, at least 98%, or at least 99% identity to any one of SEQ ID NOS: 2, 4, 5, and 11, includes the immune evasion activity.

In some embodiments, the pharmaceutical composition includes a population of engineered cells that includes a polypeptide or a combination of polypeptides, or a variant of the polypeptide(s) that includes a biological activity of said polypeptide(s). For instance, in some embodiments, the population of engineered cells includes at least one heterologous nucleic acid sequence encoding any one of VISTA, CLEC2D, TRAIL, SERPINB9, HLA-C, and CD47 includes the biological activity associated with VISTA, CLEC2D, TRAIL, SERPINB9, HLA-C, and CD47, respectively. In some embodiments, a variant of VISTA, CLEC2D, TRAIL, SERPINB9, HLA-C, or CD47 includes the biological activity of VISTA, CLEC2D, TRAIL, SERPINB9, HLA-C, or CD47, respectively. In some embodiments, a polypeptide having at least 65%, at least 70%, at least 75%, at least 80%, at least 85%, at least 90%, at least 95%, at least 96%, at least 97%, at least 98%, or at least 99% identity to any one of SEQ ID NOS: 1-11, includes the biological activity associated with a polypeptide having an identical sequence to any one of SEQ ID NOS: 1-11, respectfully. In some embodiments, a polypeptide having at least 65%, at least 70%, at least 75%, at least 80%, at least 85%, at least 90%, at least 95%, at least 96%, at least 97%, at least 98%, or at least 99% identity to any one of SEQ ID NOS: 2, 4, 5, and 11, includes the biological activity associated with a polypeptide having an identical sequence to any one of SEQ ID NOS: 2, 4, 5, and 11, respectfully.

In some embodiments, the pharmaceutical composition includes a population of engineered cells that includes at least one heterologous nucleic acid sequence has at least 65%, at least 70%, at least 75%, at least 80%, at least 85%, at least 90%, at least 95%, at least 96%, at least 97%, at least 98%, or at least 99% identity to any one of SEQ ID NOS: 12-22. In some embodiments, the at least one heterologous nucleic acid sequence has at least 65% identity to any one of SEQ ID NOS: 12-22. In some embodiments, the at least one heterologous nucleic acid sequence has at least 75% identity to any one of SEQ ID NOS: 12-22. In some embodiments, the at least one heterologous nucleic acid sequence has at least 85% identity to any one of SEQ ID NOS: 12-22. In some embodiments, the at least one heterologous nucleic acid sequence has at least 90% identity to any one of SEQ ID NOS: 12-22. In some embodiments, the at least one heterologous nucleic acid sequence has at least 95% identity to any one of SEQ ID NOS: 12-22. In some embodiments, the at least one heterologous nucleic acid sequence has at least 98% identity to any one of SEQ ID NOS: 12-22. In some embodiments, the at least one heterologous nucleic acid sequence has at least 99% identity to any one of SEQ ID NOS: 12-22. In some embodiments, the at least one heterologous nucleic acid sequence has an identical sequence to one of SEQ ID NOS: 12-22.

In some embodiments, the pharmaceutical composition includes a population of engineered cells that includes at least one heterologous nucleic acid sequence has at least 65%, at least 70%, at least 75%, at least 80%, at least 85%, at least 90%, at least 95%, at least 96%, at least 97%, at least 98%, or at least 99% identity to any one of SEQ ID NOS: 13, 15, 17 and 22. In some embodiments, the at least one heterologous nucleic acid sequence has at least 65% identity to any one of SEQ ID NOS: 13, 15, 17 and 22. In some embodiments, the at least one heterologous nucleic acid sequence has at least 75% identity to any one of SEQ ID NOS: 13, 15, 17 and 22. In some embodiments, the at least one heterologous nucleic acid sequence has at least 85% identity to any one of SEQ ID NOS: 13, 15, 17 and 22. In some embodiments, the at least one heterologous nucleic acid sequence has at least 90% identity to any one of SEQ ID NOS: 13, 15, 17 and 22. In some embodiments, the at least one heterologous nucleic acid sequence has at least 95% identity to any one of SEQ ID NOS: 13, 15, 17 and 22. In some embodiments, the at least one heterologous nucleic acid sequence has at least 98% identity to any one of SEQ ID NOS: 13, 15, 17 and 22. In some embodiments, the at least one heterologous nucleic acid sequence has at least 99% identity to any one of SEQ ID NOS: 13, 15, 17 and 22. In some embodiments, the at least one heterologous nucleic acid sequence has an identical sequence to one of SEQ ID NOS: 13, 15, 17 and 22.

In some embodiments, the pharmaceutical composition includes a population of engineered cells that includes at least one heterologous nucleic acid sequence integrated into the genome of the engineered cell. In some embodiments, the at least one heterologous nucleic acid sequence is integrated into a sustained transgene expression locus (STEL) in the genome of the engineered cell. In some embodiments, the STEL is a gene locus that encodes a protein involved in one or more of: ribonucleoprotein complex formation, focal adhesion, cell-substrate adherens junction, cell-substrate junction, cell anchoring, extracellular exosome, extracellular vesicle, intracellular organelle, anchoring junction, RNA binding, nucleic acid binding (e.g., rRNA or mRNA binding), and protein binding. In some embodiments, the STEL is a glyceraldehyde 3-phosphate dehydrogenase (GAPDH) gene. In some embodiments, the STEL is a ribosomal protein gene locus, such as an RPL or RPS gene locus. Examples of RPL genes are RPL10, RPL13, RPS18, RPL3, RPLPI, RPL13A, RPL15, RPL41, RPL11, RPL32, RPL18A, RPL19, RPL28, RPL29, RPL9, RPL8, RPL6, RPL 18, RPL7, RPL7A, RPL21, RPL37A, RPL12, RPL5, RPL34, RPL35A, RPL30, RPL24, RPL39, RPL37, RPL14, RPL27A, RPLP2, RPLP0, RPL23A, RPL26, RPL36, RPL35, RPL23, RPL4, and RPL22. Examples of RPS genes are RPS2, RPS 19, RPS 14, RPS3A, RPS 12, RPS3, RPS6, RPS23, RPS27A, RPS8, RPS4X, RPS7, RPS24, RPS27, RPS15A, RPS9, RPS28, RPS 13, RPSA, RPS5, RPS16, RPS25, RPS15, RPS20, and RPSH. In some embodiments, the STEL is a gene locus encoding a mitochondrial protein, such as MT-COl, MT-C02, MT-ND4, MT-ND1, and MT-ND2. In some embodiments, the STEL is a gene locus encoding an actin protein, such as ACTG1 and ACTB. In some embodiments, the STEL is a gene locus encoding a eukaryotic translation elongation factor, such as EEF1A1 and EEF2, or a eukaryotic translation initiation factor such as EIEI. In some embodiments, the STEL is a gene locus encoding a histone, such as H3F3A and H3F3B. In some embodiments, the STEL is a gene locus selected from FTL, FTH1, TPT1, IMSB10, GAPDH, PTMA, GNB2L1, NACA, YBX1, NPM1, FAU, UBA52, HSP90AB1, MYL6, SERF2, and SRP14. Compositions and methods for integrating the heterologous nucleic acid into a STEL are described in International Patent Application Publication Nos. WO 2021/072329 and WO 2024/145653, which are incorporated by reference.

In some embodiments, the heterologous nucleic acid is integrated in the genome of the engineered cell in a sustained transcriptionally active payload region (STAPLR). In some embodiments, the STAPLR is selected from the group consisting of: the intergenic region between the RPL34 gene and the OSTC gene; the intergenic region between the ACTB gene and the FSCN1 gene; the intergenic region between the AKIRIN1 gene and the NDUFS5 gene; the intergenic region between the PRDX1 gene and the AKR1A1 gene; the intergenic region between the PTGES3 gene and the NACA gene; the intergenic region between the MLF2 gene and the PTMS gene; the intergenic region between the RAB13 gene and the RPS27 gene; the intergenic region between the JTB gene and the RAB13 gene; the intergenic region between the AKR1A1 gene and the NASP gene; the intergenic region between the NDUFS5 gene and the MACF1 gene; the intergenic region between the SRSF9 gene and the DYNLL1 gene; the intergenic region between the MYL6B gene and the MYL6 gene; the intergenic region between the GPX1 gene and the RHOA gene; the intergenic region between the HNRNPA2B1 gene and the CBX3 gene; the intergenic region between the ROMO gene and the RBM39 gene; and the intergenic region between the PA2G4 gene and the RPL41 gene. In some embodiments, the STAPLR is the intergenic region between the PRDX1 gene and the AKR1A1 gene. In some embodiments, the heterologous nucleic acid is integrated at a location that is at least 100-5000 base pairs away from the nearest gene. Compositions and methods for integrating a heterologous nucleic acid into a STAPLR are described in International Patent Application Publication No. WO 2024/145653, which are incorporated by reference.

In some embodiments, the pharmaceutical composition includes a population of engineered cells further that includes a kill switch. In some embodiments, the kill switch is a gene that encodes a herpes simplex virus thymidine kinase (HSV-TK), an inducible caspase9 (also known as incasep 9, and iCasp9), CD20, or a mutant human thymidylate kinase (mTMPK). In some embodiments, the kill switch is under the control of an inducible promoter. In some embodiments, the kill switch is encoded by a heterologous nucleic acid. In some embodiments, the at least one heterologous nucleic acid encoding the kill switch integrated into a different locus than the at least one heterologous nucleic acid encoding the polypeptide. In some embodiments, the at least one heterologous nucleic acid encoding the kill switch integrated into the same locus as the at least one heterologous nucleic acid encoding the polypeptide.

In some embodiments, the pharmaceutical composition includes a population of engineered cells further that includes a genetic modification that results in reduced T cell mediated killing as compared to a wild-type cell. In some embodiments, the population of engineered cells further include a genetic modification that results in at least 10%, at least 20%, at least 30%, at least 40%, at least 50%, at least 60%, at least 70%, at least 80%, at least 90%, at least 95%, or at least 100% reduced T cell mediated killing as compared to a wild-type cell. In some embodiments, the population of engineered cells further include a genetic modification that results in at least 20% reduced T cell mediated killing as compared to a wild-type cell. In some embodiments, the population of engineered cells further include a genetic modification that results in at least 50% reduced T cell mediated killing as compared to a wild-type cell. In some embodiments, the population of engineered cells further include a genetic modification that results in at least 80% reduced T cell mediated killing as compared to a wild-type cell. In some embodiments, the genetic modification that reduces T cell mediated killing of the engineered cell as compared to a wild-type cell includes a knockout of β2 microglobulin (B2M). In some embodiments, the genetic modification that reduces T cell mediated killing of the engineered cell as compared to a wild-type cell includes a knockout of HLA-A, B, and C molecules.

In some embodiments, the pharmaceutical composition includes a population of engineered cells further including a genetic modification that results in reduced T cell mediated killing as compared to a wild-type cell. In some embodiments, the pharmaceutical composition includes a population of engineered cells further including a genetic modification that results in at least 10%, at least 20%, at least 30%, at least 40%, at least 50%, at least 60%, at least 70%, at least 80%, at least 90%, at least 95%, or at least 100% reduced T cell mediated killing as compared to a wild-type cell. In some embodiments, the pharmaceutical composition includes a population of engineered cells further including a genetic modification that results in at least 20% reduced T cell mediated killing as compared to a wild-type cell. In some embodiments, the pharmaceutical composition includes a population of engineered cells further including a genetic modification that results in at least 50% reduced T cell mediated killing as compared to a wild-type cell. In some embodiments, the pharmaceutical composition includes a population of engineered cells further including a genetic modification that results in at least 80% reduced T cell mediated killing as compared to a wild-type cell.

In some embodiments, the genetic modification that results in reduced T cell mediated killing of the engineered cell as compared to a wild-type cell includes a deletion, disruption, or attenuation of a β2 microglobulin (B2M) gene. In some embodiments, the genetic modification that reduces T cell mediated killing of the engineered cell as compared to a wild-type cell includes a deletion, disruption, or attenuation of one or more of an HLA-A gene, an HLA-B gene, and an HLA-C gene.

In some embodiments, the pharmaceutical composition includes a population of engineered cells further that includes a deletion, disruption or attenuation in a CIITA gene or the RFX5 gene. Accordingly, in some embodiments, the pharmaceutical composition includes a population of engineered cells that includes a genetic modification that results in reduced T cell mediated killing of the engineered cell as compared to a wild-type cell as described herein (e.g., a deletion, disruption, or attenuation of a B2M, HLA-A, HLA-B, and/or HLA-C) and a deletion, disruption, or attenuation of the CIITA gene. In some embodiments, the pharmaceutical composition includes a population of engineered cells that includes a genetic modification that results in reduced T cell mediated killing of the engineered cell as compared to a wild-type cell as described herein (e.g., a deletion, disruption, or attenuation of a B2M, HLA-A, HLA-B, and/or HLA-C) and a deletion, disruption, or attenuation of the RFX5 gene. In some embodiments, the pharmaceutical composition includes a population of engineered cells that includes a genetic modification that results in reduced T cell mediated killing of the engineered cell as compared to a wild-type cell as described herein (e.g., a deletion, disruption, or attenuation of a B2M, HLA-A, HLA-B, and/or HLA-C) and a deletion, disruption, or attenuation of the CIITA gene and the RFX5 gene.

In some embodiments, the pharmaceutical composition includes a population of engineered cells further that includes at least one selectable marker gene.

In some embodiments, the pharmaceutical composition includes a population of engineered cells that is less susceptible to natural killer cell mediated cytotoxicity as compared to a population of cells not engineered to have the at least one heterologous nucleic acid sequence. In some embodiments, the population of engineered cells is at least about 10%, at least about 20%, at least about 30%, at least about 40%, at least about 50%, at least about 60%, at least about 70%, at least about 80%, or at least about 90% less susceptible to natural killer cell mediated cytotoxicity as compared to a population of cells not engineered to have the at least one heterologous nucleic acid sequence. In some embodiments, the population of engineered cells is at least about 10% less susceptible to natural killer cell mediated cytotoxicity as compared to a population of cells not engineered to have the at least one heterologous nucleic acid sequence. In some embodiments, the population of engineered cells is at least about 20% less susceptible to natural killer cell mediated cytotoxicity as compared to a population of cells not engineered to have the at least one heterologous nucleic acid sequence. In some embodiments, the population of engineered cells is at least about 30% less susceptible to natural killer cell mediated cytotoxicity as compared to a population of cells not engineered to have the at least one heterologous nucleic acid sequence. In some embodiments, the population of engineered cells is at least about 50% less susceptible to natural killer cell mediated cytotoxicity as compared to a population of cells not engineered to have the at least one heterologous nucleic acid sequence.

In some embodiments, the pharmaceutical composition includes a population of engineered cells including an engineered stem cell. In some embodiments, the engineered stem cell includes a pluripotent stem cell. In some embodiments, the pluripotent stem cell includes an embryonic stem cell (ESC) or induced pluripotent stem cell (iPSC). In some embodiments, the engineered stem cell includes a multipotent stem cell.

In some embodiments, the pharmaceutical composition includes a population of engineered cells including a cardiac cell, a neural cell, a T cell, retinal cell, endocrine cell, epithelial cell, muscle cell, or a myeloid cell. In some embodiments, the cardiac cell is a cardiomyocyte, a cardiac fibroblast, a cardiac smooth muscle cell, an epicardium cells, a cardiac endothelial cell, a Purkinje fiber, or a pacemaker cell. In some embodiments, the neural cell is a neuron or a glial cell. In some embodiments, the T cell is a helper T cell (CD4+ T cell), a cytotoxic T cell (CD8+ T cell), a memory T cell, a regulatory T cell (T reg cell), an innate-like T cell (e.g., a natural killer T cell), a mucosal-associated invariant T cell, or a gamma delta T cell, or a modified T cell (e.g., a CAR-T cell). In some embodiments, the retinal cell is a photoreceptor, a retinal horizontal cell, a retinal bipolar cell, a retinal amacrine cell, or a retinal ganglion cell. In some embodiments, the endocrine cell is a hypothalamus endocrine cell a pituitary gland endocrine cell, a pineal gland endocrine cell, a thyroid gland endocrine cell (e.g., a follicular cell), a parathyroid gland endocrine cell, a thymus gland endocrine cell, an adrenal gland endocrine cell, a pancreatic endocrine cell (e.g., an alpha cell, a beta cell, a delta cell or an F cell), an ovarian endocrine cell (e.g., a granulosa cell), or a testicular endocrine cell (e.g., a Leydig cell). In some embodiments, the epithelial cell is a squamous epithelial cell, a cuboidal epithelial cell, a columnar epithelial cell, a pseudostratified epithelia cell, or a stratified epithelial cell. In some embodiments, the muscle cell is a skeletal muscle cell, a cardiac muscle cell (e.g., a cardiomyocyte), or a smooth muscle cell. In some embodiments, the myeloid cell is a monocyte, a microglia, a macrophage, a dendritic cell, a basophil, an eosinophil, an erythrocyte, a mast cell, a neutrophil, a T cell, or any precursor progenitor cell thereof.

In some embodiments, the pharmaceutical composition includes a population of human engineered cells.

In some embodiments, the pharmaceutical composition is formulated for administration to a subject. The pharmaceutical composition be formulated for any suitable means of administration, such as for administration by local injection into a tissue.

The pharmaceutically acceptable excipient, carrier, or diluent can be any excipient, carrier or diluent known in the art. For instance, the pharmaceutically acceptable excipient, carrier, or diluent can be a cell culture medium (e.g., one that optionally lacks any animal-derived component), sterilized water, physiological saline, general buffers (e.g., phosphoric acid, citric acid, other organic acids, etc.), stabilizers, salts, anti-oxidants, surfactants, suspensions, isotonic agents, and/or preservatives can be included in a pharmaceutical composition described herein. The specific excipient, carrier, or diluent will depend on the route of administration intended for the pharmaceutical composition. In some embodiments, the pharmaceutically acceptable excipient, carrier, or diluent is an excipient, carrier, or diluent suitable for treatment of a tumor in a subject. In some embodiments, the pharmaceutically acceptable excipient, carrier, or diluent is an excipient, carrier, or diluent suitable for administration of the pharmaceutical composition by injection to a subject.

The pharmaceutical compositions described herein can be used for the treatment of a disease or disorder in a subject. In some embodiments, the pharmaceutical composition is for use in treating Parkinson's disease in a subject. In some embodiments, the Parkinson's disease is early Parkinson's disease. In some embodiments, the Parkinson's disease is advanced Parkinson's disease. In some embodiments, the pharmaceutical composition is for use in treating heart failure. In some embodiments, the pharmaceutical composition is for use in treating neural inflammation.

In some embodiments, the pharmaceutical composition includes a population of engineered cells allogenic to a subject that is administered the pharmaceutical composition. In some embodiments, the pharmaceutical composition includes a population of engineered cells autologous to a subject that is administered the pharmaceutical composition.

### IV. Methods

Certain aspects of the disclosure provide methods for treating a disease or disorder in a subject, or for achieving immune evasion of cells.

### Method of Treating a Disease or Disorder in a Subject

In some embodiments, the method of treating a disease or disorder in a subject includes administering a population of engineered cells (e.g., a population of engineered cells described herein), or a pharmaceutical composition that includes the population of engineered cells.

In some embodiments, the method of treating a disease or disorder in a subject includes administering a population of cells that includes at least one heterologous nucleic acid sequence encoding a polypeptide. In some embodiment, the polypeptide is expressed at a level sufficient to inhibit natural killer cell mediated cytotoxicity.

In some embodiments, the method includes administering a population of cells that includes at least one heterologous nucleic acid sequence encoding a polypeptide that includes one or more of VISTA, CLEC2D, TRAIL, SERPINB9, HLA-C, and CD47, or a variant thereof.

In some embodiments, the polypeptide includes one or more of CLEC2D, TRAIL, SERPINB9, and HLA-C or a variant thereof. In some embodiments, the combination of polypeptides include two or more of VISTA, CLEC2D, TRAIL, SERPINB9, HLA-C, and CD47 or a variant thereof. In some embodiments, the combination of polypeptides include two or more of CLEC2D, TRAIL, SERPINB9, and HLA-C or a variant thereof. In some embodiments, the combination of polypeptides include two or more of CLEC2D, TRAIL, and SERPINB9, or a variant thereof. In some embodiments, the combination of polypeptides include three or more of VISTA, CLEC2D, TRAIL, SERPINB9, HLA-C, and CD47 or a variant thereof. In some embodiments, the combination of polypeptides include three or more of CLEC2D, TRAIL, SERPINB9, and HLA-C or a variant thereof. In some embodiments, the combination of polypeptides include CLEC2D, TRAIL, and SERPINB9, or a variant thereof. In some embodiments, the combination of polypeptides include four or more of VISTA, CLEC2D, TRAIL, SERPINB9, HLA-C, and CD47 or a variant thereof. In some embodiments, the combination of polypeptides include CLEC2D, TRAIL, SERPINB9, and HLA-C, or a variant thereof. In some embodiments, the combination of polypeptides include five or more of VISTA, CLEC2D, TRAIL, SERPINB9, HLA-C, and CD47 or a variant thereof. In some embodiments, the combination of polypeptides include VISTA, CLEC2D, TRAIL, SERPINB9, HLA-C, and CD47 or a variant thereof. In some embodiments, the combination of polypeptides include one or more of VISTA, CLEC2D, TRAIL, and SERPINB9 or a variant thereof. In some embodiments, the combination of polypeptides include CLEC2D or a variant thereof and TRAIL or a variant thereof. In some embodiments, the combination of polypeptides include CLEC2D or a variant thereof and SERPINB9 or a variant thereof. In some embodiments, the polypeptide includes VISTA or a variant thereof. In some embodiments, the polypeptide includes human CLEC2D or a variant thereof. In some embodiments, the polypeptide includes human TRAIL or a variant thereof. In some embodiments, the polypeptide includes human SERPINB9 or a variant thereof. In some embodiments, the polypeptide includes human HLA-C or a variant thereof. In some embodiments, the polypeptide includes human CD47 or a variant thereof.

In some embodiments, the method includes administering a population of cells that includes at least one heterologous nucleic acid sequence encoding a polypeptide that includes an amino acid sequence that has at least 65%, at least 70%, at least 75%, at least 80%, at least 85%, at least 90%, at least 95%, at least 96%, at least 97%, at least 98%, or at least 99% identity to any one of SEQ ID NOS: 1-11. In some embodiments, the polypeptide includes an amino acid sequence that has at least 65% identity to any one of SEQ ID NOS: 1-11. In some embodiments, the polypeptide includes an amino acid sequence that has at least 70% identity to any one of SEQ ID NOS: 1-11. In some embodiments, the polypeptide includes an amino acid sequence that has at least 75% identity to any one of SEQ ID NOS: 1-11. In some embodiments, the polypeptide includes an amino acid sequence that has at least 80% identity to any one of SEQ ID NOS: 1-11. In some embodiments, the polypeptide includes an amino acid sequence that has at least 85% identity to any one of SEQ ID NOS: 1-11. In some embodiments, the polypeptide includes an amino acid sequence that has at least 90% identity to any one of SEQ ID NOS: 1-11. In some embodiments, the polypeptide includes an amino acid sequence that has at least 95% identity to any one of SEQ ID NOS: 1-11. In some embodiments, the polypeptide includes an amino acid sequence that has at least 98% identity to any one of SEQ ID NOS: 1-11. In some embodiments, the polypeptide includes an amino acid sequence that has at least 99% identity to any one of SEQ ID NOS: 1-11. In some embodiments, the polypeptide includes an amino acid sequence that is an identical sequence to any one of SEQ ID NOS: 1-11.

In some embodiments, the method includes administering a population of cells that includes at least one heterologous nucleic acid sequence encoding a polypeptide that includes an amino acid sequence that has at least 65%, at least 70%, at least 75%, at least 80%, at least 85%, at least 90%, at least 95%, at least 96%, at least 97%, at least 98%, or at least 99% identity to any one of SEQ ID NOS: 2, 4, 5, and 11. In some embodiments, the polypeptide includes an amino acid sequence that has at least 65% identity to any one of SEQ ID NOS: 2, 4, 5, and 11. In some embodiments, the polypeptide includes an amino acid sequence that has at least 70% identity to any one of SEQ ID NOS: 2, 4, 5, and 11. In some embodiments, the polypeptide includes an amino acid sequence that has at least 75% identity to any one of SEQ ID NOS: 2, 4, 5, and 11. In some embodiments, the polypeptide includes an amino acid sequence that has at least 80% identity to any one of SEQ ID NOS: 2, 4, 5, and 11. In some embodiments, the polypeptide includes an amino acid sequence that has at least 85% identity to any one of SEQ ID NOS: 2, 4, 5, and 11. In some embodiments, the polypeptide includes an amino acid sequence that has at least 90% identity to any one of SEQ ID NOS: 2, 4, 5, and 11. In some embodiments, the polypeptide includes an amino acid sequence that has at least 95% identity to any one of SEQ ID NOS: 2, 4, 5, and 11. In some embodiments, the polypeptide includes an amino acid sequence that has at least 98% identity to any one of SEQ ID NOS: 2, 4, 5, and 11. In some embodiments, the polypeptide includes an amino acid sequence that has at least 99% identity to any one of SEQ ID NOS: 2, 4, 5, and 11. In some embodiments, the polypeptide includes an amino acid sequence that is an identical sequence to any one of SEQ ID NOS: 2, 4, 5, and 11.

In some embodiments, the method includes administering a population of cells that includes at least one heterologous nucleic acid sequence encoding a, a variant of a polypeptide described herein, that includes immune evasion activity. For instance, in some embodiments, the polypeptide or a variant of the polypeptide, when expressed by a cell at a suitable level, results in immune evasion activity. In some embodiments, the immune evasion activity includes inhibition of natural killer cell mediated cytotoxicity by the engineered cell. In some embodiments, the immune evasion activity include reduced T cell mediated killing of the engineered cell. In some embodiments, a polypeptide having at least 65%, at least 70%, at least 75%, at least 80%, at least 85%, at least 90%, at least 95%, at least 96%, at least 97%, at least 98%, or at least 99% identity to any one of SEQ ID NOS: 1-11, includes the immune evasion activity. In some embodiments, a polypeptide having at least 65%, at least 70%, at least 75%, at least 80%, at least 85%, at least 90%, at least 95%, at least 96%, at least 97%, at least 98%, or at least 99% identity to any one of SEQ ID NOS: 2, 4, 5, and 11, includes the immune evasion activity.

In some embodiments, the method includes administering a population of cells that includes at least one heterologous nucleic acid sequence encoding a polypeptide, or aa variant of the polypeptide, that includes a biological activity of said polypeptide. For instance, in some embodiments, VISTA, CLEC2D, TRAIL, SERPINB9, HLA-C, and CD47 includes the biological activity associated with VISTA, CLEC2D, TRAIL, SERPINB9, HLA-C, and CD47, respectively. In some embodiments, a variant of VISTA, CLEC2D, TRAIL, SERPINB9, HLA-C, or CD47 includes the biological activity of VISTA, CLEC2D, TRAIL, SERPINB9, HLA-C, or CD47, respectively. In some embodiments, a polypeptide having at least 65%, at least 70%, at least 75%, at least 80%, at least 85%, at least 90%, at least 95%, at least 96%, at least 97%, at least 98%, or at least 99% identity to any one of SEQ ID NOS: 1-11, includes the biological activity associated with a polypeptide having an identical sequence to any one of SEQ ID NOS: 1-11, respectfully. In some embodiments, a polypeptide having at least 65%, at least 70%, at least 75%, at least 80%, at least 85%, at least 90%, at least 95%, at least 96%, at least 97%, at least 98%, or at least 99% identity to any one of SEQ ID NOS: 2, 4, 5, and 11, includes the biological activity associated with a polypeptide having an identical sequence to any one of SEQ ID NOS: 2, 4, 5, and 11, respectfully.

In some embodiments, the method includes administering a population of engineered cells that includes at least two different heterologous nucleic acid sequences, each encoding a distinct polypeptide. In some embodiments, method includes administering a population of engineered cells that includes at least three different heterologous nucleic acid sequences, each encoding a distinct polypeptide. In some embodiments, method includes administering a population of engineered cells that includes at least four different heterologous nucleic acid sequences, each encoding a distinct polypeptide. In some embodiments, method includes administering a population of engineered cells that includes at least five different heterologous nucleic acid sequences, each encoding a distinct polypeptide. In some embodiments, method includes administering a population of engineered cells that includes at least six different heterologous nucleic acid sequences, each encoding a distinct polypeptide.

In some embodiments, the method includes administering a population of engineered cells that includes at least two different heterologous nucleic acid sequences, each encoding CLEC2D or a variant thereof and SERPINB9 or a variant thereof. In some embodiments, the method includes administering a population of engineered cells that includes at least two different heterologous nucleic acid sequences, each encoding CLEC2D or a variant thereof and TRAIL or a variant thereof. In some embodiments, the method includes administering a population of engineered cells that includes at least two different heterologous nucleic acid sequences, each encoding CLEC2D or a variant thereof and HLA-C or a variant thereof. In some embodiments, the method includes administering a population of engineered cells that includes at least two different heterologous nucleic acid sequences, each encoding TRAIL or a variant thereof and SERPINB9 or a variant thereof. In some embodiments, the method includes administering a population of engineered cells that includes at least two different heterologous nucleic acid sequences, each encoding TRAIL or a variant thereof and HLA-C or a variant thereof. In some embodiments, the method includes administering a population of engineered cells that includes at least two different heterologous nucleic acid sequences, each encoding SERPINB9 or a variant thereof and HLA-C or a variant thereof.

In some embodiments, method includes administering a population of engineered cells that includes at least one heterologous nucleic acid sequence has at least 65%, at least 70%, at least 75%, at least 80%, at least 85%, at least 90%, at least 95%, at least 96%, at least 97%, at least 98%, or at least 99% identity to any one of SEQ ID NOS: 12-22. In some embodiments, the at least one heterologous nucleic acid sequence has at least 65% identity to any one of SEQ ID NOS: 12-22. In some embodiments, the at least one heterologous nucleic acid sequence has at least 75% identity to any one of SEQ ID NOS: 12-22. In some embodiments, the at least one heterologous nucleic acid sequence has at least 85% identity to any one of SEQ ID NOS: 12-22. In some embodiments, the at least one heterologous nucleic acid sequence has at least 90% identity to any one of SEQ ID NOS: 12-22. In some embodiments, the at least one heterologous nucleic acid sequence has at least 95% identity to any one of SEQ ID NOS: 12-22. In some embodiments, the at least one heterologous nucleic acid sequence has at least 98% identity to any one of SEQ ID NOS: 12-22. In some embodiments, the at least one heterologous nucleic acid sequence has at least 99% identity to any one of SEQ ID NOS: 12-22. In some embodiments, the at least one heterologous nucleic acid sequence has an identical sequence to one of SEQ ID NOS: 12-22.

In some embodiments, method includes administering a population of engineered cells that includes at least one heterologous nucleic acid sequence has at least 65%, at least 70%, at least 75%, at least 80%, at least 85%, at least 90%, at least 95%, at least 96%, at least 97%, at least 98%, or at least 99% identity to any one of SEQ ID NOS: 13, 15, 17 and 22. In some embodiments, the at least one heterologous nucleic acid sequence has at least 65% identity to any one of SEQ ID NOS: 13, 15, 17 and 22. In some embodiments, the at least one heterologous nucleic acid sequence has at least 75% identity to any one of SEQ ID NOS: 13, 15, 17 and 22. In some embodiments, the at least one heterologous nucleic acid sequence has at least 85% identity to any one of SEQ ID NOS: 13, 15, 17 and 22. In some embodiments, the at least one heterologous nucleic acid sequence has at least 90% identity to any one of SEQ ID NOS: 13, 15, 17 and 22. In some embodiments, the at least one heterologous nucleic acid sequence has at least 95% identity to any one of SEQ ID NOS: 13, 15, 17 and 22. In some embodiments, the at least one heterologous nucleic acid sequence has at least 98% identity to any one of SEQ ID NOS: 13, 15, 17 and 22. In some embodiments, the at least one heterologous nucleic acid sequence has at least 99% identity to any one of SEQ ID NOS: 13, 15, 17 and 22. In some embodiments, the at least one heterologous nucleic acid sequence has an identical sequence to one of SEQ ID NOS: 13, 15, 17 and 22.

In some embodiments, method includes administering a population of engineered cells that includes at least one heterologous nucleic acid sequence integrated into the genome of the engineered cell. In some embodiments, the at least one heterologous nucleic acid sequence is integrated into a sustained transgene expression locus (STEL) in the genome of the engineered cell. In some embodiments, the STEL is a gene locus that encodes a protein involved in one or more of: ribonucleoprotein complex formation, focal adhesion, cell-substrate adherens junction, cell-substrate junction, cell anchoring, extracellular exosome, extracellular vesicle, intracellular organelle, anchoring junction, RNA binding, nucleic acid binding (e.g., rRNA or mRNA binding), and protein binding. In some embodiments, the STEL is a glyceraldehyde 3-phosphate dehydrogenase (GAPDH) gene. In some embodiments, the STEL is a ribosomal protein gene locus, such as an RPL or RPS gene locus. Examples of RPL genes are RPL10, RPL13, RPS18, RPL3, RPLPI, RPL13A, RPL15, RPL41, RPL11, RPL32, RPL18A, RPL19, RPL28, RPL29, RPL9, RPL8, RPL6, RPL 18, RPL7, RPL7A, RPL21, RPL37A, RPL12, RPL5, RPL34, RPL35A, RPL30, RPL24, RPL39, RPL37, RPL14, RPL27A, RPLP2, RPLP0, RPL23A, RPL26, RPL36, RPL35, RPL23, RPL4, and RPL22. Examples of RPS genes are RPS2, RPS 19, RPS 14, RPS3A, RPS 12, RPS3, RPS6, RPS23, RPS27A, RPS8, RPS4X, RPS7, RPS24, RPS27, RPS15A, RPS9, RPS28, RPS 13, RPSA, RPS5, RPS16, RPS25, RPS15, RPS20, and RPSH. In some embodiments, the STEL is a gene locus encoding a mitochondrial protein, such as MT-COl, MT-C02, MT-ND4, MT-ND1, and MT-ND2. In some embodiments, the STEL is a gene locus encoding an actin protein, such as ACTG1 and ACTB. In some embodiments, the STEL is a gene locus encoding a eukaryotic translation elongation factor, such as EEF1A1 and EEF2, or a eukaryotic translation initiation factor such as EIEI. In some embodiments, the STEL is a gene locus encoding a histone, such as H3F3A and H3F3B. In some embodiments, the STEL is a gene locus selected from FTL, FTH1, TPT1, IMSB10, GAPDH, PTMA, GNB2L1, NACA, YBX1, NPM1, FAU, UBA52, HSP90AB1, MYL6, SERF2, and SRP14. Compositions and methods for integrating the heterologous nucleic acid into a STEL are described in International Patent Application Publication Nos. WO 2021/072329 and WO 2024/145653, which are incorporated by reference.

In some embodiments, the heterologous nucleic acid is integrated in the genome of the engineered cell in a sustained transcriptionally active payload region (STAPLR). In some embodiments, the STAPLR is selected from the group consisting of: the intergenic region between the RPL34 gene and the OSTC gene; the intergenic region between the ACTB gene and the FSCN1 gene; the intergenic region between the AKIRIN1 gene and the NDUFS5 gene; the intergenic region between the PRDX1 gene and the AKR1A1 gene; the intergenic region between the PTGES3 gene and the NACA gene; the intergenic region between the MLF2 gene and the PTMS gene; the intergenic region between the RAB13 gene and the RPS27 gene; the intergenic region between the JTB gene and the RAB13 gene; the intergenic region between the AKR1A1 gene and the NASP gene; the intergenic region between the NDUFS5 gene and the MACF1 gene; the intergenic region between the SRSF9 gene and the DYNLL1 gene; the intergenic region between the MYL6B gene and the MYL6 gene; the intergenic region between the GPX1 gene and the RHOA gene; the intergenic region between the HNRNPA2B1 gene and the CBX3 gene; the intergenic region between the ROMO gene and the RBM39 gene; and the intergenic region between the PA2G4 gene and the RPL41 gene. In some embodiments, the STAPLR is the intergenic region between the PRDX1 gene and the AKR1A1 gene. In some embodiments, the heterologous nucleic acid is integrated at a location that is at least 100-5000 base pairs away from the nearest gene. Compositions and methods for integrating a heterologous nucleic acid into a STAPLR are described in International Patent Application Publication No. WO 2024/145653, which are incorporated by reference.

In some embodiments, the method includes administering a population of engineered cells further that includes a kill switch. In some embodiments, the kill switch is a gene that encodes a herpes simplex virus thymidine kinase (HSV-TK), an inducible caspase9 (also known as incasep 9, and iCasp9), CD20, or a mutant human thymidylate kinase (mTMPK). In some embodiments, the kill switch is under the control of an inducible promoter. In some embodiments, the kill switch is encoded by a heterologous nucleic acid. In some embodiments, the at least one heterologous nucleic acid encoding the kill switch integrated into a different locus than the at least one heterologous nucleic acid encoding the polypeptide. In some embodiments, the at least one heterologous nucleic acid encoding the kill switch integrated into the same locus as the at least one heterologous nucleic acid encoding the polypeptide.

In some embodiments, the method includes administering a population of engineered cells that further includes a genetic modification that results in reduced T cell mediated killing as compared to a wild-type cell. In some embodiments, the engineered cell further includes a genetic modification that results in at least 10%, at least 20%, at least 30%, at least 40%, at least 50%, at least 60%, at least 70%, at least 80%, at least 90%, at least 95%, or at least 100% reduced T cell mediated killing as compared to a wild-type cell. In some embodiments, the engineered cell further includes a genetic modification that results in at least 20% reduced T cell mediated killing as compared to a wild-type cell. In some embodiments, the engineered cell further includes a genetic modification that results in at least 50% reduced T cell mediated killing as compared to a wild-type cell. In some embodiments, the engineered cell further includes a genetic modification that results in at least 80% reduced T cell mediated killing as compared to a wild-type cell. In some embodiments, the genetic modification that results in reduced T cell mediated killing of the engineered cell as compared to a wild-type cell includes a deletion, disruption, or attenuation of a β2 microglobulin (B2M) gene. In some embodiments, the genetic modification that reduces T cell mediated killing of the engineered cell as compared to a wild-type cell includes a deletion, disruption, or attenuation of one or more of an HLA-A gene, an HLA-B gene, and an HLA-C gene.

In some embodiments, the method includes administering a population of engineered cells further that includes a deletion, disruption or attenuation in a CIITA gene or the RFX5 gene. Accordingly, in some embodiments, the method includes administering a population of engineered cells that includes a genetic modification that results in reduced T cell mediated killing of the engineered cell as compared to a wild-type cell as described herein (e.g., a deletion, disruption, or attenuation of a B2M, HLA-A, HLA-B, and/or HLA-C) and a deletion, disruption, or attenuation of the CIITA gene. In some embodiments, the method includes administering a population of engineered cells that includes a genetic modification that results in reduced T cell mediated killing of the engineered cell as compared to a wild-type cell as described herein (e.g., a deletion, disruption, or attenuation of a B2M, HLA-A, HLA-B, and/or HLA-C) and a deletion, disruption, or attenuation of the RFX5 gene. In some embodiments, the method includes administering a population of engineered cells that includes a genetic modification that results in reduced T cell mediated killing of the engineered cell as compared to a wild-type cell as described herein (e.g., a deletion, disruption, or attenuation of a B2M, HLA-A, HLA-B, and/or HLA-C) and a deletion, disruption, or attenuation of the CIITA gene and the RFX5 gene.

In some embodiments, the method includes administering a population of engineered cells further that includes at least one selectable marker gene.

In some embodiments, the method includes administering a population of engineered cells that is less susceptible to natural killer cell mediated cytotoxicity as compared to a population of cells not engineered to have the at least one heterologous nucleic acid sequence. In some embodiments, the population of engineered cells is at least about 10%, at least about 20%, at least about 30%, at least about 40%, at least about 50%, at least about 60%, at least about 70%, at least about 80%, or at least about 90% less susceptible to natural killer cell mediated cytotoxicity as compared to a population of cells not engineered to have the at least one heterologous nucleic acid sequence. In some embodiments, the population of engineered cells is at least about 10% less susceptible to natural killer cell mediated cytotoxicity as compared to a population of cells not engineered to have the at least one heterologous nucleic acid sequence. In some embodiments, the population of engineered cells is at least about 20% less susceptible to natural killer cell mediated cytotoxicity as compared to a population of cells not engineered to have the at least one heterologous nucleic acid sequence. In some embodiments, the population of engineered cells is at least about 30% less susceptible to natural killer cell mediated cytotoxicity as compared to a population of cells not engineered to have the at least one heterologous nucleic acid sequence. In some embodiments, the population of engineered cells is at least about 50% less susceptible to natural killer cell mediated cytotoxicity as compared to a population of cells not engineered to have the at least one heterologous nucleic acid sequence. In some embodiments, method includes administering a population of engineered cells that includes an engineered stem cell. In some embodiments, the engineered stem cell is a pluripotent stem cell. In some embodiments, the pluripotent stem cell is an embryonic stem cell (ESC) or induced pluripotent stem cell (iPSC). In some embodiments, the engineered stem cell is a multipotent stem cell.

In some embodiments, the method includes administering a population of engineered cells that are positive for cardiac troponin T (cTNT). In some embodiments, at least 50%, at least 55%, at least 60%, at least 65%, at least 70%, at least 75%, at least 80%, at least 85%, at least 90%, at least 95%, at least 98%, or at least 99% of the engineered cells in the population of engineered cells are positive for cTnT. In some embodiments, at least 70% of the engineered cells in the population of engineered cells are positive for cTnT. In some embodiments, at least 80% of the engineered cells in the population of engineered cells are positive for cTnT. In some embodiments, at least 90% of the engineered cells in the population of engineered cells are positive for cTnT. In some embodiments, at least 95% of the engineered cells in the population of engineered cells are positive for cTnT. In some embodiments, at least 98% of the engineered cells in the population of engineered cells are positive for cTnT. In some embodiments, at least 99% of the engineered cells in the population of engineered cells are positive for cTnT.

In some embodiments, the method includes administering a population of engineered cells that includes a cardiac cell, a neural cell, a T cell, a retinal cell, an endocrine cell, an epithelial cell, a muscle cell, or a myeloid cell. In some embodiments, the cardiac cell is a cardiomyocyte, a cardiac fibroblast, a cardiac smooth muscle cell, an epicardium cells, a cardiac endothelial cell, a Purkinje fiber, or a pacemaker cell. In some embodiments, the neural cell is a neuron or a glial cell. In some embodiments, the T cell is a helper T cell (CD4+ T cell), a cytotoxic T cell (CD8+ T cell), a memory T cell, a regulatory T cell (T reg cell), an innate-like T cell (e.g., a natural killer T cell), a mucosal-associated invariant T cell, or a gamma delta T cell, or a modified T cell (e.g., a CAR-T cell). In some embodiments, the retinal cell is a photoreceptor, a retinal horizontal cell, a retinal bipolar cell, a retinal amacrine cell, or a retinal ganglion cell. In some embodiments, the endocrine cell is a hypothalamus endocrine cell a pituitary gland endocrine cell, a pineal gland endocrine cell, a thyroid gland endocrine cell (e.g., a follicular cell), a parathyroid gland endocrine cell, a thymus gland endocrine cell, an adrenal gland endocrine cell, a pancreatic endocrine cell (e.g., an alpha cell, a beta cell, a delta cell or an F cell), an ovarian endocrine cell (e.g., a granulosa cell), or a testicular endocrine cell (e.g., a Leydig cell). In some embodiments, the epithelial cell is a squamous epithelial cell, a cuboidal epithelial cell, a columnar epithelial cell, a pseudostratified epithelia cell, or a stratified epithelial cell. In some embodiments, the muscle cell is a skeletal muscle cell, a cardiac muscle cell (e.g., a cardiomyocyte), or a smooth muscle cell. In some embodiments, the myeloid cell is a monocyte, a microglia, a macrophage, a dendritic cell, a basophil, an eosinophil, an erythrocyte, a mast cell, a neutrophil, or any precursor progenitor cell thereof. In some embodiments, the cardiac cell, the neural cell, the T cell, the retinal cell, the endocrine cell, the epithelial cell, the muscle cell, or the myeloid cell are derived from a stem cell.

In some embodiments, the method includes administering a population of human engineered cells to the subject. In some embodiments, the subject is a human. In some embodiments, the population of cells is allogenic to the subject. In some embodiments, the population of cells is autologous to the subject.

In some embodiments, the disease or disorder to be treated by the methods described herein is Parkinson's disease, multiple sclerosis, irritable bowel syndrome, type 1 diabetes, rheumatoid arthritis, heart failure, liver disease, cancer, inflammation, or neural inflammation. In some embodiments, the method is for treating Parkinson's disease in a subject. In some embodiments, the Parkinson's disease is early Parkinson's disease. In some embodiments, the Parkinson's disease is advanced Parkinson's disease. In some embodiments, the method if for treating heart failure in a subject. In some embodiments, the method if for treating neural inflammation in a subject.

In some embodiments, the administration of the population of engineered cells results in improvement of at least one symptom associated with the disease or disorder, e.g., as determined by responsiveness/non-responsiveness, or indicators known in the art. The choice of technologies and methods, and the time and frequency of examination can be determined and/or adjusted by a person skilled in the art based on the subject's specific condition.

In some embodiments, the method further includes examining the subject for responsiveness to the population of engineered cells. The responsiveness of the subject can be determined as an improvement of at least one parameter of disease progression. The subject can have partial response or complete response to a treatment. The response to a treatment can be determined based on methods known in the art. A person skilled in the art can determine the proper methods based on the type of diseases being evaluated.

In some embodiments, the method further includes administering a kill switch activator after administration of the population of engineered cells. The kill switch activator can be used for removal of the population of engineered cells after a period of time. The kill switch activator can be administered any time following administration of the population of engineered cells, such as for example, after the subject has achieved a certain level of responsiveness as measured by improvement of at least one symptom associated with the disease or disorder. In some embodiments, the method includes administering an activator of a kill switch selected from a gene that encodes a herpes simplex virus thymidine kinase (HSV-TK), an inducible caspase9 (also known as incasep 9, and iCasp9), CD20, or a mutant human thymidylate kinase (mTMPK).

The population of engineered cells of the methods described herein can be administered to a subject by any suitable route of administration. Methods of administration of populations of engineered cells described herein are known to those in the art. In some embodiments, the method includes administering the population of engineered cells orally, pulmonarily, intranasally, parenterally (intravenously, intramuscularly, intraperitoneally, or subcutaneously), rectally, intralymphatically, or topically. In some embodiments, the population of engineered cells is administered by directly injecting into a tissue. For instance, in some embodiments, the population of engineered cells is administered by direct injection into a subject's heart by, for example, intracoronary administration, intramyocardial administration, or transendocardial administration. The administration of the population of engineered cells to a subject can be performed by any suitable means, such as by injection, by catheter, by an implantable device, or by any other device suitable for administration. By way of example, the population of engineered cells can be introduced to the heart by using a catheter inserted via the femoral, subclavian, jugular or axillary vein, or by endocardial transplantation into the ventricle or atrium region. The population of engineered cells also can be transplanted into the ventricle or atrium region by an epicardial approach, using a needle inserted through the chest.

The population of engineered cells can be administered to the subject at one time or over a series of administrations and can be administered to the patient at any time from diagnosis of the disease or disorder onwards. For instance, the population of engineered cells can be administered as one or more doses over the course of a treatment. The doses can be administered using the same or a different route of administration, and can contain the same amount or a different amount of the population of engineered cells.

The duration of administration can depend on the route of administration of the population of engineered cells.

In some embodiments, the methods further includes administering an additional therapy to the subject. The additional therapy will depend on the disease or disorder being treated by the methods described herein.

### Methods of Achieving Immune Evasion

In some aspects of the disclosure, provided herein are methods of achieving immune evasion of a cell.

In some embodiments, the method for achieving immune evasion includes genetically modifying a cell to increase the expression of at least one polypeptide. In some embodiments, the polypeptide is expressed at a level sufficient to inhibit natural killer mediated cytotoxicity. In some embodiments, the genetically modified cell exhibits increased survival when challenged with natural killer cells as compared to a wild-type cell.

In some embodiments, the method includes genetically modifying a cell to increase expression of a polypeptide that includes one or more of VISTA, CLEC2D, TRAIL, SERPINB9, HLA-C, and CD47, or a variant thereof.

In some embodiments, the polypeptide includes one or more of CLEC2D, TRAIL, SERPINB9, and HLA-C or a variant thereof. In some embodiments, the combination of polypeptides include two or more of VISTA, CLEC2D, TRAIL, SERPINB9, HLA-C, and CD47 or a variant thereof. In some embodiments, the combination of polypeptides include two or more of CLEC2D, TRAIL, SERPINB9, and HLA-C or a variant thereof. In some embodiments, the combination of polypeptides include two or more of CLEC2D, TRAIL, and SERPINB9, or a variant thereof. In some embodiments, the combination of polypeptides include three or more of VISTA, CLEC2D, TRAIL, SERPINB9, HLA-C, and CD47 or a variant thereof. In some embodiments, the combination of polypeptides include three or more of CLEC2D, TRAIL, SERPINB9, and HLA-C or a variant thereof. In some embodiments, the combination of polypeptides include CLEC2D, TRAIL, and SERPINB9, or a variant thereof. In some embodiments, the combination of polypeptides include four or more of VISTA, CLEC2D, TRAIL, SERPINB9, HLA-C, and CD47 or a variant thereof. In some embodiments, the combination of polypeptides include CLEC2D, TRAIL, SERPINB9, and HLA-C, or a variant thereof. In some embodiments, the combination of polypeptides include five or more of VISTA, CLEC2D, TRAIL, SERPINB9, HLA-C, and CD47 or a variant thereof. In some embodiments, the combination of polypeptides include VISTA, CLEC2D, TRAIL, SERPINB9, HLA-C, and CD47 or a variant thereof. In some embodiments, the combination of polypeptides include one or more of VISTA, CLEC2D, TRAIL, and SERPINB9 or a variant thereof. In some embodiments, the combination of polypeptides include CLEC2D or a variant thereof and TRAIL or a variant thereof. In some embodiments, the combination of polypeptides include CLEC2D or a variant thereof and SERPINB9 or a variant thereof. In some embodiments, the polypeptide includes VISTA or a variant thereof. In some embodiments, the polypeptide includes human CLEC2D or a variant thereof. In some embodiments, the polypeptide includes human TRAIL or a variant thereof. In some embodiments, the polypeptide includes human SERPINB9 or a variant thereof. In some embodiments, the polypeptide includes human HLA-C or a variant thereof. In some embodiments, the polypeptide includes human CD47 or a variant thereof.

In some embodiments, the method includes genetically modifying a cell to increase expression of a polypeptide that includes an amino acid sequence that has at least 65%, at least 70%, at least 75%, at least 80%, at least 85%, at least 90%, at least 95%, at least 96%, at least 97%, at least 98%, or at least 99% identity to any one of SEQ ID NOS: 1-11. In some embodiments, the polypeptide includes an amino acid sequence that has at least 65% identity to any one of SEQ ID NOS: 1-11. In some embodiments, the polypeptide includes an amino acid sequence that has at least 70% identity to any one of SEQ ID NOS: 1-11. In some embodiments, the polypeptide includes an amino acid sequence that has at least 75% identity to any one of SEQ ID NOS: 1-11. In some embodiments, the polypeptide includes an amino acid sequence that has at least 80% identity to any one of SEQ ID NOS: 1-11. In some embodiments, the polypeptide includes an amino acid sequence that has at least 85% identity to any one of SEQ ID NOS: 1-11. In some embodiments, the polypeptide includes an amino acid sequence that has at least 90% identity to any one of SEQ ID NOS: 1-11. In some embodiments, the polypeptide includes an amino acid sequence that has at least 95% identity to any one of SEQ ID NOS: 1-11. In some embodiments, the polypeptide includes an amino acid sequence that has at least 98% identity to any one of SEQ ID NOS: 1-11. In some embodiments, the polypeptide includes an amino acid sequence that has at least 99% identity to any one of SEQ ID NOS: 1-11. In some embodiments, the polypeptide includes an amino acid sequence that is an identical sequence to any one of SEQ ID NOS: 1-11.

In some embodiments, the method includes genetically modifying a cell to increase expression of a polypeptide that includes an amino acid sequence that has at least 65%, at least 70%, at least 75%, at least 80%, at least 85%, at least 90%, at least 95%, at least 96%, at least 97%, at least 98%, or at least 99% identity to any one of SEQ ID NOS: 2, 4, 5, and 11. In some embodiments, the polypeptide includes an amino acid sequence that has at least 65% identity to any one of SEQ ID NOS: 2, 4, 5, and 11. In some embodiments, the polypeptide includes an amino acid sequence that has at least 70% identity to any one of SEQ ID NOS: 2, 4, 5, and 11. In some embodiments, the polypeptide includes an amino acid sequence that has at least 75% identity to any one of SEQ ID NOS: 2, 4, 5, and 11. In some embodiments, the polypeptide includes an amino acid sequence that has at least 80% identity to any one of SEQ ID NOS: 2, 4, 5, and 11. In some embodiments, the polypeptide includes an amino acid sequence that has at least 85% identity to any one of SEQ ID NOS: 2, 4, 5, and 11. In some embodiments, the polypeptide includes an amino acid sequence that has at least 90% identity to any one of SEQ ID NOS: 2, 4, 5, and 11. In some embodiments, the polypeptide includes an amino acid sequence that has at least 95% identity to any one of SEQ ID NOS: 2, 4, 5, and 11. In some embodiments, the polypeptide includes an amino acid sequence that has at least 98% identity to any one of SEQ ID NOS: 2, 4, 5, and 11. In some embodiments, the polypeptide includes an amino acid sequence that has at least 99% identity to any one of SEQ ID NOS: 2, 4, 5, and 11. In some embodiments, the polypeptide includes an amino acid sequence that is an identical sequence to any one of SEQ ID NOS: 2, 4, 5, and 11.

In some embodiments, the polypeptide, including a variant of a polypeptide described herein, includes immune evasion activity. For instance, in some embodiments, the polypeptide or a variant of the polypeptide, when expressed by a cell at a suitable level, results in immune evasion activity. In some embodiments, the immune evasion activity includes inhibition of natural killer cell mediated cytotoxicity by the engineered cell. In some embodiments, the immune evasion activity include reduced T cell mediated killing of the engineered cell. In some embodiments, a polypeptide having at least 65%, at least 70%, at least 75%, at least 80%, at least 85%, at least 90%, at least 95%, at least 96%, at least 97%, at least 98%, or at least 99% identity to any one of SEQ ID NOS: 1-11, includes the immune evasion activity. In some embodiments, a polypeptide having at least 65%, at least 70%, at least 75%, at least 80%, at least 85%, at least 90%, at least 95%, at least 96%, at least 97%, at least 98%, or at least 99% identity to any one of SEQ ID NOS: 2, 4, 5, and 11, includes the immune evasion activity.

In some embodiments, the polypeptide or a variant of the polypeptide includes a biological activity of said polypeptide. For instance, in some embodiments, VISTA, CLEC2D, TRAIL, SERPINB9, HLA-C, and CD47 includes the biological activity associated with VISTA, CLEC2D, TRAIL, SERPINB9, HLA-C, and CD47, respectively. In some embodiments, a variant of VISTA, CLEC2D, TRAIL, SERPINB9, HLA-C, or CD47 includes the biological activity of VISTA, CLEC2D, TRAIL, SERPINB9, HLA-C, or CD47, respectively. In some embodiments, a polypeptide having at least 65%, at least 70%, at least 75%, at least 80%, at least 85%, at least 90%, at least 95%, at least 96%, at least 97%, at least 98%, or at least 99% identity to any one of SEQ ID NOS: 1-11, includes the biological activity associated with a polypeptide having an identical sequence to any one of SEQ ID NOS: 1-11, respectfully. In some embodiments, a polypeptide having at least 65%, at least 70%, at least 75%, at least 80%, at least 85%, at least 90%, at least 95%, at least 96%, at least 97%, at least 98%, or at least 99% identity to any one of SEQ ID NOS: 2, 4, 5, and 11, includes the biological activity associated with a polypeptide having an identical sequence to any one of SEQ ID NOS: 2, 4, 5, and 11, respectfully.

In some embodiments, genetically modifying the cell includes integrating at least one heterologous nucleic acid sequence encoding the polypeptide into the genome of the cell. In some embodiments, genetically modifying the cell includes integrating at least one heterologous nucleic acid sequence encoding the polypeptide has at least 65%, at least 70%, at least 75%, at least 80%, at least 85%, at least 90%, at least 95%, at least 96%, at least 97%, at least 98%, or at least 99% identity to any one of SEQ ID NOS: 1-11, into the genome of the cell. In some embodiments, the at least one heterologous nucleic acid sequence has at least 65% identity to any one of SEQ ID NOS: 1-11. In some embodiments, the at least one heterologous nucleic acid sequence has at least 75% identity to any one of SEQ ID NOS: 1-11. In some embodiments, the at least one heterologous nucleic acid sequence has at least 85% identity to any one of SEQ ID NOS: 1-11. In some embodiments, the at least one heterologous nucleic acid sequence has at least 90% identity to any one of SEQ ID NOS: 1-11. In some embodiments, the at least one heterologous nucleic acid sequence has at least 95% identity to any one of SEQ ID NOS: 1-11. In some embodiments, the at least one heterologous nucleic acid sequence has at least 98% identity to any one of SEQ ID NOS: 1-11. In some embodiments, the at least one heterologous nucleic acid sequence has at least 99% identity to any one of SEQ ID NOS: 1-11. In some embodiments, the at least one heterologous nucleic acid sequence has an identical sequence to one of SEQ ID NOS: 1-11.

In some embodiments, genetically modifying the cell includes integrating at least one heterologous nucleic acid sequence encoding the polypeptide into the genome of the cell. In some embodiments, genetically modifying the cell includes integrating at least one heterologous nucleic acid sequence encoding the polypeptide has at least 65%, at least 70%, at least 75%, at least 80%, at least 85%, at least 90%, at least 95%, at least 96%, at least 97%, at least 98%, or at least 99% identity to any one of SEQ ID NOS: 13, 15, 17 and 22, into the genome of the cell. In some embodiments, the at least one heterologous nucleic acid sequence has at least 65% identity to any one of SEQ ID NOS: 13, 15, 17 and 22. In some embodiments, the at least one heterologous nucleic acid sequence has at least 75% identity to any one of SEQ ID NOS: 13, 15, 17 and 22. In some embodiments, the at least one heterologous nucleic acid sequence has at least 85% identity to any one of SEQ ID NOS: 13, 15, 17 and 22. In some embodiments, the at least one heterologous nucleic acid sequence has at least 90% identity to any one of SEQ ID NOS: 13, 15, 17 and 22. In some embodiments, the at least one heterologous nucleic acid sequence has at least 95% identity to any one of SEQ ID NOS: 13, 15, 17 and 22. In some embodiments, the at least one heterologous nucleic acid sequence has at least 98% identity to any one of SEQ ID NOS: 13, 15, 17 and 22. In some embodiments, the at least one heterologous nucleic acid sequence has at least 99% identity to any one of SEQ ID NOS: 13, 15, 17 and 22. In some embodiments, the at least one heterologous nucleic acid sequence has an identical sequence to one of SEQ ID NOS: 13, 15, 17 and 22.

In some embodiments, the at least one heterologous nucleic acid sequence encoding the polypeptide is integrated into the genome of the cell. The CRISPR/Cas system has been used for introducing genetic modifications and gene regulation in various species. Without being limited by theory, a target nucleic acid can be modified by the interaction of the CRISPR/Cas system and a sequence present in the target nucleic acid, for example, to cause cleavage (e.g., hydrolysis of one or more phosphodiester bonds) of the target nucleic acid and introduce the genetic modification. In some embodiments, the at least one heterologous nucleic acid sequence encoding the polypeptide is integrated into the genome of the cell using a CRISPR/Cas9 system or a CRISPR/Cas12 system.

In some embodiments, the method for achieving immune evasion includes integrating the at least one heterologous nucleic acid sequence encoding the polypeptide into a STEL in the genome of the cell. In some embodiments, the STEL is a gene locus that encodes a protein involved in one or more of: ribonucleoprotein complex formation, focal adhesion, cell-substrate adherens junction, cell-substrate junction, cell anchoring, extracellular exosome, extracellular vesicle, intracellular organelle, anchoring junction, RNA binding, nucleic acid binding (e.g., rRNA or mRNA binding), and protein binding. In some embodiments, the STEL is a glyceraldehyde 3-phosphate dehydrogenase (GAPDH) gene. In some embodiments, the STEL is a ribosomal protein gene locus, such as an RPL or RPS gene locus. Examples of RPL genes are RPL10, RPL13, RPS18, RPL3, RPLPI, RPL13A, RPL15, RPL41, RPL11, RPL32, RPL18A, RPL19, RPL28, RPL29, RPL9, RPL8, RPL6, RPL 18, RPL7, RPL7A, RPL21, RPL37A, RPL12, RPL5, RPL34, RPL35A, RPL30, RPL24, RPL39, RPL37, RPL14, RPL27A, RPLP2, RPLP0, RPL23A, RPL26, RPL36, RPL35, RPL23, RPL4, and RPL22. Examples of RPS genes are RPS2, RPS 19, RPS 14, RPS3A, RPS 12, RPS3, RPS6, RPS23, RPS27A, RPS8, RPS4X, RPS7, RPS24, RPS27, RPS15A, RPS9, RPS28, RPS 13, RPSA, RPS5, RPS16, RPS25, RPS15, RPS20, and RPSII. In some embodiments, the STEL is a gene locus encoding a mitochondrial protein, such as MT-COl, MT-C02, MT-ND4, MT-ND1, and MT-ND2. In some embodiments, the STEL is a gene locus encoding an actin protein, such as ACTG1 and ACTB. In some embodiments, the STEL is a gene locus encoding a eukaryotic translation elongation factor, such as EEF1A1 and EEF2, or a eukaryotic translation initiation factor such as EIEI. In some embodiments, the STEL is a gene locus encoding a histone, such as H3F3A and H3F3B. In some embodiments, the STEL is a gene locus selected from FTL, FTH1, TPT1, IMSB10, GAPDH, PTMA, GNB2L1, NACA, YBX1, NPM1, FAU, UBA52, HSP90AB1, MYL6, SERF2, and SRP14. Compositions and methods for integrating the heterologous nucleic acid into a STEL are described in International Patent Application Publication Nos. WO 2021/072329 and WO 2024/145653, which are incorporated by reference.

In some embodiments, the heterologous nucleic acid is integrated in the genome of the engineered cell in a sustained transcriptionally active payload region (STAPLR). In some embodiments, the STAPLR is selected from the group consisting of: the intergenic region between the RPL34 gene and the OSTC gene; the intergenic region between the ACTB gene and the FSCN1 gene; the intergenic region between the AKIRIN1 gene and the NDUFS5 gene; the intergenic region between the PRDX1 gene and the AKR1A1 gene; the intergenic region between the PTGES3 gene and the NACA gene; the intergenic region between the MLF2 gene and the PTMS gene; the intergenic region between the RAB13 gene and the RPS27 gene; the intergenic region between the JTB gene and the RAB13 gene; the intergenic region between the AKR1A1 gene and the NASP gene; the intergenic region between the NDUFS5 gene and the MACF1 gene; the intergenic region between the SRSF9 gene and the DYNLL1 gene; the intergenic region between the MYL6B gene and the MYL6 gene; the intergenic region between the GPX1 gene and the RHOA gene; the intergenic region between the HNRNPA2B1 gene and the CBX3 gene; the intergenic region between the ROMO gene and the RBM39 gene; and the intergenic region between the PA2G4 gene and the RPL41 gene. In some embodiments, the STAPLR is the intergenic region between the PRDX1 gene and the AKR1A1 gene. In some embodiments, the heterologous nucleic acid is integrated at a location that is at least 100-5000 base pairs away from the nearest gene. Compositions and methods for integrating a heterologous nucleic acid into a STAPLR are described in International Patent Application Publication No. WO 2024/145653, which are incorporated by reference.

In some embodiments, the at least one heterologous nucleic acid sequence encoding the polypeptide further includes one or more regulatory elements. In some embodiments, the regulatory element is a promoter. In some embodiments, expression of the polypeptide is driven by an endogenous gene promoter.

In some embodiments, at least one heterologous nucleic acid sequence encoding the polypeptide is introduced into the cell in a vector. The vector can be plasmid, virus, or other nucleic acid designed for introducing a nucleic acid of interest into a cell. The vector is used to introduce a gene of interest into a host cell in which the vector will interact with polymerases in the cell to express the protein encoded in the vector. The vector can exist in the cell extra-chromosomally or integrated into the genome of the host cell.

In some embodiments, the method for achieving immune evasion further includes introducing at least one heterologous nucleic acid encoding a kill switch into the cell. In some embodiments, the kill switch is a gene that encodes a herpes simplex virus thymidine kinase (HSV-TK), an inducible caspase9 (also known as incasep 9, and iCasp9), CD20, or a mutant human thymidylate kinase (mTMPK). In some embodiments, the kill switch is under the control of an inducible promoter. some embodiments, the at least one heterologous nucleic acid encoding the kill switch is integrated into the genome of the cell using the CRISPR/Cas system. The at least one heterologous nucleic acid encoding the kill switch can be integrated into the same or a different locus as the at least one heterologous nucleic acid sequence encoding the polypeptide.

In some embodiments, the at least one heterologous nucleic acid encoding the kill switch is introduced into the cell in a vector. The at least one heterologous nucleic acid encoding the kill switch can be introduced into the cell in the same vector or a different vector as the at least one heterologous nucleic acid sequence encoding the polypeptide.

In some embodiments, the method for achieving immune evasion further includes introducing a genetic modification to the cell that results in reduced T cell mediated killing of the genetically modified cell as compared to a wild-type cell. In some embodiments, the genetic modification results in at least 20% reduced T cell mediated killing of the genetically modified cell as compared to a wild-type cell. In some embodiments, the genetic modification results in at least 50% reduced T cell mediated killing of the genetically modified cell as compared to a wild-type cell. In some embodiments, the e genetic modification results in at least 80% reduced T cell mediated killing of the genetically modified cell as compared to a wild-type cell.

In some embodiments, the genetic modification that results in reduced T cell mediated killing of the engineered cell as compared to a wild-type cell includes a deletion, disruption, or attenuation of a β2 microglobulin (B2M) gene. In some embodiments, the genetic modification that reduces T cell mediated killing of the engineered cell as compared to a wild-type cell includes a deletion, disruption, or attenuation of one or more of an HLA-A gene, an HLA-B gene, and an HLA-C gene.

In some embodiments, the method for achieving immune evasion further includes introducing a deletion, disruption or attenuation in a CIITA gene and the RFX5 gene. Accordingly, in some embodiments, the method for achieving immune evasion includes introducing a genetic modification that results in reduced T cell mediated killing of the engineered cell as compared to a wild-type cell as described herein (e.g., a deletion, disruption, or attenuation of a B2M, HLA-A, HLA-B, and/or HLA-C) and a deletion, disruption, or attenuation of the CIITA gene. In some embodiments, the method for achieving immune evasion includes introducing a genetic modification that results in reduced T cell mediated killing of the engineered cell as compared to a wild-type cell as described herein (e.g., a deletion, disruption, or attenuation of a B2M, HLA-A, HLA-B, and/or HLA-C) and a deletion, disruption, or attenuation of the RFX5 gene. In some embodiments, the method for achieving immune evasion includes introducing a genetic modification that results in reduced T cell mediated killing of the engineered cell as compared to a wild-type cell as described herein (e.g., a deletion, disruption, or attenuation of a B2M, HLA-A, HLA-B, and/or HLA-C) and a deletion, disruption, or attenuation of the CIITA gene and the RFX5 gene.

In some embodiments, the method for achieving immune evasion includes genetically modifying a stem cell. In some embodiments, the stem cell is a pluripotent stem cell. In some embodiments, the pluripotent stem cell is an embryonic stem cell (ESC) or an induced pluripotent stem cell (iPSC). In some embodiments, the method for achieving immune evasion includes genetically modifying a stem cell and differentiating the stem cell into the engineered cell. In some embodiments, differentiating the stem cell into the engineered cell includes contacting the stem cell with one or more differentiation factors. The specific combination of differentiation factors used will depend on the desired cell type(s). In some embodiments, differentiating the stem cell includes contacting the stem cell with one or more differentiation factors that drive the commitment and/or differentiation into a cardiac cell, a neural cell, a T cell, a retinal cell, an endocrine cell, an epithelial cell, a muscle cell, or a myeloid cell.

In some embodiments, the method for achieving immune evasion includes genetically modifying a cardiac cell, a neural cell, a T cell, a retinal cell, an endocrine cell, an epithelial cell, a muscle cell, or a myeloid cell. In some embodiments, the cardiac cell is a cell of the epicardium, the myocardium, or the endocardium of the heart. In some embodiments, the cardiac cell is a cardiomyocyte, a cardiac fibroblast, a cardiac smooth muscle cell, an epicardium cells, a cardiac endothelial cell, a Purkinje fiber, or a pacemaker cell. In some embodiments, the T cell is a helper T cell (CD4+ T cell), a cytotoxic T cell (CD8+ T cell), a memory T cell, a regulatory T cell (T reg cell), an innate-like T cell (e.g., a natural killer T cell), a mucosal-associated invariant T cell, or a gamma delta T cell, or a modified T cell (e.g., a CAR-T cell). In some embodiments, the retinal cell is a photoreceptor, a retinal horizontal cell, a retinal bipolar cell, a retinal amacrine cell, or a retinal ganglion cell. In some embodiments, the endocrine cell is a hypothalamus endocrine cell a pituitary gland endocrine cell, a pineal gland endocrine cell, a thyroid gland endocrine cell (e.g., a follicular cell), a parathyroid gland endocrine cell, a thymus gland endocrine cell, an adrenal gland endocrine cell, a pancreatic endocrine cell (e.g., an alpha cell, a beta cell, a delta cell or an F cell), an ovarian endocrine cell (e.g., a granulosa cell), or a testicular endocrine cell (e.g., a Leydig cell). In some embodiments, the epithelial cell is a squamous epithelial cell, a cuboidal epithelial cell, a columnar epithelial cell, a pseudostratified epithelia cell, or a stratified epithelial cell. In some embodiments, the muscle cell is a skeletal muscle cell, a cardiac muscle cell (e.g., a cardiomyocyte), or a smooth muscle cell. In some embodiments, the neural cell is a neuron or a glial cell. In some embodiments, the myeloid cell is a monocyte, a microglia, a macrophage, a dendritic cell, a basophil, an eosinophil, an erythrocyte, a mast cell, a neutrophil, a megakaryocyte, or a platelet, or any precursor progenitor cell thereof.

In some embodiments, the method for achieving immune evasion includes genetically modifying a human cell.

In some embodiments, the method for achieving immune evasion further includes measuring the immune evasion of the genetically modified cell. In some embodiments, the immune evasion includes reduced susceptibility to or inhibition of natural killer cell mediated cytotoxicity. In some embodiments, the immune evasion includes reduced susceptibility to or inhibition of T cell mediated cytotoxicity. Immune evasion can be measured by, for example, challenging the genetically modified cell with NK cells or T cells and measuring cell death, apoptosis or proliferation of the genetically modified cell in the presence of NK cells or T cells. Exemplary methods that can be used to measure the natural killer cell cytotoxicity include, but are not limited to a cell viability assay, an NK cell migration assay, a degranulation assay, a flow cytometry (FC)-based NK cytotoxicity assay, and any other suitable NK cell mediated cytotoxicity assay known in the art. Exemplary methods to measure T cell mediated cytotoxicity include, but are not limited to, a cell viability assay, a chromium (⁵¹Cr) release cytotoxicity assay, an IFNγ production assay, a annexin V binding cytotoxicity assay, and any other suitable T cell mediated cytotoxicity assay known in the art.

In some embodiments, the method for achieving immune evasion further includes expanding the genetically modified cell to produce a population of genetically modified cells.

### V. Kits and Dosage Forms

Certain aspects of the disclosure provide for kits for treating a disease or condition that includes a population of engineered cells, or a pharmaceutical composition that includes the population of engineered cells suitable for administration to a subject, including any of the populations of engineered cells and pharmaceutical compositions described herein.

In some embodiments, the kit includes instructional material for the use of said population of engineered cells or the pharmaceutical composition. In some embodiments, the instructional material includes instructions of preparing the pharmaceutical composition or the population of engineered cells for administration into a subject. Such instructions can include, but are not limited to, instructions for preparing or storing the population of engineered cells or the pharmaceutical composition, adding additives to the treatment, or combining the population of cells or the pharmaceutical composition with an additional therapeutic agent. In some embodiments, the instructional material includes instructions for administering the population of engineered cells or the pharmaceutical composition to a subject.

In some embodiments, the kit further includes an applicator for administering a population of engineered cells or a pharmaceutical composition that includes a population of engineered cells described herein. The applicator can be any device suitable for administration of the population of engineered cells or composition described herein to a subject, including, but not limited to, a hypodermic syringe, a needle, a balloon-dilating catheter, a pipette, and the like. The applicator can be a single-use or multiple-use administration device, and can be included in the kit as a pre-filled delivery system with, e.g., a pharmaceutical composition that includes the population of cells.

Certain aspects of the disclosure also provide for dosage forms of a pharmaceutical composition that includes a population of engineered cells suitable for administration to a subject. The dosage form can be in any form suitable for administration to a subject by any suitable route, including orally, pulmonarily, intranasally, parenterally (intravenously, intramuscularly, intraperitoneally, or subcutaneously), rectally, intralymphatically, intracranial, intracerebral or topically. In some embodiments, the dosage form is suitable for administration by injection into a subject.

### EXAMPLES

In order that this disclosure may be better understood, the following examples are set forth. These examples are for purposes of illustration only and are not to be construed as limiting the scope of the disclosure in any manner.

### Example 1: iPSC Screen

To identify innovative targets that could enable immune evasion from natural killer (NK) cells, an exhaustive literature search was conducted across diverse domains known for their relevance to immune evasion, including the tumor microenvironment, immune-privileged sites, stem cells, infections, the microbiome, peripheral tolerance, CRISPR screens, and transplantation sciences. From this thorough exploration, 78 potential gene targets were initially identified. Through rigorous triage based on the strength of supporting evidence, this list was distilled to a final selection of 12 gene targets, encompassing both cytoplasmic and membrane-bound genes. These gene targets were purposely chosen to encompass a broad spectrum of biological functions, encompassing the initiation of lymphocyte apoptosis, defense against lymphocyte-mediated apoptosis, and the initiation of inhibitory signaling pathways. This diverse array of selected gene targets significantly enhanced the potential for downstream multiplexing, i.e. engineering a cell with a combination of gene targets as described herein, of immune evasion strategies, offering benefits such as synergistic combinations and resistance prevention.

From these efforts, the specific gene targets listed in Tables 1-2 were selected for experimental investigation.

TABLE 1
Amino acid sequences of gene targets for achieving immune evasion.

| | | SEQ ID |
|---|---|---|
| Gene | Amino Acid Sequence | NO |
| VISTA | MGVPTALEAGSWRWGSLLFALFLAASLGPVAAFKVATPYS | 1 |
| | LYVCPEGQNVTLTCRLLGPVDKGHDVTFYKTWYRSSRGE | |
| | VQTCSERRPIRNLTFQDLHLHHGGHQAANTSHDLAQRHGL | |
| | ESASDHHGNFSITMRNLTLLDSGLYCCLVVEIRHHHSEHRV | |
| | HGAMELQVQTGKDAPSNCVVYPSSSQDSENITAAALATG | |
| | ACIVGILCLPLILLLVYKQRQAASNRRAQELVRMDSNIQGI | |
| | ENPGFEASPPAQGIPEAKVRHPLSYVAQRQPSESGRHLLSE | |
| | PSTPLSPPGPGDVFFPSLDPVPDSPNFEVI | |
| CLEC2D | MHDSNNVEKDITPSELPANPGCLHSKEHSIKATLIWRLFFLI | 2 |
| | MFLTIIVCGMVAALSAIRANCHQEPSVCLQAACPESWIGFQ | |
| | RKCFYFSDDTKNWTSSQRFCDSQDADLAQVESFQELNFLL | |
| | RYKGPSDHWIGLSREQGQPWKWINGTEWTRQFPILGAGE | |
| | CAYLNDKGASSARHYTERKWICSKSDIHV | |
| FASL | MQQPFNYPYPQIYWVDSSASSPWAPPGTVLPCPTSVPRRP | 3 |
| | GQRRPPPPPPPPPLPPPPPPPPLPPLPLPPLKKRGNHSTGLCL | |
| | LVMFFMVLVALVGLGLGMFQLFHLQKELAELRESTSQMH | |
| | TASSLEKQIGHPSPPPEKKELRKVAHLTGKSNSRSMPLEWE | |
| | DTYGIVLLSGVKYKKGGLVINETGLYFVYSKVYFRGQSCN | |
| | NLPLSHKVYMRNSKYPQDLVMMEGKMMSYCTTGQMWA | |
| | RSSYLGAVFNLTSADHLYVNVSELSLVNFEESQTFFGLYKL | |
| TRAIL | MAMMEVQGGPSLGQTCVLIVIFTVLLQSLCVAVTYVYFTN | 4 |
| | ELKQMQDKYSKSGIACFLKEDDSYWDPNDEESMNSPCWQ | |
| | VKWQLRQLVRKMILRTSEETISTVQEKQQNISPLVRERGPQ | |
| | RVAAHITGTRGRSNTLSSPNSKNEKALGRKINSWESSRSGH | |
| | SFLSNLHLRNGELVIHEKGFYYIYSQTYFRFQEEIKENTKN | |
| | DKQMVQYIYKYTSYPDPILLMKSARNSCWSKDAEYGLYSI | |
| | YQGGIFELKENDRIFVSVTNEHLIDMDHEASFFGAFLVG | |
| SERPINA3 | MERMLPLLALGLLAAGFCPAVLCHPNSPLDEENLTQENQD | 5 |
| | RGTHVDLGLASANVDFAFSLYKQLVLKAPDKNVIFSPLSIS | |
| | TALAFLSLGAHNTTLTEILKGLKFNLTETSEAEIHQSFQHLL | |
| | RTLNQSSDELQLSMGNAMFVKEQLSLLDRFTEDAKRLYGS | |
| | EAFATDFQDSAAAKKLINDYVKNGTRGKITDLIKDLDSQT | |
| | MMVLVNYIFFKAKWEMPFDPQDTHQSRFYLSKKKWVMV | |
| | PMMSLHHLTIPYFRDEELSCTVVELKYTGNASALFILPDQD | |
| | KMEEVEAMLLPETLKRWRDSLEFREIGELYLPKFSISRDYN | |
| | LNDILLQLGIEEAFTSKADLSGITGARNLAVSQVVHKAVLD | |
| | VFEEGTEASAATAVKITLLSALVETRTIVRFNRPFLMIIVPTD | |
| | TQNIFFMSKVTNPKQA | |
| SERPINB9 | METLSNASGTFAIRLLKILCQDNPSHNVFCSPVSISSALAM | 6 |
| | VLLGAKGNTATQMAQALSLNTEEDIHRAFQSLLTEVNKAG | |
| | TQYLLRTANRLFGEKTCQFLSTFKESCLQFYHAELKELSFI | |
| | RAAEESRKHINTWVSKKTEGKIEELLPGSSIDAETRLVLVN | |
| | AIYFKGKWNEPFDETYTREMPFKINQEEQRPVQMMYQEA | |
| | TFKLAHVGEVRAQLLELPYARKELSLLVLLPDDGVELSTV | |
| | EKSLTFEKLTAWTKPDCMKSTEVEVLLPKFKLQEDYDMES | |
| | VLRHLGIVDAFQQGKADLSAMSAERDLCLSKFVHKSFVE | |
| | VNEEGTEAAAASSCFVVAECCMESGPRFCADHPFLFFIRH | |
| | NRANSILFCGRFSSP | |
| CTLA4 | MACLGFQRHKAQLNLATRTWPCTLLFFLLFIPVFCKAMHV | 7 |
| | AQPAVVLASSRGIASFVCEYASPGKATEVRVTVLRQADSQ | |
| | VTEVCAATYMMGNELTFLDDSICTGTSSGNQVNLTIQGLR | |
| | AMDTGLYICKVELMYPPPYYLGIGNGTQIYVIDPEPCPDSD | |
| | FLLWILAAVSSGLFFYSFLLTAVSLSKMLKKRSPLTTGVYV | |
| | KMPPTEPECEKQFQPYFIPIN | |
| CD47 | MWPLVAALLLGSACCGSAQLLFNKTKSVEFTFCNDTVVIP | 8 |
| | CFVTNMEAQNTTEVYVKWKFKGRDIYTFDGALNKSTVPT | |
| | DFSSAKIEVSQLLKGDASLKMDKSDAVSHTGNYTCEVTEL | |
| | TREGETIIELKYRVVSWFSPNENILIVIFPIFAILLFWGQFGIK | |
| | TLKYRSGGMDEKTIALLVAGLVITVIVIVGAILFVPGEYSLK | |
| | NATGLGLIVTSTGILILLHYYVFSTAIGLTSFVIAILVIQVIAYI | |
| | LAVVGLSLCIAACIPMHGPLLISGLSILALAQLLGLVYMKF | |
| | VASNQKTIQPPRKAVEEPLNAFKESKGMMNDE | |
| HLA- | MSRSVALAVLALLSLSGLEAGAVDPLLALGGGGSGGGGSG | 9 |
| C*03: 04 | GGGSIQRTPKIQVYSRHPAENGKSNFLNCYVSGFHPSDIEV | |
| (GAVDPL | DLLKNGERIEKVEHSDLSFSKDWSFYLLYYTEFTPTEKDEY | |
| LAL (SEQ | ACRVNHVTLSQPKIVKWDRDMGGGGSGGGGSGGGGSGG | |
| ID NO: | GGSMGSHSMRYFYTAVSRPGRGEPHFIAVGYVDDTQFVRF | |
| 23)) | DSDAASPRGEPRAPWVEQEGPEYWDRETQKYKRQAQTD | |
| | RVSLRNLRGYYNQSEAGSHIIQRMYGCDVGPDGRLLRGY | |
| | DQYAYDGKDYIALNEDLRSWTAADTAAQITQRKWEAARE | |
| | AEQLRAYLEGLCVEWLRRYLKNGKETLQRAEHPKTHVTH | |
| | HPVSDHEATLRCWALGFYPAEITLTWQWDGEDQTQKAEL | |
| | VETRPAGDGTFQKWAAVVVPSGEEQRYTCHVQHEGLPEPL | |
| | TLRWEPSSQPTIPIVGIVAGLAVLAVLAVLGAVVAVVMCRR | |
| | KSSGGKGGSCSQAASSNSAQGSDESLIACKA | |
| HLA- | MSRSVALAVLALLSLSGLEAQYDDAVYKLGGGGSGGGGS | 10 |
| C*04: 01 | GGGGSIQRTPKIQVYSRHPAENGKSNFLNCYVSGFHPSDIE | |
| (QYDDAV | VDLLKNGERIEKVEHSDLSFSKDWSFYLLYYTEFTPTEKDE | |
| YKL (SEQ | YACRVNHVTLSQPKIVKWDRDMGGGGSGGGGSGGGGSG | |
| ID NO: | GGGSMGSHSMRYFSTSVSWPGRGEPRFIAVGYVDDTQFV | |
| 24)) | RFDSDAASPRGEPREPWVEQEGPEYWDRETQKYKRQAQA | |
| | DRVNLRKLRGYYNQSEDGSHTLQRMFGCDLGPDGRLLRG | |
| | YNQFAYDGKDYIALNEDLRSWTAADTAAQITQRKWEAAR | |
| | EAEQRRAYLEGTCVEWLRRYLENGKETLQRAEHPKTHVT | |
| | HHPVSDHEATLRCWALGFYPAEITLTWQWDGEDQTQKAE | |
| | LVETRPAGDGTFQKWAAVVVPSGEEQRYTCHVQHEGLPEP | |
| | LTLRWKPSSQPTIPIVGIVAGLAVLAVLAVLGAMVAVVMCR | |
| | RKSSGGKGGSCSQAASSNSAQGSDESLIACKA | |
| HLA- | MSRSVALAVLALLSLSGLEAIIDKSGIPVGGGGSGGGGSGG | 11 |
| C*05: 01 | GGSIQRTPKIQVYSRHPAENGKSNFLNCYVSGFHPSDIEVD | |
| (IIDKSGIP | LLKNGERIEKVEHSDLSFSKDWSFYLLYYTEFTPTEKDEYA | |
| V (SEQ ID | CRVNHVTLSQPKIVKWDRDMGGGGSGGGGSGGGGSGGG | |
| NO: 25)) | GSMCSHSMRYFYTAVSRPGRGEPRFIAVGYVDDTQFVQFD | |
| | SDAASPRGEPRAPWVEQEGPEYWDRETQKYKRQAQTDR | |
| | VNLRKLRGYYNQSEAGSHTLQRMYGCDLGPDGRLLRGY | |
| | NQFAYDGKDYIALNEDLRSWTAADKAAQITQRKWEAARE | |
| | AEQRRAYLEGTCVEWLRRYLENGKKTLQRAEHPKTHVTH | |
| | HPVSDHEATLRCWALGFYPAEITLTWQRDGEDQTQKAELV | |
| | ETRPAGDGTFQKWAAVVVPSGEEQRYTCHVQHEGLPEPLT | |
| | LRWGPSSQPTIPIVGIVAGLAVLAVLAVLGAVMAVVMCRRK | |
| | SSGGKGGSCSQAASSNSAQGSDESLIACKA | |

TABLE 2
Nucleic acid sequences of gene targets for achieving immune evasion.

| | | SEQ ID |
|---|---|---|
| Gene | Nucleic Acid Sequence | NO |
| VISTA | ATGGGGGTTCCCACTGCGCTCGAAGCGGGCTCCTGGAG | 12 |
| | ATGGGGTTCATTGCTTTTCGCTTTGTTTTTGGCGGCAAGC | |
| | CTCGGTCCAGTTGCGGCCTTCAAGGTTGCAACTCCATAC | |
| | TCCTTGTACGTCTGTCCCGAAGGTCAGAATGTCACCCTC | |
| | ACGTGTCGACTGCTGGGGCCTGTCGATAAAGGTCACGA | |
| | CGTGACGTTTTACAAGACCTGGTATCGATCATCAAGAGG | |
| | GGAAGTACAGACCTGTTCCGAGCGGCGGCCTATTAGGA | |
| | ACCTTACATTTCAAGACCTGCACTTGCACCACGGCGGAC | |
| | ATCAGGCCGCAAATACATCCCACGATCTGGCACAGCGGC | |
| | ATGGGCTTGAATCTGCTAGCGATCACCACGGGAATTTCT | |
| | CTATTACGATGCGGAATCTGACTCTCCTGGACTCAGGGC | |
| | TGTACTGCTGTTTGGTTGTAGAGATTCGCCACCATCATTC | |
| | AGAGCATAGGGTACACGGTGCGATGGAACTGCAAGTTC | |
| | AAACCGGTAAGGACGCTCCCAGCAACTGTGTCGTTTATC | |
| | CGAGCTCAAGCCAAGATAGCGAGAACATAACCGCCGCT | |
| | GCCCTTGCAACAGGGGCCTGTATCGTAGGGATTCTGTGC | |
| | CTGCCATTGATCCTCCTCCTTGTGTACAAACAGCGCCAG | |
| | GCAGCGTCTAATAGACGCGCCCAAGAGCTCGTCAGAAT | |
| | GGACAGTAATATCCAGGGCATAGAGAATCCAGGGTTTGA | |
| | AGCATCCCCTCCGGCGCAGGGGATTCCAGAAGCAAAAG | |
| | TTCGCCATCCTCTTAGCTACGTGGCCCAGCGACAACCCT | |
| | CTGAAAGTGGGAGACACCTGCTGTCTGAGCCCTCAACA | |
| | CCTCTGTCTCCGCCAGGTCCGGGAGATGTTTTTTTCCCAT | |
| | CCCTCGATCCTGTCCCTGATTCACCTAACTTTGAAGTTAT | |
| | A | |
| CLEC2D | ATGCACGATTCAAACAACGTGGAAAAAGACATTACCCC | 13 |
| | ATCCGAACTTCCAGCAAATCCTGGTTGCCTTCATAGCAA | |
| | GGAGCACTCTATCAAAGCAACACTGATTTGGCGCTTGTT | |
| | CTTCCTTATAATGTTTCTCACAATTATCGTTTGCGGTATGG | |
| | TAGCCGCCTTGAGCGCCATTCGGGCCAATTGCCACCAGG | |
| | AACCATCCGTATGCCTGCAAGCTGCTTGTCCCGAGAGCT | |
| | GGATTGGGTTTCAACGGAAGTGCTTTTACTTCAGTGACG | |
| | ATACCAAAAACTGGACATCATCTCAACGCTTCTGCGATA | |
| | GTCAAGACGCTGATCTGGCTCAGGTAGAGAGTTTCCAG | |
| | GAACTGAACTTTCTCCTCAGATATAAGGGTCCATCTGATC | |
| | ATTGGATTGGACTTTCTCGGGAACAGGGGCAACCTTGGA | |
| | AATGGATCAATGGTACGGAATGGACACGCCAGTTTCCTA | |
| | TACTCGGTGCAGGTGAATGTGCGTATCTTAATGACAAGG | |
| | GAGCGTCTTCTGCAAGGCACTATACAGAACGGAAATGG | |
| | ATATGTAGCAAGAGTGACATACACGTA | |
| FASL | ATGCAACAACCCTTCAATTATCCGTACCCCCAGATTTACT | 14 |
| | GGGTTGATTCTTCCGCTAGCAGTCCATGGGCTCCGCCGG | |
| | GAACAGTTTTGCCGTGCCCGACTTCTGTACCCAGACGAC | |
| | CTGGACAGCGAAGACCACCCCCCCCGCCTCCACCACCT | |
| | CCACTGCCACCACCGCCTCCTCCTCCCCCTCTCCCACCC | |
| | CTTCCATTGCCCCCGTTGAAGAAGCGAGGTAATCATTCA | |
| | ACTGGGTTGTGCCTGCTGGTAATGTTTTTTATGGTTCTTG | |
| | TAGCACTCGTCGGCCTTGGCCTTGGGATGTTCCAATTGTT | |
| | TCACCTTCAAAAGGAACTTGCCGAACTTCGCGAATCCAC | |
| | AAGTCAAATGCACACTGCTAGCAGTTTGGAGAAACAAA | |
| | TTGGGCATCCGTCCCCTCCTCCTGAGAAAAAAGAACTCA | |
| | GAAAAGTTGCGCATTTGACTGGGAAATCCAACTCTAGGA | |
| | GTATGCCACTTGAATGGGAGGATACATACGGAATAGTTCT | |
| | CCTCAGTGGCGTGAAGTACAAAAAGGGGGGACTTGTAA | |
| | TTAACGAAACCGGATTGTATTTTGTCTATAGTAAGGTGTA | |
| | TTTTCGAGGTCAAAGCTGCAACAACCTGCCGCTCAGCC | |
| | ACAAGGTATATATGAGAAATAGTAAGTACCCACAGGATC | |
| | TTGTCATGATGGAGGGTAAAATGATGTCATATTGCACGAC | |
| | AGGTCAGATGTGGGCCCGCTCCTCTTACCTGGGTGCGGT | |
| | ATTTAACTTGACTTCTGCAGATCACCTTTATGTGAATGTA | |
| | TCAGAGCTTTCTCTGGTGAACTTTGAGGAGAGTCAGAC | |
| | ATTTTTCGGACTTTATAAGTTG | |
| TRAIL | ATGGCGATGATGGAAGTACAAGGAGGTCCTAGCCTGGG | 15 |
| | ACAGACGTGTGTCCTGATAGTTATTTTCACCGTTCTTCTC | |
| | CAATCACTGTGTGTAGCAGTGACGTATGTATATTTTACAA | |
| | ATGAACTTAAACAAATGCAGGATAAGTACTCCAAGTCAG | |
| | GAATCGCTTGCTTTCTCAAAGAAGACGACAGCTACTGG | |
| | GACCCGAACGACGAGGAATCAATGAATTCACCATGCTG | |
| | GCAAGTCAAATGGCAGCTTCGGCAGCTGGTGAGGAAAA | |
| | TGATTCTTCGAACTAGTGAAGAAACAATTTCTACCGTAC | |
| | AGGAAAAACAACAAAACATATCTCCACTTGTCCGCGAA | |
| | CGAGGACCTCAGAGGGTAGCCGCACATATCACGGGTAC | |
| | ACGCGGTCGCTCAAATACCTTGTCCAGTCCCAATTCCAA | |
| | AAATGAAAAGGCATTGGGAAGGAAAATCAACTCCTGGG | |
| | AGTCTAGCAGGTCTGGTCATAGCTTCTTGAGTAATCTTCA | |
| | TCTCCGAAATGGTGAGCTCGTGATTCACGAAAAGGGCTT | |
| | CTACTATATATATTCTCAGACTTATTTCCGCTTTCAGGAGG | |
| | AGATCAAAGAAAATACAAAAAATGATAAGCAGATGGTG | |
| | CAATATATTTACAAATACACATCTTACCCAGACCCCATTTT | |
| | GCTGATGAAATCCGCACGCAACTCCTGCTGGTCTAAGGA | |
| | TGCTGAGTACGGATTGTATTCCATTTATCAGGGAGGCATA | |
| | TTTGAATTGAAAGAGAATGATCGAATATTTGTAAGCGTC | |
| | ACAAACGAGCATTTGATAGATATGGACCATGAAGCCTCA | |
| | TTTTTCGGTGCTTTCTTGGTGGGG | |
| SERPINA3 | ATGGAACGAATGCTTCCCCTGCTTGCTCTGGGGTTGCTG | 16 |
| | GCCGCTGGGTTTTGCCCGGCGGTATTGTGCCATCCAAAT | |
| | AGCCCATTGGACGAAGAAAATTTGACGCAAGAGAATCA | |
| | GGATAGAGGCACTCATGTGGACCTTGGCTTGGCCTCCGC | |
| | TAACGTCGATTTCGCATTTTCTCTCTATAAACAGCTGGTC | |
| | CTTAAAGCTCCCGATAAAAACGTAATATTTAGTCCGCTCA | |
| | GTATTTCAACGGCGCTCGCCTTCTTGTCTCTTGGGGCGC | |
| | ACAACACCACGTTGACGGAAATTCTGAAAGGACTTAAA | |
| | TTTAACCTCACCGAAACGTCTGAGGCCGAGATTCACCAA | |
| | TCATTCCAACACCTGCTCCGCACGTTGAATCAAAGCTCC | |
| | GATGAGCTTCAACTTTCTATGGGTAATGCGATGTTCGTGA | |
| | AGGAGCAACTTAGCTTGCTTGATAGGTTTACAGAAGATG | |
| | CGAAGAGGCTTTACGGAAGTGAGGCGTTCGCTACAGAC | |
| | TTCCAAGATTCAGCCGCGGCGAAGAAGCTCATCAATGAT | |
| | TACGTCAAGAATGGGACTAGAGGAAAAATAACTGACTT | |
| | GATTAAAGATCTGGATTCCCAAACGATGATGGTATTGGTC | |
| | AATTATATTTTCTTTAAGGCTAAGTGGGAAATGCCTTTTG | |
| | ATCCTCAAGATACCCACCAATCTAGATTCTACCTCTCTAA | |
| | AAAGAAATGGGTCATGGTACCTATGATGAGTCTGCACCA | |
| | TTTGACAATTCCGTATTTTCGGGACGAGGAGCTGTCATG | |
| | TACGGTAGTCGAGCTGAAATACACCGGCAATGCTTCCGC | |
| | CCTCTTCATACTTCCCGACCAGGATAAAATGGAAGAAGT | |
| | CGAAGCCATGCTTCTTCCGGAAACCCTTAAACGGTGGCG | |
| | AGACTCACTCGAATTTAGAGAAATAGGCGAGCTCTACCT | |
| | TCCAAAGTTTAGCATTTCACGAGATTACAACTTGAACGA | |
| | TATTCTCCTGCAATTGGGAATAGAGGAGGCTTTCACATCT | |
| | AAAGCGGATTTGAGTGGCATTACGGGCGCTAGAAATCTG | |
| | GCAGTGTCCCAGGTTGTTCACAAGGCAGTGTTGGATGTT | |
| | TTTGAGGAAGGAACCGAAGCAAGTGCCGCGACGGCAGT | |
| | CAAAATAACGCTCCTGAGCGCACTCGTTGAAACGAGAA | |
| | CGATAGTGCGGTTTAATAGACCGTTCCTGATGATCATAGT | |
| | CCCAACCGACACTCAGAACATTTTCTTTATGTCCAAGGT | |
| | GACTAACCCTAAGCAAGCG | |
| SERPINB9 | ATGGAAACACTGTCCAATGCGTCCGGTACCTTTGCTATC | 17 |
| | AGGCTCCTCAAGATTCTCTGTCAGGATAATCCTTCCCAC | |
| | AACGTTTTCTGCAGCCCTGTGAGTATAAGCAGTGCGCTT | |
| | GCAATGGTTCTGCTGGGCGCTAAAGGCAATACCGCAACC | |
| | CAAATGGCACAAGCCCTTAGTCTTAATACCGAGGAGGAC | |
| | ATCCACAGGGCATTCCAATCCTTGTTGACCGAAGTCAAT | |
| | AAGGCGGGGACCCAGTACTTGCTGCGCACGGCCAATAG | |
| | ACTGTTTGGCGAGAAAACTTGTCAATTTCTGTCAACATT | |
| | CAAAGAGTCTTGTCTGCAGTTTTACCACGCCGAGTTGAA | |
| | GGAACTTTCCTTTATCAGGGCGGCCGAGGAGTCTCGCAA | |
| | ACACATTAATACTTGGGTATCAAAAAAGACCGAAGGTAA | |
| | AATCGAGGAACTTCTTCCAGGATCCTCTATAGATGCCGA | |
| | AACACGGCTGGTGCTTGTGAACGCTATATACTTCAAAGG | |
| | AAAGTGGAACGAGCCATTTGATGAGACCTATACCCGCGA | |
| | AATGCCCTTCAAAATAAACCAGGAAGAGCAGCGGCCTG | |
| | TCCAGATGATGTACCAAGAGGCCACATTCAAGCTTGCCC | |
| | ACGTAGGAGAAGTGCGGGCGCAACTGCTGGAGCTGCCT | |
| | TACGCCAGGAAAGAGCTTAGTCTGCTCGTGCTGCTTCCC | |
| | GATGATGGGGTCGAACTTTCCACTGTTGAGAAATCCCTC | |
| | ACTTTTGAAAAGCTCACTGCCTGGACTAAGCCGGATTGC | |
| | ATGAAGAGTACGGAAGTCGAAGTGTTGCTGCCCAAATTT | |
| | AAGCTTCAAGAAGACTACGATATGGAGTCCGTTCTCCGA | |
| | CATTTGGGAATCGTCGATGCGTTCCAGCAGGGGAAAGCT | |
| | GATTTGAGCGCCATGTCTGCCGAAAGGGACCTGTGCCTC | |
| | TCTAAGTTCGTCCATAAGTCTTTTGTTGAAGTAAACGAA | |
| | GAGGGCACCGAAGCGGCCGCGGCATCAAGTTGTTTCGT | |
| | AGTAGCCGAGTGCTGCATGGAGAGCGGACCGAGATTCT | |
| | GCGCAGACCATCCGTTTCTCTTTTTTATCAGACACAACC | |
| | GGGCGAACTCAATTCTGTTTTGCGGTAGGTTCTCTTCCC | |
| | CA | |
| CTLA4 | ATGGCTTGTCTGGGTTTCCAGAGACATAAGGCCCAACTG | 18 |
| | AATTTGGCCACGCGAACGTGGCCGTGCACGCTTTTGTTT | |
| | TTCTTGCTTTTTATCCCGGTCTTTTGCAAAGCAATGCACG | |
| | TGGCACAACCGGCTGTCGTACTGGCCTCAAGCAGAGGA | |
| | ATTGCAAGTTTTGTTTGTGAGTACGCCTCCCCCGGCAAG | |
| | GCTACTGAAGTCAGAGTTACTGTACTTCGCCAGGCTGAT | |
| | TCTCAAGTCACCGAGGTATGCGCCGCCACTTATATGATG | |
| | GGGAATGAACTGACATTTCTTGACGATTCCATTTGTACTG | |
| | GGACTTCCTCAGGCAATCAGGTAAACTTGACTATCCAGG | |
| | GCTTGAGAGCCATGGATACTGGTCTTTATATCTGTAAGGT | |
| | GGAACTCATGTACCCGCCCCCTTACTATTTGGGAATTGGT | |
| | AACGGAACCCAGATCTATGTGATAGACCCTGAACCTTGT | |
| | CCCGATTCAGATTTCCTGCTTTGGATTTTGGCGGCTGTAA | |
| | GTAGTGGCTTGTTCTTTTATAGTTTCTTGCTTACTGCGGT | |
| | ATCCCTCTCAAAGATGCTGAAGAAACGCTCTCCCTTGAC | |
| | AACTGGCGTATATGTGAAAATGCCCCCCACTGAGCCGGA | |
| | GTGTGAAAAGCAGTTCCAGCCTTACTTTATCCCGATTAA | |
| | C | |
| CD47 | ATGTGGCCTCTCGTGGCTGCGCTTCTGCTCGGTAGTGCT | 19 |
| | TGTTGCGGTTCCGCGCAGCTCCTTTTTAATAAGACCAAA | |
| | AGCGTAGAGTTCACCTTTTGTAATGATACTGTCGTTATTC | |
| | CCTGCTTCGTAACGAACATGGAAGCCCAAAATACAACTG | |
| | AGGTATATGTTAAATGGAAGTTCAAGGGCCGCGATATCTA | |
| | CACCTTCGATGGTGCTTTGAACAAGAGCACTGTACCAAC | |
| | CGATTTCAGCAGTGCTAAGATTGAGGTTAGCCAGCTTTT | |
| | GAAGGGAGACGCCTCACTGAAAATGGACAAGTCCGACG | |
| | CAGTTTCCCACACGGGCAATTATACTTGTGAAGTAACCG | |
| | AGCTCACTAGAGAAGGAGAAACGATAATCGAACTGAAA | |
| | TATCGCGTGGTGAGCTGGTTTAGTCCAAATGAGAATATAC | |
| | TGATCGTAATCTTTCCAATCTTCGCTATACTGCTTTTTTGG | |
| | GGTCAGTTTGGAATTAAGACGCTCAAGTATCGGTCAGGG | |
| | GGCATGGATGAAAAGACCATTGCCCTGCTGGTTGCGGG | |
| | ACTGGTTATTACGGTAATCGTCATTGTGGGTGCGATCCTT | |
| | TTTGTGCCGGGCGAATATAGTCTTAAAAACGCGACGGGT | |
| | TTGGGCTTGATTGTAACATCTACCGGCATCCTTATCCTGC | |
| | TGCACTACTACGTCTTTTCTACAGCTATTGGACTGACCAG | |
| | CTTTGTCATCGCAATTCTGGTGATTCAGGTAATTGCGTAT | |
| | ATACTTGCAGTAGTAGGTCTTTCTTTGTGTATAGCGGCCT | |
| | GCATTCCTATGCACGGACCGCTCTTGATAAGCGGACTGT | |
| | CCATACTCGCTCTCGCACAGCTGCTTGGACTTGTTTATAT | |
| | GAAGTTTGTCGCTTCCAATCAGAAAACGATTCAGCCACC | |
| | CAGAAAGGCCGTGGAAGAACCATTGAACGCCTTCAAGG | |
| | AGTCTAAAGGAATGATGAATGATGAG | |
| HLA- | ATGTCTCGCTCCGTGGCCTTAGCTGTGCTCGCGCTACTCT | 20 |
| C*03: 04 | CTCTTTCTGGCCTCGAGGCTGGGGCGGTAGACCCGCTGT | |
| (GAVDPL | TGGCGCTTGGTGGTGGCGGATCCGGTGGTGGCGGTTCTG | |
| LAL (SEQ | GTGGTGGCGGCTCCATCCAGCGTACGCCAAAGATTCAG | |
| ID NO: | GTTTACTCACGTCATCCAGCAGAGAATGGAAAGTCAAAT | |
| 23)) | TTCCTGAATTGCTATGTGTCTGGGTTTCATCCATCCGACA | |
| | TTGAAGTTGACTTACTGAAGAATGGAGAGAGAATTGAA | |
| | AAAGTGGAGCATTCAGACTTGTCTTTCAGCAAGGACTG | |
| | GTCTTTCTATCTCTTGTACTACACTGAATTCACCCCCACT | |
| | GAAAAAGATGAGTATGCCTGCCGTGTGAACCATGTGACT | |
| | TTGTCACAGCCCAAGATAGTTAAGTGGGATCGCGACATG | |
| | GGTGGTGGCGGTTCTGGTGGTGGCGGTAGTGGCGGCGG | |
| | AGGAAGCGGTGGTGGCGGTTCCATGGGCTCCCACTCCAT | |
| | GAGGTATTTCTACACCGCTGTGTCCCGGCCCGGCCGCGG | |
| | GGAGCCCCACTTCATCGCAGTGGGCTACGTGGACGACA | |
| | CGCAGTTCGTGCGGTTCGACAGCGACGCCGCGAGTCCG | |
| | AGAGGGGAGCCGCGGGCGCCGTGGGTGGAGCAGGAGG | |
| | GGCCGGAGTATTGGGACCGGGAGACACAGAAGTACAAG | |
| | CGCCAGGCACAGACTGACCGAGTGAGCCTGCGGAACCT | |
| | GCGCGGCTACTACAACCAGAGCGAGGCCGGGTCTCACA | |
| | TCATCCAGAGGATGTATGGCTGCGACGTGGGGCCCGACG | |
| | GGCGCCTCCTCCGCGGGTATGACCAGTACGCCTACGACG | |
| | GCAAGGATTACATCGCCCTGAACGAGGATCTGCGCTCCT | |
| | GGACCGCCGCGGACACGGCGGCTCAGATCACCCAGCGC | |
| | AAGTGGGAGGCGGCCCGTGAGGCGGAGCAGCTGAGAG | |
| | CCTACCTGGAGGGCCTGTGCGTGGAGTGGCTCCGCAGA | |
| | TACCTGAAGAATGGGAAGGAGACGCTGCAGCGCGCGGA | |
| | ACACCCAAAGACACACGTGACCCACCATCCCGTCTCTG | |
| | ACCATGAGGCCACCCTGAGGTGCTGGGCCCTGGGCTTCT | |
| | ACCCTGCGGAGATCACACTGACCTGGCAGTGGGATGGG | |
| | GAGGACCAAACTCAGAAAGCGGAGCTTGTGGAGACCA | |
| | GGCCAGCAGGAGATGGAACCTTCCAGAAGTGGGCAGCT | |
| | GTGGTGGTGCCTTCTGGAGAAGAGCAGAGATACACGTG | |
| | CCATGTGCAGCACGAGGGGCTGCCGGAGCCCCTCACCC | |
| | TGAGATGGGAGCCGTCTTCCCAGCCCACCATCCCCATCG | |
| | TGGGCATCGTTGCTGGCCTGGCTGTCCTGGCTGTCCTAG | |
| | CTGTCCTAGGAGCTGTGGTGGCTGTTGTGATGTGTAGGA | |
| | GGAAGAGCTCAGGTGGAAAAGGAGGGAGCTGCTCTCA | |
| | GGCTGCGTCCAGCAACAGTGCCCAGGGCTCTGATGAGT | |
| | CTCTCATCGCTTGTAAAGCC | |
| HLA- | ATGTCTCGCTCCGTGGCCTTAGCTGTGCTCGCGCTACTCT | 21 |
| C*04: 01 | CTCTTTCTGGCCTCGAGGCTCAATACGATGATGCGGTGT | |
| (QYDDAV | ACAAGTTGGGTGGTGGCGGATCCGGTGGTGGCGGTTCT | |
| YKL (SEQ | GGTGGTGGCGGCTCCATCCAGCGTACGCCAAAGATTCA | |
| ID NO: | GGTTTACTCACGTCATCCAGCAGAGAATGGAAAGTCAA | |
| 24)) | ATTTCCTGAATTGCTATGTGTCTGGGTTTCATCCATCCGA | |
| | CATTGAAGTTGACTTACTGAAGAATGGAGAGAGAATTGA | |
| | AAAAGTGGAGCATTCAGACTTGTCTTTCAGCAAGGACT | |
| | GGTCTTTCTATCTCTTGTACTACACTGAATTCACCCCCAC | |
| | TGAAAAAGATGAGTATGCCTGCCGTGTGAACCATGTGAC | |
| | TTTGTCACAGCCCAAGATAGTTAAGTGGGATCGCGACAT | |
| | GGGTGGTGGCGGTTCTGGTGGTGGCGGTAGTGGCGGCG | |
| | GAGGAAGCGGTGGTGGCGGTTCCATGGGCTCCCACTCC | |
| | ATGAGGTATTTCTCCACATCCGTGTCCTGGCCCGGCCGC | |
| | GGGGAGCCCCGCTTCATCGCAGTGGGCTACGTGGACGA | |
| | CACGCAGTTCGTGCGGTTCGACAGCGACGCCGCGAGTC | |
| | CAAGAGGGGAGCCGCGGGAGCCGTGGGTGGAGCAGGA | |
| | GGGGCCGGAGTATTGGGACCGGGAGACACAGAAGTACA | |
| | AGCGCCAGGCACAGGCTGACCGAGTGAACCTGCGGAA | |
| | ACTGCGCGGCTACTACAACCAGAGCGAGGACGGGTCTC | |
| | ACACCCTCCAGAGGATGTTTGGCTGCGACCTGGGGCCG | |
| | GACGGGCGCCTCCTCCGCGGGTATAACCAGTTCGCCTAC | |
| | GACGGCAAGGATTACATCGCCCTGAACGAGGATCTGCG | |
| | CTCCTGGACCGCCGCGGACACGGCGGCTCAGATCACCC | |
| | AGCGCAAGTGGGAGGCGGCCCGTGAGGCGGAGCAGCG | |
| | GAGAGCCTACCTGGAGGGCACGTGCGTGGAGTGGCTCC | |
| | GCAGATACCTGGAGAACGGGAAGGAGACGCTGCAGCGC | |
| | GCGGAACACCCAAAGACACACGTGACCCACCATCCCGT | |
| | CTCTGACCATGAGGCCACCCTGAGGTGCTGGGCCCTGG | |
| | GCTTCTACCCTGCGGAGATCACACTGACCTGGCAGTGGG | |
| | ATGGGGAGGACCAAACTCAGAAAGCGGAGCTTGTGGAG | |
| | ACCAGGCCAGCAGGAGATGGAACCTTCCAGAAGTGGGC | |
| | AGCTGTGGTGGTGCCTTCTGGAGAAGAGCAGAGATACA | |
| | CGTGCCATGTTCAGCACGAGGGGCTGCCGGAGCCCCTC | |
| | ACCCTGAGATGGAAGCCGTCTTCCCAGCCCACCATCCCC | |
| | ATCGTGGGCATCGTTGCTGGCCTGGCTGTCCTGGCTGTC | |
| | CTAGCTGTCCTAGGAGCTATGGTGGCTGTTGTGATGTGTA | |
| | GGAGGAAGAGCTCAGGTGGAAAAGGAGGGAGCTGCTC | |
| | TCAGGCTGCGTCCAGCAACAGTGCCCAGGGCTCTGATG | |
| | AGTCTCTCATCGCTTGTAAAGCC | |
| HLA- | ATGTCTCGCTCCGTGGCCTTAGCTGTGCTCGCGCTACTCT | 22 |
| C*05: 01 | CTCTTTCTGGCCTCGAGGCTATTATAGACAAGAGCGGCA | |
| (IIDKSGIP | TTCCGGTGGGTGGTGGCGGATCCGGTGGTGGCGGTTCTG | |
| V (SEQ ID | GTGGTGGCGGCTCCATCCAGCGTACGCCAAAGATTCAG | |
| NO: 25)) | GTTTACTCACGTCATCCAGCAGAGAATGGAAAGTCAAAT | |
| | TTCCTGAATTGCTATGTGTCTGGGTTTCATCCATCCGACA | |
| | TTGAAGTTGACTTACTGAAGAATGGAGAGAGAATTGAA | |
| | AAAGTGGAGCATTCAGACTTGTCTTTCAGCAAGGACTG | |
| | GTCTTTCTATCTCTTGTACTACACTGAATTCACCCCCACT | |
| | GAAAAAGATGAGTATGCCTGCCGTGTGAACCATGTGACT | |
| | TTGTCACAGCCCAAGATAGTTAAGTGGGATCGCGACATG | |
| | GGTGGTGGCGGTTCTGGTGGTGGCGGTAGTGGCGGCGG | |
| | AGGAAGCGGTGGTGGCGGTTCCATGTGCTCCCACTCCAT | |
| | GAGGTATTTCTACACCGCCGTGTCCCGGCCCGGCCGCGG | |
| | AGAGCCCCGCTTCATCGCAGTGGGCTACGTGGACGACA | |
| | CGCAGTTCGTGCAGTTCGACAGCGACGCCGCGAGTCCA | |
| | AGAGGGGAGCCGCGGGCGCCGTGGGTGGAGCAGGAGG | |
| | GGCCGGAGTATTGGGACCGGGAGACACAGAAGTACAAG | |
| | CGCCAGGCACAGACTGACCGAGTGAACCTGCGGAAACT | |
| | GCGCGGCTACTACAACCAGAGCGAGGCCGGGTCTCACA | |
| | CCCTCCAGAGGATGTATGGCTGCGACCTGGGGCCCGAC | |
| | GGGCGCCTCCTCCGCGGGTATAACCAGTTCGCCTACGAC | |
| | GGCAAGGATTACATCGCCCTGAATGAGGACCTGCGCTCC | |
| | TGGACCGCCGCGGACAAGGCGGCTCAGATCACCCAGCG | |
| | CAAGTGGGAGGCGGCCCGTGAGGCGGAGCAGCGGAGA | |
| | GCCTACCTGGAGGGCACGTGCGTGGAGTGGCTCCGCAG | |
| | ATACCTGGAGAACGGGAAGAAGACGCTGCAGCGCGCGG | |
| | AACACCCAAAGACACACGTGACCCACCATCCCGTCTCT | |
| | GACCATGAGGCCACCCTGAGGTGCTGGGCCCTGGGCTT | |
| | CTACCCTGCGGAGATCACACTGACCTGGCAGCGGGATG | |
| | GCGAGGACCAAACTCAGAAAGCGGAGCTTGTGGAGAC | |
| | CAGGCCAGCAGGAGATGGAACCTTCCAGAAGTGGGCAG | |
| | CTGTGGTGGTGCCTTCTGGAGAAGAGCAGAGATACACG | |
| | TGCCATGTGCAGCACGAGGGGCTGCCAGAGCCCCTCAC | |
| | CCTGAGATGGGGGCCATCTTCCCAGCCCACCATCCCCAT | |
| | CGTGGGCATCGTTGCTGGCCTGGCTGTCCTGGCTGTCCT | |
| | AGCTGTCCTAGGAGCTGTGATGGCTGTTGTGATGTGTAG | |
| | GAGGAAGAGCTCAGGTGGAAAAGGAGGGAGCTGCTCT | |
| | CAGGCTGCGTCCAGCAACAGTGCCCAGGGCTCTGATGA | |
| | GTCTCTCATCGCTTGTAAAGCC | |

Building upon the selection process, and without being bound by theory, it was contemplated that if one or more of the gene targets were upregulated in induced pluripotent stem cells (iPSCs), they might hinder or inhibit the cytotoxicity mediated by NK cells. To test this, iPSCs were genetically engineered to express each gene target by introducing nucleic acids encoding the gene target and subjecting the engineered iPSCs to NIK cell toxicity assays.

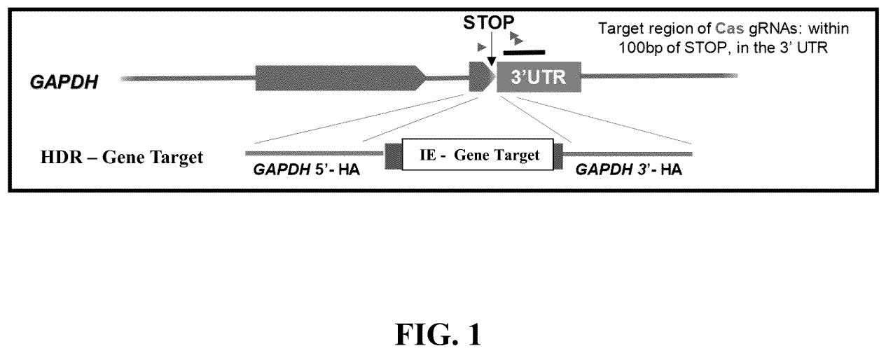

FIG. 1 illustrates the strategy employed to genetically engineer iPSCs with potential gene targets for NK cell evasion. Briefly, heterologous nucleic acids encoding the gene targets listed Table 1 and Table 2 were introduced into iPSCs using a CRISPR gene editing system. Specifically, the system included Cas proteins that were pre-complexed with guide RNAs specific to the GAPDH locus to form ribonucleoparticles (RNPs), which were then electroporated alongside a homology-directed DNA repair (HDR) template into the iPSCs. This strategy resulted in the integration of the heterologous nucleic acids encoding each gene targets downstream of an endogenous STEL promoter.

Before integrating the heterologous nucleic acids encoding gene targets into the genome of the iPSCs, an analysis to identify an optimal integration site was conducted. One factor that was considered was the ability of transgenes to exhibit stable expression throughout extended cell culture (e.g., >5 cell divisions) and differentiation into downstream cell types, such as cardiomyocytes. As a result, the GAPDH STEL site was selected, building upon previously described work (WO2021072329A1, which is incorporated by reference in its entirety).

After integration, the expression of each immune evasion gene target was regulated by an endogenous gene promoter, specifically the GAPDH promoter. Consequently, the expression of the gene target was linked to the expression of the endogenous GAPDH gene. This design ensured that the expression of the immune evasion gene targets remain sustained and constitutive, as the GAPDH gene is essential in cellular metabolism and consistently expressed. Surface expression of each immune evasion gene target was confirmed by flow cytometry analysis.

Nucleic Acid sequences encoding each of the gene targets identified in Tables 1-2 were individually integrated into separate iPSC lines to prevent cross-interference and enable isolated assessment of each gene's role in immune evasion. The engineered cells were subsequently expanded and cryopreserved for further study.

### Example 2: Immunoassay of Engineered iPSCs with Natural Killer Cells

To evaluate the impact of the gene targets identified in Tables 1-2 in achieving evasion from NK cells, a series of immunoassays were conducted, in vitro, in which the engineered iPSCs of Example 1 were directly exposed to NK cells from different healthy donors. The percentage of the engineered iPSCs that remained viable after encountering NK cells was measured and used as a metric for inhibition of NK cell mediated cytotoxicity. A higher survival rate would be indicative of a more effective immune evasion, and would show the utility of the introduced gene target for immune evasion of therapeutic cells.

In these NK cell cytotoxicity assays, engineered iPSCs were co-cultured with NK cells from three different healthy donors (i.e., pNK1, pNK2, and pNK3). Each NK cell batch used originated from a different donor to account for the potential for inter-individual variability in NK cell behavior and responsiveness. The diversity among donors also allowed an assessment of the robustness and consistency of the immune evasion strategy across a range of genetic backgrounds, helping to ensure its broader applicability and effectiveness.

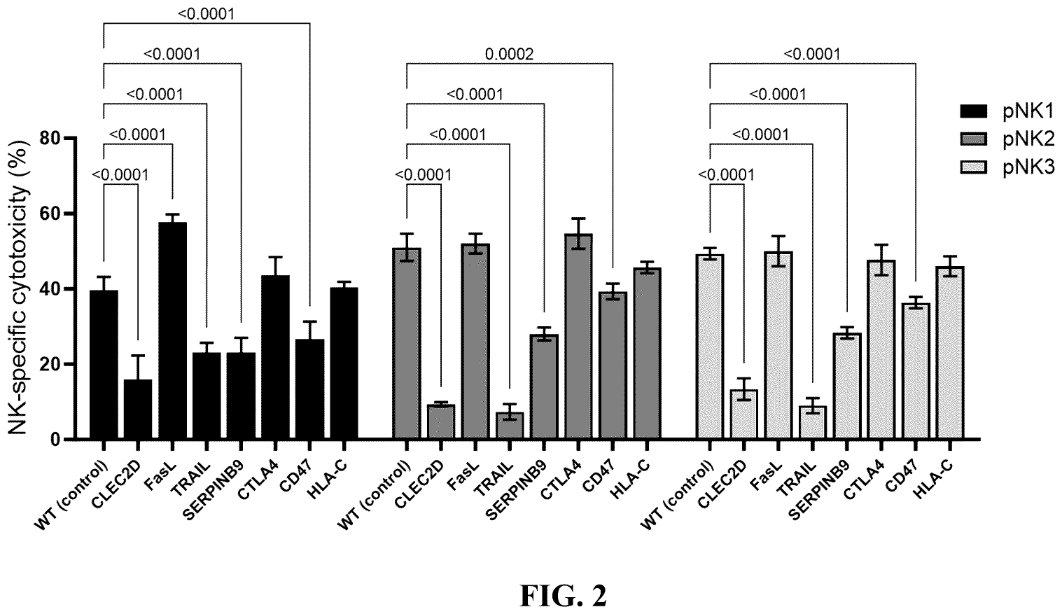

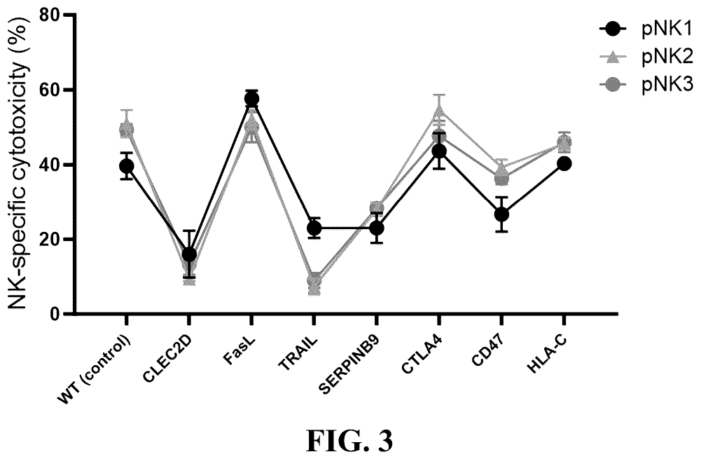

FIGS. 2 and 3 show exemplary experimental results from the NK cell cytotoxicity assays. In FIG. 2, the data is presented as a bar graph showing that iPSCs expressing CLEC2D, TRAIL, SERPINB9 or CD47 demonstrated the ability to inhibit NK cell-mediated cytotoxicity, as evidenced by reduced NK cell-mediated toxicity (indicated along y-axis) compared with control cells, i.e., iPSCs which have not been engineered with the recited gene targets. FIG. 3 presents this data as a line graph to visualize the consistency of responses across different NK cell populations. The line graph format emphasizes the robust nature of certain gene targets, in particular CLEC2D, TRAIL, and SERPINB9, in achieving evasion from natural killer cells across various NK cell populations.

The results of the NK cell cytotoxicity assays demonstrated that upregulation of the gene targets listed in Tables 1-2 is a viable and novel strategy for immune evasion in therapeutic cells.

### Example 3: Differentiation of Engineered iPSCs into Cardiomyocytes and NK Cell Challenge

To assess the capability of engineered iPSCs to differentiate into cardiomyocytes after genetic manipulation, including upregulation of CLEC2D, TRAIL, SERPINB9, and two HLA-C variants (HLA-C*05:01 (IIDKSGIPV (SEQ ID NO: 25)), and HLA-C*04:01 (QYDDAVYKL (SEQ ID NO: 24)), engineered iPSCs were subjected to a cardiomyocyte differentiation protocol. Gene expression profiling of the resultant cardiomyocytes was conducted to confirm their successful differentiation, focusing on specific markers (e.g., cardiac troponin T (cTNT)) indicative of cardiomyocyte identity and functionality. For example, flow cytometry analysis confirmed that greater than 99% of the cardiomyocytes that were differentiated from engineered iPSCs edited for upregulation of CLEC2D were positive for cTNT expression.

Following successful differentiation, the engineered cardiomyocytes were also evaluated by flow cytometry analysis to confirm the engineered cells remained positive for the surface expression of gene targets (CLEC2D, TRAIL, SERPINB9, and two HLA-C variants (HLA-C*05:01 (IIDKSGIPV (SEQ ID NO: 25)), or HLA-C*04:01 (QYDDAVYKL (SEQ ID NO: 24))). Subsequently, the engineered cardiomyocytes were subjected to challenges by NK cells from multiple healthy donors, replicating the immunoassays detailed in Example 2. This testing was important to assess whether the observed immune evasion properties in the engineered iPSCs extended to the differentiated cardiomyocytes.

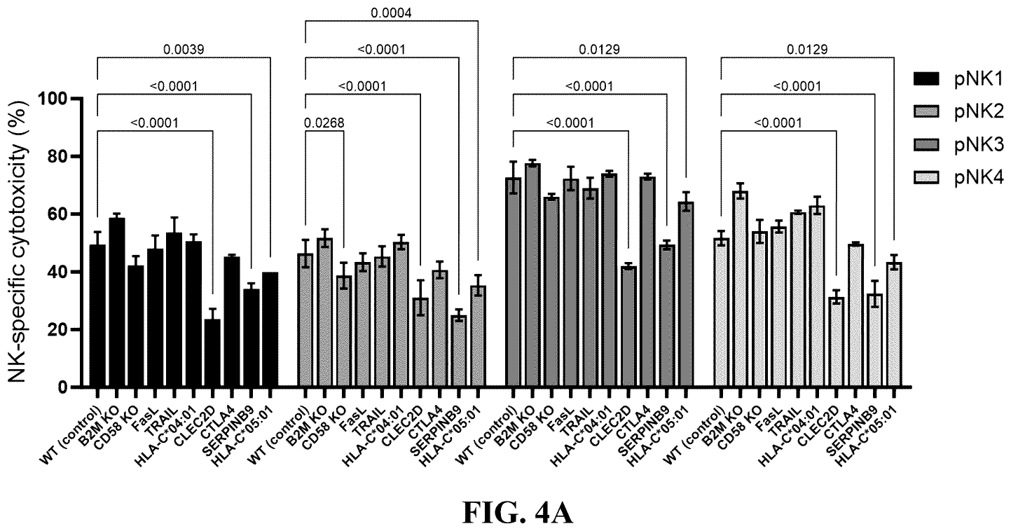

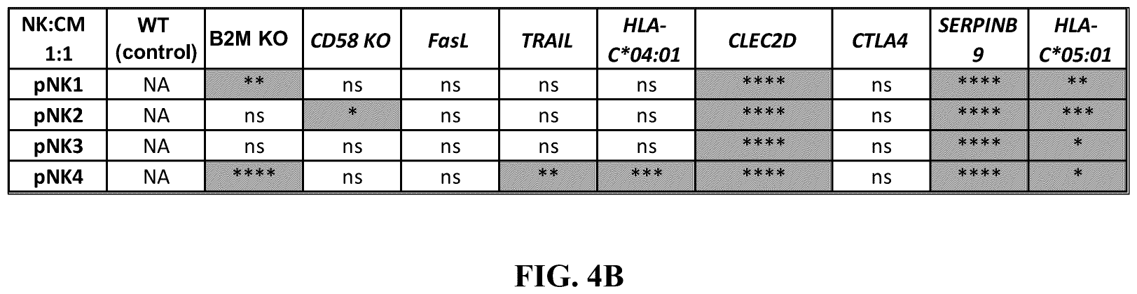

FIGS. 4A-4B shows exemplary experimental results from the NK cell cytotoxicity assays conducted with engineered cardiomyocytes. The data show engineered cardiomyocytes expressing CLEC2D, SERPINB9, and HLA-C*05:01 (IIDKSGIPV (SEQ ID NO: 25)) demonstrated the ability to inhibit NK cell-mediated cytotoxicity in all four pNK donors. This was evidenced by reduced NK cell-mediated toxicity (indicated along y-axis) in these cell populations as compared with control cells, i.e., cardiomyocytes which have not been engineered with the recited gene targets.

Notably, some genes effectively reduced cytotoxicity in engineered iPSCs did not exhibit the same level of efficacy in differentiated cardiomyocytes. This discrepancy underscores the importance of the described assays, highlighting that the success of a gene target in one cellular context does not guarantee similar results in another, reinforcing the unique and context-dependent nature of the insights described herein. Moreover, the level of expression of these gene targets may influence their ability to confer protection against NK cell-mediated cytotoxicity.

### Example 4: Cardiomyocytes Engineered with CLEC2D or SERPINB9 Successfully Evade NK Cell Mediated Rejection In Vivo

This Example describes experiments that were conducted to evaluate the ability of cardiomyocytes engineered to express novel gene targets (CLEC2D or SERPINB9) to evade NK cell mediated rejection in vivo. Heterologous nucleic acids encoding CLEC2D (SEQ ID NO: 2) or SERPINB9 (SEQ ID NO: 6) were incorporated into separate iPSC lines that had been previously engineered to express luciferase. The iPSCs were engineered to express either CLEC2D or SERPINB9 using a CRISPR gene editing system to integrate the corresponding heterologous nucleic acids into GAPDH STEL sites as described in Example 1. Luciferase expression by the engineered cells enabled the visualization, quantification, and tracking of CLEC2D and SERPINB9 engineered cardiomyocytes in vivo.

Following engineering, populations of engineered iPSCs expressing CLEC2D or SERPINB9, together with luciferase-positive control iPSCs, were differentiated into cardiomyocytes as described herein. Flow cytometry analysis confirmed successful differentiation of the iPSCs into cardiomyocyte (data not shown). These engineered cardiomyocytes were then transplanted into the hindlimbs of humanized mice that had been depleted of NK cells. Transplantation was performed either with the engineered cells alone or alongside NK cells sourced from two different donors (Donor 1 and Donor 2).

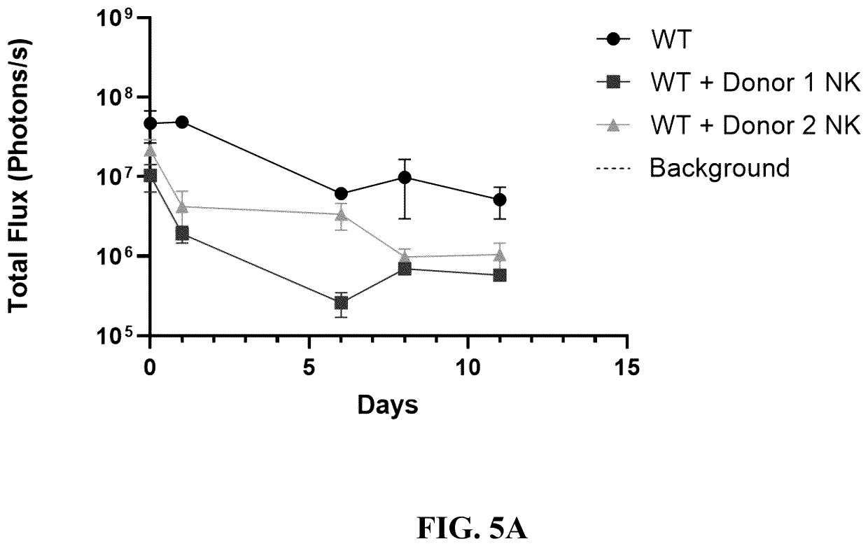

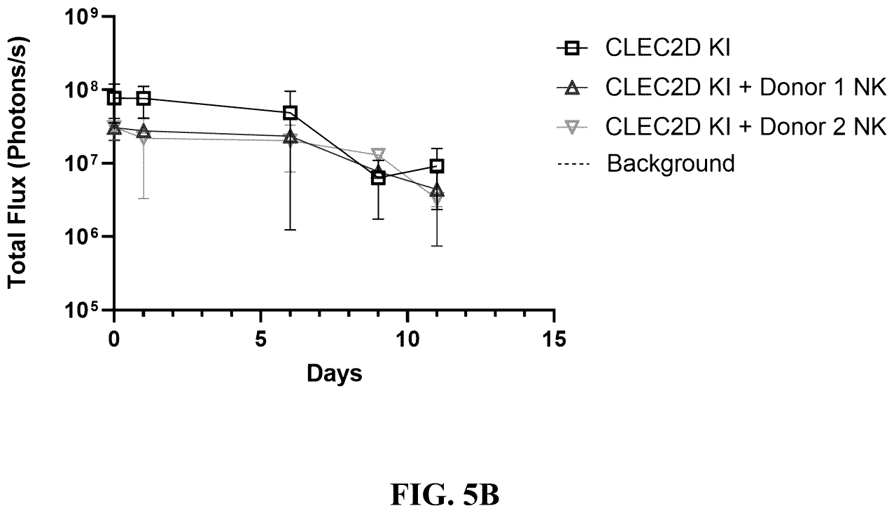

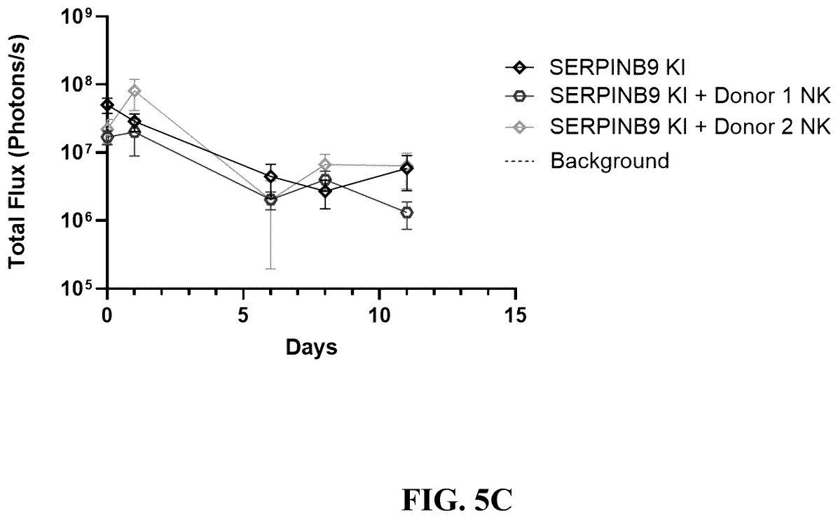

FIGS. 5A-5C are exemplary experimental results demonstrating the ability of engineered cardiomyocytes expressing CLEC2D or SERPINB9 to evade NK cell-mediated toxicity in vivo. Co-transplantation of control cardiomyocytes with Donor 1 NK cells or Donor 2 NK cells in vivo led to a significant reduction in the quantity of transplanted cardiomyocytes over time as indicated by a decrease in total photon flux from luciferase positive cells (FIG. 5A). Conversely, engineered cardiomyocytes expressing CLEC2D or SERPINB9, with and without co-transplantation of donor NK cells, exhibited a consistent photon flux (FIGS. 5B and 5C), demonstrating the ability of these gene targets to aid the cardiomyocytes in evading NK cell-mediated toxicity from two different donors in vivo.

Taken together, these data show cardiomyocytes engineered to have increased expression of CLEC2D or SERPINB9, as compared with control cells, demonstrated the ability to evade NK mediated killing by multiple donors in vivo.

### Example 5: Evaluation of NK Cell Cytotoxicity Against iPSCs Engineered with Multiple Immune Evasion Genes

This example describes experiments that were conducted to evaluate the ability of iPSCs engineered to express multiple immune evasion genes to evade NK cell-mediated cytotoxicity in vitro.

iPSCs were engineered to express CLEC2D, SERPINB9, or TRAIL individually, as well as in combinations (CLEC2D+SERPINB9 and CLEC2D+TRAIL), using the CRISPR gene editing system to integrate the corresponding heterologous nucleic acids into GAPDH STEL sites as described in Example 1. Control iPSCs without any genetic modifications and iPSCs with a B2M knockout were also included.

The engineered iPSCs were subjected to in vitro NK cell cytotoxicity assays as described in Example 2. These cells were co-cultured with NK cells from multiple healthy donors, and the percentage of NK-specific cytotoxicity was measured.

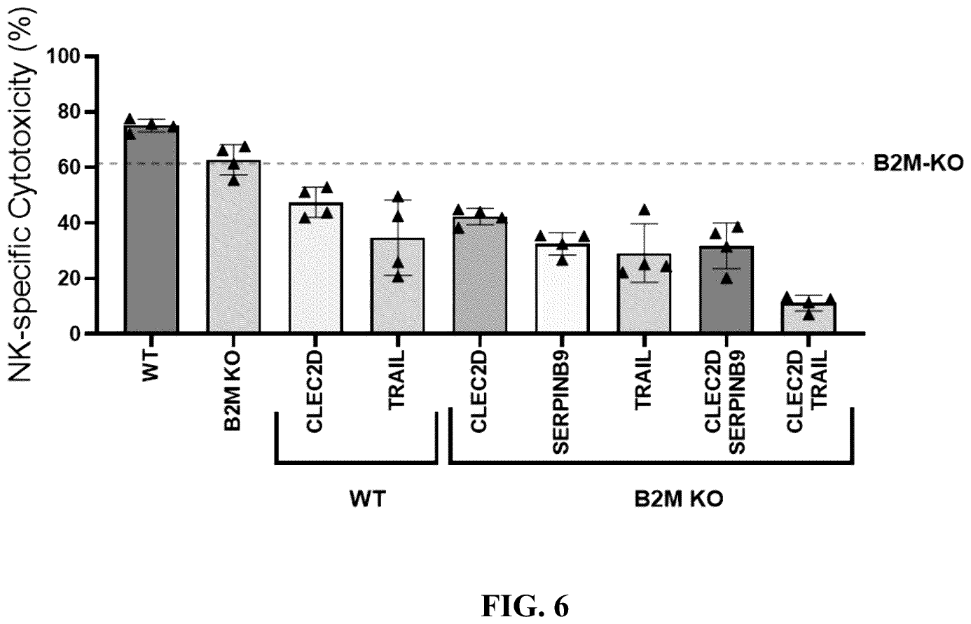

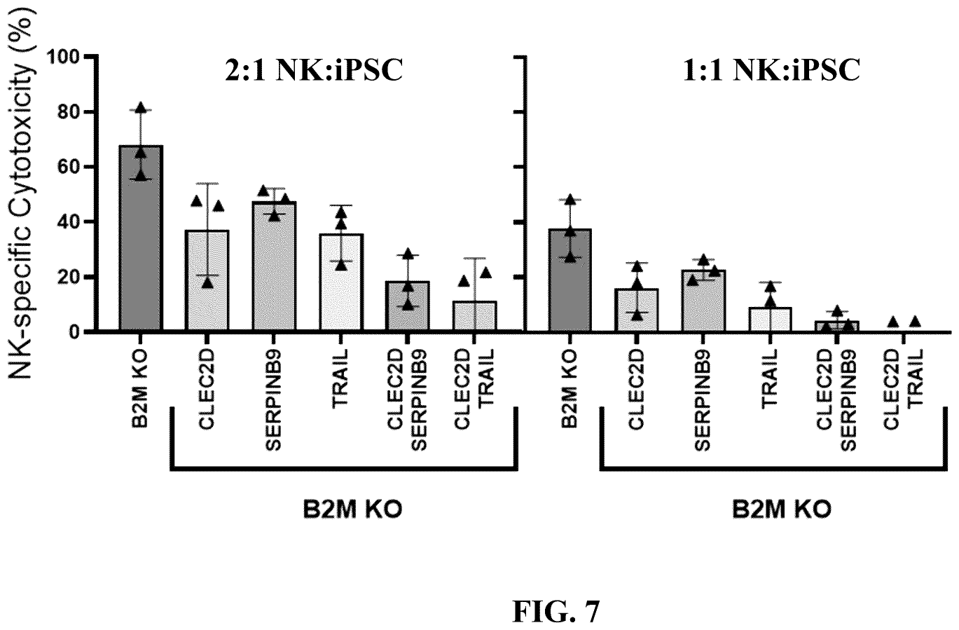

FIGS. 6 and 7 show exemplary results from NK cell cytotoxicity assays. These results demonstrate that individual expression of CLEC2D, SERPINB9, or TRAIL in wild-type iPSCs and B2M KO iPSCs reduced NK cell-mediated cytotoxicity to varying degrees. Combining CLEC2D with either SERPINB9 or TRAIL in iPSCs engineered with a B2M KO further reduced NK cell-mediated cytotoxicity as compared to individual gene expression. Despite a higher level of NK cell-mediated cytotoxicity observed at a higher NK cell to iPSCs ratio, a reduction in NK cell-mediated cytotoxicity was observed with individual expression of CLEC2D, SERPINB9, or TRAIL (FIG. 7). These results demonstrate the effectiveness of combining specific gene targets in protecting iPSCs against NK cell-mediated cytotoxicity

### Example 6: Evaluation of NK Cell Cytotoxicity Against Cardiomyocytes Engineered with Multiple Immune Evasion Genes

This example describes experiments that were conducted to evaluate the ability of cardiomyocytes engineered to express multiple immune evasion genes to evade NK cell-mediated cytotoxicity in vitro.

iPSCs with a B2M knockout were engineered to express CLEC2D individually, as well as in combination with SERPINB9 (CLEC2D+SERPINB9). The engineered iPSCs were then differentiated into cardiomyocytes. Control cardiomyocytes with only a B2M knockout were also included.

The engineered cardiomyocytes were subjected to in vitro NK cell cytotoxicity assays as described in Example 2. These cells were co-cultured with NK cells at two different NK cell to cardiomyocyte ratios (2:1 and 1:1), and the percentage of NK-specific cytotoxicity was measured.

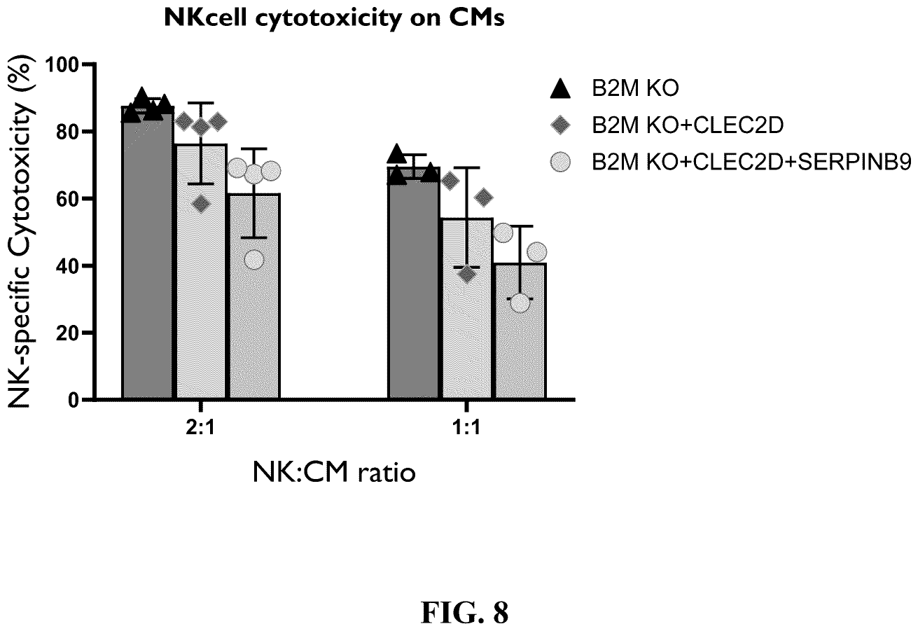

FIG. 8 shows exemplary results from NK cell cytotoxicity assays. These results demonstrate that expression of CLEC2D in B2M KO cardiomyocytes reduced NK cell-mediated cytotoxicity compared to B2M KO cardiomyocytes alone. Combining CLEC2D with SERPINB9 in cardiomyocytes engineered with a B2M KO further reduced NK cell-mediated cytotoxicity as compared to individual CLEC2D expression. This protective effect was observed at both NK cell to cardiomyocyte ratios tested (2:1 and 1:1), though higher levels of NK cell-mediated cytotoxicity were observed at the higher NK cell to cardiomyocyte ratio. These results demonstrate the effectiveness of combining specific gene targets in protecting cardiomyocytes against NK cell-mediated cytotoxicity, including in the context of B2M knockout cells.

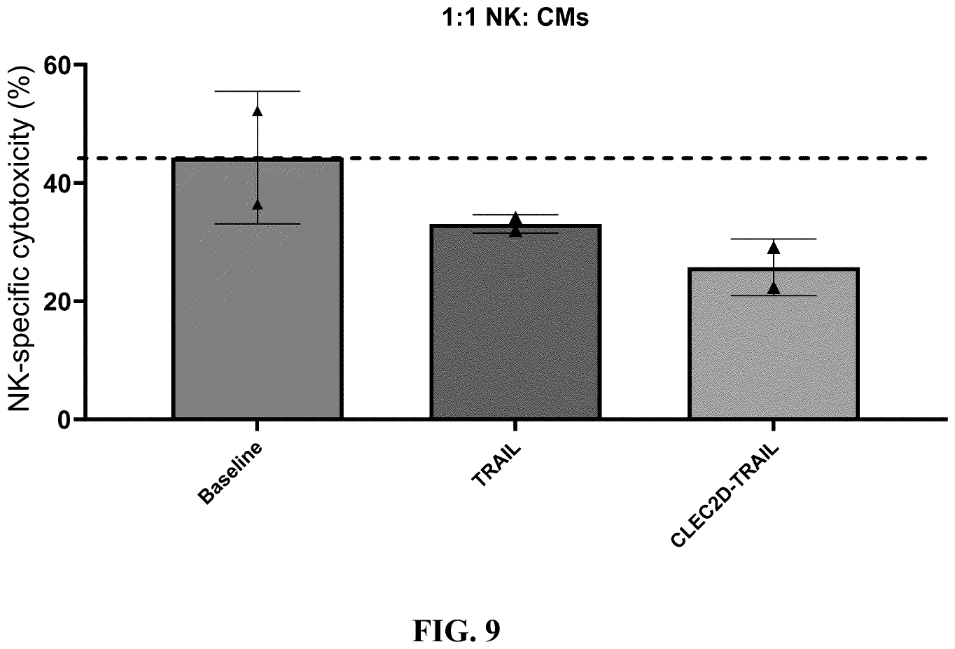

Further multiplex experiments were conducted using methods similar to those described herein to evaluate potential synergistic effects between TRAIL and CLEC2D in protecting cardiomyocytes against NK cell-mediated cytotoxicity. FIG. 9 shows exemplary results from NK cell cytotoxicity assays conducted at a 1:1 NK cell to cardiomyocyte ratio. Cardiomyocytes expressing TRAIL alone showed moderate protection against NK cell-mediated killing compared to baseline control cells. However, cardiomyocytes expressing both CLEC2D and TRAIL demonstrated enhanced protection, with NK-specific cytotoxicity reduced to approximately 25% compared to baseline levels of about 45%. These results further support the effectiveness of combining immune evasion gene targets, including CLEC2D and TRAIL, to achieve greater protection against NK cell-mediated cytotoxicity in cardiomyocytes.

### EQUIVALENTS AND SCOPE, INCORPORATION BY REFERENCE

Those skilled in the art will recognize, or be able to ascertain using no more than routine experimentation, many equivalents to the specific embodiments described herein. It is understood that modifications which do not substantially affect the activity of the various embodiments of this disclosure are also provided within the description of the disclosure provided herein. The scope of the present disclosure is not intended to be limited to the above description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure also includes embodiments in which more than one, or all of the group members, are present in, employed in, or otherwise relevant to a given product or process.

Furthermore, it is to be understood that the disclosure encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should be understood that, in general, where the disclosure, or aspects of the embodiments, is/are referred to as that includes particular elements, features, steps, etc., certain embodiments of the disclosure or aspects of the embodiments consist, or consist essentially of, such elements, features, steps, etc. Thus, for each embodiment of the disclosure that includes one or more elements, features, steps, etc., the disclosure also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the disclosure, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

Throughout this disclosure various publications, patents, and sequence database entries are mentioned. The disclosures of these publications, patents, and sequence database entries, including those items listed above, are hereby incorporated by reference in their entirety as if each individual publication or patent was specifically and individually indicated to be incorporated by reference. In case of conflict, the present application, including any definitions herein, will control.

Although the disclosure has been described with reference to the examples provided above, it should be understood that various modifications can be made without departing from the scope of the disclosure. Accordingly, the above examples are intended to illustrate but not limit the present disclosure.

What is claimed is:

1. A genetically modified stem cell comprising a heterologous nucleic acid sequence encoding C-type lectin domain family 2 member D (CLEC2D), wherein CLEC2D comprises an amino acid sequence at least 95% identical to the sequence set forth in SEQ ID NO: 2.

2. The genetically modified stem cell of claim 1, further comprising one or two heterologous nucleic acid sequences selected from the group consisting of:
(i) a heterologous nucleic acid sequence encoding serpin family B member 9 (SERPINB9); and
(ii) a heterologous nucleic acid sequence encoding Tumor Necrosis Factor-related apoptosis-inducing ligand (TRAIL).

3. The genetically modified stem cell of claim 2, comprising the heterologous nucleic acid sequence encoding SERPINB9, wherein SERPINB9 comprises an amino acid sequence at least 95% identical to the sequence set forth in SEQ ID NO: 6.

4. The genetically modified stem cell of claim 2, comprising the heterologous nucleic acid sequence encoding TRAIL, wherein TRAIL comprises an amino acid sequence at least 95% identical to the sequence set forth in SEQ ID NO: 4.

5. The genetically modified stem cell of claim 2, comprising the heterologous nucleic acid sequence encoding SERPINB9 and the heterologous nucleic acid sequence encoding TRAIL, wherein SERPINB9 comprises an amino acid sequence at least 95% identical to the sequence set forth in SEQ ID NO: 6 and TRAIL comprises an amino acid sequence at least 95% identical to the sequence set forth in SEQ ID NO: 4.

6. The genetically modified stem cell of claim 1, wherein expression of the heterologous nucleic acid sequence encoding CLEC2D is driven by a promoter of an endogenous gene.

7. The genetically modified stem cell of claim 6, wherein the endogenous gene comprises a glyceraldehyde 3-phosphate dehydrogenase (GAPDH) gene.

8. The genetically modified stem cell of claim 1, wherein the genetically modified stem cell is least 20% less susceptible to natural killer cell mediated cytotoxicity as compared to a wild-type stem cell.

9. The genetically modified stem cell of claim 1, wherein the modified stem cell comprises a human pluripotent stem cell.

10. The genetically modified stem cell of claim 1, wherein the genetically modified stem cell further comprises a genetic modification that results in reduced T cell-mediated killing as compared to a wild-type stem cell.

11. The genetically modified stem cell of claim 10, wherein the genetic modification comprises:
(i) a deletion, disruption, or attenuation of a β₂ microglobulin (B2M) gene; or
(ii) a deletion, disruption, or attenuation of one or more of an HLA-A gene, an HLA-B gene, and an HLA-C gene.

12. The genetically modified stem cell of claim 11, wherein the genetically modified stem cell further comprises a deletion, disruption, or attenuation of a class II, major histocompatibility complex, transactivator (CIITA) gene or a regulatory factor X5 (RFX5) gene.

13. The genetically modified stem cell of claim 1, comprising:
(i) the heterologous nucleic acid sequence encoding C-type lectin domain family 2 member D (CLEC2D), wherein CLEC2D comprises an amino acid sequence at least 95% identical to the sequence set forth in SEQ ID NO: 2;
(ii) a second heterologous nucleic acid sequence encoding serpin family B member 9 (SERPINB9), wherein SERPINB9 comprises an amino acid sequence at least 95% identical to the sequence set forth in SEQ ID NO: 6;
(iii) a third heterologous nucleic acid sequence encoding Tumor Necrosis Factor-related apoptosis-inducing ligand (TRAIL), wherein TRAIL comprises an amino acid sequence at least 95% identical to the sequence set forth in SEQ ID NO: 4; and
(iv) a genetic modification that results in reduced T cell-mediated killing as compared to a wild-type stem cell.

14. A genetically modified cell comprising a heterologous nucleic acid sequence encoding C-type lectin domain family 2 member D (CLEC2D), wherein CLEC2D comprises an amino acid sequence at least 95% identical to the sequence set forth in SEQ ID NO: 2, and wherein the genetically modified cell is derived from a genetically modified stem cell.

15. The genetically modified cell of claim 14, further comprising one or two heterologous nucleic acid sequences selected from the group consisting of:
(i) a heterologous nucleic acid sequence encoding serpin family B member 9 (SERPINB9); and
(ii) a heterologous nucleic acid sequence encoding Tumor Necrosis Factor-related apoptosis-inducing ligand (TRAIL).

16. The genetically modified cell of claim 15, comprising the heterologous nucleic acid sequence encoding SERPINB9, wherein SERPINB9 comprises an amino acid sequence at least 95% identical to the sequence set forth in SEQ ID NO: 6.

17. The genetically modified cell of claim 15, comprising the heterologous nucleic acid sequence encoding TRAIL, wherein TRAIL comprises an amino acid sequence at least 95% identical to the sequence set forth in SEQ ID NO: 4.

18. The genetically modified cell of claim 15, comprising the heterologous nucleic acid sequence encoding SERPINB9 and the heterologous nucleic acid sequence encoding TRAIL, wherein SERPINB9 comprises an amino acid sequence at least 95% identical to the sequence set forth in SEQ ID NO: 6 and TRAIL comprises an amino acid sequence at least 95% identical to the sequence set forth in SEQ ID NO: 4.

19. The genetically modified cell of claim 14, wherein expression of the heterologous nucleic acid sequence encoding CLEC2D is driven by a promoter of an endogenous gene.

20. The genetically modified cell of claim 19, wherein the endogenous gene comprises a glyceraldehyde 3-phosphate dehydrogenase (GAPDH) gene.

21. The genetically modified cell of claim 14, wherein the genetically modified cell further comprises a genetic modification that results in reduced T cell-mediated killing as compared to a wild-type cell.

22. The genetically modified stem cell of claim 21, wherein the genetic modification comprises:
(i) a deletion, disruption, or attenuation of a β₂ microglobulin (B2M) gene; or
(ii) a deletion, disruption, or attenuation of one or more of an HLA-A gene, an HLA-B gene, and an HLA-C gene.

23. The genetically modified cell of claim 22, wherein the genetically modified cell further comprises a deletion, disruption, or attenuation of a class II, major histocompatibility complex, transactivator (CIITA) gene or a regulatory factor X5 (RFX5) gene.

24. The genetically modified cell of claim 14, wherein the genetically modified cell comprises a cardiac cell, a neural cell, a T cell, a retinal cell, an endocrine cell, an epithelial cell, a muscle cell, or a myeloid cell.

25. The genetically modified cell of claim 24, wherein the genetically modified cell comprises a cardiac cell.

26. A method of genetically modifying a stem cell, comprising introducing into the stem cell one or more heterologous nucleic acid sequences selected from the group consisting of:
(i) a heterologous nucleic acid sequence encoding C-type lectin domain family 2 member D (CLEC2D), wherein CLEC2D comprises an amino acid sequence at least 95% identical to the sequence set forth in SEQ ID NO: 2,
(ii) a heterologous nucleic acid sequence encoding serpin family B member 9 (SERPINB9), wherein SERPINB9 comprises an amino acid sequence at least 95% identical to the sequence set forth in SEQ ID NO: 6, and
(iii) a heterologous nucleic acid sequence encoding Tumor Necrosis Factor-related apoptosis-inducing ligand (TRAIL), wherein TRAIL comprises an amino acid sequence at least 95% identical to the sequence set forth in SEQ ID NO: 4;
wherein the genetically modified stem cell expresses the one or more heterologous nucleic acid sequences, and
wherein expression of the one or more heterologous nucleic acid sequences is maintained following differentiation of the genetically modified stem cell.

27. A method of making the genetically modified stem cell of claim 1, comprising introducing into a stem cell the heterologous nucleic acid sequence encoding C-type lectin domain family 2 member D (CLEC2D).

28. A method of making the genetically modified cell of claim 14, comprising:
(I) introducing into a stem cell the heterologous nucleic acid sequence encoding C-type lectin domain family 2 member D (CLEC2D), to produce a genetically modified stem cell, and
(II) differentiating the genetically modified stem cell of step (I) to produce the genetically modified cell.

29. The method of claim 28, wherein the genetically modified cell produced in step (II) comprises a cardiac cell, a neural cell, a T cell, a retinal cell, an endocrine cell, an epithelial cell, a muscle cell, or a myeloid cell.
